SEQUENCE LISTING (132 sequences)

SEQ ID NO: 1 - protein, 328 aa, Solanum lycopersicum
MAVNGASSSGLIVSFGEMLIDFVPTVSGVSLAEAPGFLKAPGGAPANVAIAVTRLGGKSAFVGKLGDDEFGHMLAGILKTNGVQAEGINFDKGARTALAFVTLRADGEREFMFYRNPSADMLLTPAELNLDLIRSAKVFHYGSISLIVEPCRAAHMKAMEVAKEAGALLSYDPNLRLPLWPSAEEAKKQIKSIWDSADVIKVSDVELEFLTGSNKIDDESAMSLWHPNLKLLLVTLGEKGCNYYTKKFHGTVGGFHVKTVDTTGAGDSFVGALLTKIVDDQTILEDEARLKEVLRFSCACGAITTTKKGAIPALPTASEALTLLKGGA

SEQ ID NO: 2 - protein, 325 aa, Camelina sativa
MASNGEKGLIVSFGEMLIDFVPTESGVSLAEAPGFLKAPGGAPANVAIAVSRLGGRSAFVGKLGDDEFGHMLAGILRKNGVADQGINFDTGARTALAFVTLRADGDREFMFYRNPSADMLLRPDELNLDLIRSAKVFHYGSISLIVEPCRSAHLKAMEVAKEAGALLSYDPNLREPLWPSKEEAKTQIMSIWDKAEIIKVSDVELEFLTGSNKIDDETAMSLWHPNLKLLLVTLGEKGCRYYTKSFKGSVDPFHVNAVDTTGAGDSFVGALLNQIVDDQSVLENEERLRKVLRFANACGAITTTKKGAIPALPSDAEVRSFLEGK

SEQ ID NO: 3 - protein, 325 aa, Brassica napus
MASNGDKPLIVSFGEMLIDFVPTESGVSLAEAPGFLKAPGGAPANVAIAVSRLGGRSAFVGKLGDDEFGHMLAGILRKNGVADQGINFDTGARTALAFVTLKADGDREFMFYRNPSADMLLRPDELNLELIRSAKVFHYGSISLIVEPCRSAHLKAMEVAKEAGALLSYDPNLREPLWPSKEEAKTQIMSIWDKAEIIKVSDVELEFLTGSNKIDDETAMSLWHPNLKLLLVTLGEKGCRYYAKNFRGSVDPFHVNAVDTTGAGDSFVGALLNKIADDHSILEDEERLRKVLRFANACGAITTTKKGAIPALPSDAEVLSFLEGK

SEQ ID NO: 4 - protein, 328 aa, Populus euphratica
MASNGLNDKSLIVSFGEMLIDFVPTTSGVSLAEAPGFLKAPGGAPANVAIAVARLGGKSAFVGKLGDDEFGHMLAGILKENNVIASGINFDTGARTALAFVTLRADGEREFMFYRNPSADMLLRPEELNLELIRSAKVFHYGSISLIVEPCRSAHLEAMKVAKEAGALLSYDPNLRLPLWPSAEEAREQILKIWDEADVIKVSDVELEFLTGSNKIDDETALTLWRPNFKLLLVTLGENGCKYYTKTFHGTVEAFHVKTIDTTGAGDAFIGALLCKIVDDQSVLEDEQKLREILRFANACGAITTTKKGAIPALPTPAEVHKLLNASK

SEQ ID NO: 5 - protein, 328 aa, Capsicum annuum
MAVNGSASSGLIVSFGEMLIDFVPTVSGVSLAEAPGFLKAPGGAPANVAIAVTRLGGKSAFVGKLGDDEFGHMLAGILKQNGVQADGINFDTGARTALAFVTLRADGEREFMFYRNPSADMLLKPEELNLDLIRSAKVFHYGSISLIVEPCRSAHLKAMEVAKEAGALLSYDPNLRLPLWPSAEEAKKQIKSIWDKADVIKVSDVELEFLTGNPKIDDESAMSLWHPNLKLLLVTLGEKGCNYYTKNFRGSVGAFHVKTIDTTGAGDSFVGALLTKIVDDQAIIQDEARLKEVLRFACACGAITTTKKGAIPALPTEADALTLIKGGA

SEQ ID NO: 6 - protein, 325 aa, Noccaea caerulescens
MASNGEKGLIVSFGEMLIDFVPTESGVSLAEAPGFLKAPGGAPANVAIAVSRLGGRSAFVGKLGDDEFGHMLAGILRKNGVADQGINFDTGARTALAFVTLRADGDREFMFYRNPSADMLLRPDELNLELIRSAKVFHYGSISLIVEPCRSAHLKAMEVAKEAGALLSYDPNLREPLWPSKEEAKTQIMSIWDKAEIIKVSDVELEFLTGSNKIDDETAMSLWHPNLKLLLVTLGENGCRYYAKTFRGSVDPFHVNAVDTTGAGDSFVGALLNKIADDQSILEDEERLRKVLRFANACGAITSTKKGAIPALPSEAEVVSFLQGK

SEQ ID NO: 7 - protein, 325 aa, Brassica rapa
MASNGEKPLIVSFGEMLIDFVPTESGVSLAEAPGFLKAPGGAPANVAIAVSRLGGRSAFVGKLGDDEFGHMLAGILRKNGVADQGINFDTGARTALAFVTLKADGDREFMFYRNPSADMLLRPDELNLELIRSAKVFHYGSISLIVEPCRSAHLKAMEVAKEAGALLSYDPNLREPLWPSKEEAKTQIMSIWDKAEIIKVSDVELEFLTGSNKIDDETAMSLWHPNLKLLLVTLGEKGCRYYAKNFRGSVDPFHVNAVDTTGAGDSFVGALLNKIADDHSILEDEERLRKVLRFANACGAITTTKKGAIPALPSDAEVLSFLEGK

SEQ ID NO: 8 - protein, 325 aa, Brassica oleracea
MASNGDKPLIVSFGEMLIDFVPTESGVSLAEAPGFLKAPGGAPANVAIAVSRLGGRSAFVGKLGDDEFGHMLAGILRKNGVADQGINFDTGARTALAFVTLKADGDREFMFYRNPSADMLLRPDELNLQLIRSAKVFHYGSISLIVEPCRSAHLKAMEVAKEAGALLSYDPNLREPLWPSKEEAKTQIMSIWDKAEIIKVSDVELEFLTGSNKMDDETAMSLWHPNLKLLLVTLGEKGCRYYAKNFRGSVDPFHVNAVDTTGAGDSFVGALLNKIADDHSILEDEERLRKVLRFANACGAITTTKKGAIPALPSDAEVLSFLEGK

SEQ ID NO: 9 - protein, 325 aa, Raphanus sativus
MASNGEKPLIVSFGEMLIDFVPTESGVSLAEAPGFLKAPGGAPANVAIAVSRLGGRSAFVGKLGDDEFGHMLAGILRKNGVADQGINFDTGARTALAFVTLRADGDREFMFYRNPSADMLLRPDELNLELIRSAKVFHYGSISLIVEPCRSAHLKAMEVAKEAGALLSYDPNLREPLWPSKEEAKTQIMSIWDKAEIIKVSDVELEFLTGSNKIDDETAMSLWHPNLKLLLVTLGEKGCRYYAKNFRGSVDPFHVNAVDTTGAGDSFVGALLNKIADDQSILEDEERLRKVLRFANACGAITTTKKGAIPALPSDAEVLSFLEGK

SEQ ID NO: 10 - protein, 331 aa, Eucalyptus grandis
MASRNNGISGGKGLIVSFGEMLIDFVPTVSGVSLAEAPGFLKAPGGAPANVAIAVTRLGGRSAFVGKLGDDEFGHMLAGILKENGVNCDGINFDQGARTALAFVTLRADGEREFMFYRNPSADMLLKPEELNLELIRSAKVFHYGSISLIVEPCRSAHLEAMQVAKDAGALLSYDPNLRLPLWPSPEEAREQIKSIWDKADIIKVSDVELEFLTGSDKIDDENAMTLWHPNLTLLLVTLGEHGCRYYTKHFHGHVEAFHVNTVDTTGAGDSFVGALLCNIVDDRSILEDEGKLRKVLKFANACGAITTTKKGAIPALPTEADVLSLIDGTK

SEQ ID NO: 11 - protein, 328 aa, Camelina sativa
MTSSTVDKGLIVSFGEMLIDFVPTESGVSLSESQGFLKAPGGAPANVAIAVSRLGGRAAFVGKLGDDEFGHMLAEILRKNGVADQGINFDKGARTALAFVTLRSDGEREFMFYRNPSADMLLRPDELNLELIRSAKVFHYGSISLITEPCRSAHMKAMEVAKEAGALLSYDPNLREPLWPSPEEARTQIMSIWDKADVIKVSDVELEFLTGNKTIDDETAMSLWHPNLKLLLVTLGEKGCRYYTKGFHGSVETFHVDAVDTTGAGDSFVGALLNQIVDDQSVFEEEERLRQVLRFANACGAITTTKKGAIPALPSDCEALSFLKGQVD

SEQ ID NO: 12 - protein, 367 aa, Brassica napus
MTSKALNHVLHPYINYTNLRSLILISIVFLLRSLEHKLELVPMASNLDKGLIVSFGEMLIDFVPTQSGVSLAESTGFLKAPGGAPANVAIAVTRLGGRAAFVGKLGDDEFGHMLAGILRENGVEDKGINFDKGARTALAFVTLRSDGDREFMFYRNPSADMLLRPDELNLELIRSAKVFHYGSISLITEPCRSAHLKAMEVAKEAGALLSYDPNLREPLWSSPEEARKQIMSIWDKAEIIKVSDVELEFLTQNKTIDDESAMSLWHPNLKLLLVTLGEKGCRYYTKGFHGSVEAFDVNAVDTTGAGDSFIGAFLSQIVDDQTVLKEEERLRKVLRFANACGAITTTKKGAIPALPSECDALSLLKDK

SEQ ID NO: 13 - protein, 331 aa, Glycine max
MASSTNALPPTGNGLIVSFGEMLIDFVPTVSGVSLAEAPGFLKAPGGAPANVAIAVARLGGKAAFVGKLGDDEFGHMLAGILKENDVRSDGINFDQGARTALAFVTLRADGEREFMFYRNPSADMLLTPEDLNLELIRSAKVFHYGSISLIVEPCRSAHLKAMEVAREAGCLLSYDPNLRLPLWPSAEEARQQILSIWDKADVIKVSDVELEFLTGSDKIDDASALSLWHPNLKLLLVTLGEHGSRYYTKNFHGSVEAFHVSTVDTTGAGDSFVGALLSKIVDDQSVLEDEARLREVLKFANACGAITTTKKGAIPALPTEADALNLIKEA

SEQ ID NO: 14 - protein, 329 aa, Capsella rubella
MASSTGEKGLIVSFGEMLIDFVPTVSGVSLSQSPGFVKAPGGAPANVAIAVSRLGGRAAFVGKLGDDEFGHMLAGILRKNGVADQGISFDKGARTALAFVTLRSDGEREFMFYRNPSADMLLRPDELNLELIRSAKVFHYGSISLITEPCRSAHMKAMEVAKEAGALLSYDPNLREPLWPSPEEARTQILSIWDKADIIKVSDVELEFLTGNKTIDDETAMSLWHPNLKLLLVTLGENGCRYYTENFHGSVETFHVDAVDTTGAGDSFVGALLNQIVDDQSVFEEEERLKKVLRFANACGAITTTKKGAIPALPSDCEALSFLERQAEM

SEQ ID NO: 15 - protein, 368 aa, Noccaea caerulescens
SKASNSVFTLLNPYINHSTLRSLFILSIIFIHRSFERKIDLAQMASNGEKGLIVSFGEMLIDFVPTESGVSLAESPGFVKAPGGAPANVAIAVSRLGGRAAFVGKLGDDEFGHMLAGILRENGVEDKGINFDKGARTALAFVTLRSDGDREFMFYRNPSADMLLRPDELNLELIRSAKVFHYGSISLITEPCRSAHLKAMEVAKEAGALLSYDPNLREPLWPSPEEAKTQIMSIWDKAEIIKVSDVELEFLTGNKTIDDESAMSLWHPNLKLLLVTLGEKGCRYYTQNFHGSVEAFDVNAVDTTGAGDSFIGAFLSQIVDDHSVLEEEERLRKALRFANAVGAITTTKKGAVPALPTDCEAHCFLKDK

SEQ ID NO: 16 - protein, 367 aa, Brassica rapa
MTSKALNHVLHPYINYTNLRSLILISIVFLLRSLEHKLELVPMASNLDKGLIVSFGEMLIDFVPTQSGVSLAESPGFLKAPGGAPANVAIAVTRLGGRAAFVGKLGDDEFGHMLAGILRENGVEDKGINFDKGARTALAFVTLRSDGDREFMFYRNPSADMLLRPDELNLELIRSAKVFHYGSISLITEPCRSAHLKAMEVAKEAGALLSYDPNLREPLWSSPEEARKQIMSIWDKAEIIKVSDVELEFLTQNKTIDDESAMSLWHPNLKLLLVTLGEKGCRYYTKGFHGSVEAFDVNAVDTTGAGDSFIGAFLSQIVDDQTVLKEEERLRKVLRFANACGAITTTKKGAIPALPSECDALSLLKDK

SEQ ID NO: 17 - protein, 367 aa, Brassica oleracea
MTSKPLNHVLHPYINYTNLRSLILISIVFLLRSLEHKLELVPMASNLDKGLIVSFGEMLIDFVPTQSGVSLAESTGFLKAPGGAPANVAIAVTRLGGRAAFVGKLGDDEFGHMLAGILRENGVEDKGINFDKGARTALAFVTLRSDGDREFMFYRNPSADMLLRPDELNLELIRSAKVFHYGSISLITEPCRSAHLKAMEVAKEAGALLSYDPNLREPLWSSPEEARTQIMSIWDKAEIIKVSDVELEFLTQNKTIDDESAMSLWHPNLKLLLVTLGEKGCRYYTKGFHGSVEAFDVNAVDTTGAGDSFIGAFLSQIVDDQTVLEEEERLRKVLRFANACGAITTTKKGAIPALPSECDALSLLKDK

SEQ ID NO: 18 - protein, 371 aa, Raphanus sativus
MTSKALNHLFTLLNHYINYTNLRSLIILSIIFVLRSLEHKLDLVPMSSNVEKKGLIVSFGEMLIDFVPTESGVSLAQSPGFLKAPGGAPANVAIAVTRLGGRAAFVGKLGDDEFGHMLAGILRENGVEDKGINFDKGARTALAFVTLRSDGDREFMFYRNPSADMLLRPDELNLELIRSAKVFHYGSISLITEPCRSAHLKAMEVAKEAGALLSYDPNLREPLWSSPEEARTQIMSIWDKAEIIKVSDVEIEFLTQNKTIDDESAMSLWHPNLKLLLVTLGEKGCRYYTKGFHGSVEAFDVSAVDTTGAGDSFIGAFLSQIVDDQTVLEEEERLRKVLRFANACGAITTTKKGAIPALPSECEALSLLKDK

SEQ ID NO: 19 - protein, 327 aa, Nicotiana tabacum
MAANGVSSGLIVSFGEMLIDFVPTVSGVSLAEAPGFLKAPGGAPANVAIAVTRLGGKSAFVGKLGDDEFGHMLAGILKQNGVQADGINFDKGARTALAFVTLRADGEREFMFYRNPSADMLLTPDELNLEVIRSAKVFHYGSISLIVEPCRSAHLKAMEVAKEAGALLSYDPNLRLPLWPSAEEARKQIKSIWDKADVIKVSDVELEFLTGSDKIDDESAMSLWHPNLKLLLVTLGEKGCNYYTKNFHGGVEAFHVKTVDTTGAGDSFVGALLTKIVDDQSILEDEARLKEVLRFACACGAITTTKKGATPALPTESEALTLLNGGA

SEQ ID NO: 20 - protein, 330 aa, Ricinus communis
MASVTNGKSSSSLIVSFGEMLIDFIPTVSGVSLAEAPGFLKAPGGAPANMAIAVARLGGNAAFVGKLGDDKFGHMLAGILKENGVGSDGINFDKGARTALAFVTLRADGEREFMFYRNPSADMLLTPEELNLEVIRSAKIFHYGSISLIVEPCRSAHLKAMEEAKNAGALLSYDPNLRLPLWPSAEYAREQIMSIWDKADIIKVSDVELEFLTGSDKIDDESALSLWHPNLKLLLVTLGENGCRYYTKNFHGSVDAFHVKTVDTTGAGDSFVGALLCKIVDDLSVLEEEPRLREVLRFANACGAITTTKKGAIPALPTEADVLSLMKASS

SEQ ID NO: 21 - protein, 330 aa, Cicer arietinum
MASNTPIPSTGEGLIVSFGEMLIDFVPTVSGVSLAEAPGFLKAPGGAPANVAIAVSRLGGKAAFVGKLGDDEFGHMLAGILKENNVRADGINFDKGARTALAFVTLRADGEREFMFYRNPSADMLLTPEDLNLELIRSAKVFHYGSISLIVEPCRSAHLKAMEVAKDAGCLLSYDPNLRLPLWPSPEEARNQILSIWDKADLIKVSDVELEFLTGSDKIDDASALSLWHPNLKLLLVTLGENGSRYYTKNFHGSVDAFHVNTVDTTGAGDSFVGALLGKIVDDQSILEDEARLREVLKFANACGAITTTKKGAIPALPTEADVLSLIKGE

SEQ ID NO: 22 - protein, 329 aa, Citrus clementina
MATNGAVSSSSSLIVSFGEMLIDFVPTVSGVSLAEAPGFLKAPGGAPANVAIAVARLGGKAAFVGKLGDDEFGHMLAGILKENGVSADGINFDQGARTALAFVTLRADGEREFMFYRNPSADMLLRPDELNLELIRSAKVFHYGSISLIVEPCRSAHLEAMKAAKEAGALLSYDPNLRLPLWPSPEEAREQIMSIWDKAEVIKVSDVELEFLTGSDKIDDESALSLWHPNLKLLLVTLGEHGCRYYTKSFRGAVDAFHVNTVDTTGAGDAFVGALLGKLVDDQSVLEDEPRLREILKFANACGAITTTKKGAIPALPTESEALNLLKGA

SEQ ID NO: 23 - protein, 347 aa, Vigna radiata
MASANALPPTGNGLVVSFGEMLIDFVPTVSGVSLAEAPGFLKAPGGAPANVAIAVARLGGKAAFVGKLGDDEFGHMLAGILKENDVRSDGINFDQGARTALAFVTLRKDGEREFMFYRNPSADMLLTPEDLNLELIRSAKVFHYGSISLIVEPCRSAHLKAMEVAREAGCLLSYDPNLRLPLWPSPEEARQQILSIWEKADVIKVSDVELEFLTGSNKIDDESALSLWHPNLKLLLVTLGEHGSRYYTKNFHGSVDAFHVNTVDTTGAGDSFVGALLCKIVDDQSILEDEAKLREVLKFANACGAITTTKKGAIPALPTEADALNLINEKNGKEGVLEALKSRCIIL

SEQ ID NO: 24 - protein, 333 aa, Juglans regia
MASWLNNNGVPESGSGLIVSFGEMLIDFVPTVSGVSLAEAPGFLKAPGGAPANVAIAVSRLGGKAAFVGKLGDDEFGHMLAGILDQNGVAGNGINFDQGARTALAFVTLRADGEREFMFYRNPSADMLLRPEELNLELIRSAKVFHYGSISLIVEPCRSAHLKAMEVAKDAGALLSYDPNLRLPLWPSAEEACKQIKSIWDKADIIKVSDVELDFLTGSDKCDDAAALSLWHPNLKLLLVTLGEKGCRYYTKNFHGEVEAFHVKTVDSTGAGDSFVGALLSKIVDDQSILEDEPRLRQVLKFANACGAITTTKKGAIPALPTESEVCSLLKGA

SEQ ID NO: 25 - protein, 347 aa, Camelina sativa
MASSTGEKGLIVSFGEMLIDFVPTVSGVSLSESPGFLKAPGGAPANVAIAVSRLGGRSAFVGKLGDDDFGHMLAGILRKNGVADEGVNFDKGARTALAFVTLRSDGEREFMFYRNPSADMLLRPDELNLELIRSAKVFHYGSISLITEPCRSAHMKAMEVAKEAGALLSYDPNLREPLWPSPEEARTQIMSIWDKADIIKVSDVELEFLTENKTMDDKTAMSLWHPNLKLLLVTLGEKGCTYFTKKFHGTVETFPVDAVDTTGAGDSFVGALLHQIVDDQSVLEDEVRLRKVLRFANACGAITTTKKGAIPALPSDSEAHSFLKDKKKRQTYLKFSKLCCTASPCQL

SEQ ID NO: 26 - protein, 345 aa, Brassica napus
MSSTREKGLIVSFGEMLIDFVPTESGVSLAESPGFLKAPGGAPANVAIAVTRLGGRSAFVGKLGGDEFGHMLAGILRENGVDDTGVSFDKGARTALAFVTLRSDGEREFMFYRNPSADMLLRPDELNLELIRSAKVFHYGSISLITEPCRSAHLKAMEVAKEAGALLSYDPNLREPLWPSPEEARKQIMSVWDKAEIIKVSDVELEFLTENKTIDDESAMSLWHPNLKLLLVTLGEDGCRYYTKKFHGAVQTFNVNAVDTTGAGDSFVGAFLSKIVDDQSVLEEEKKLRKVLRFANACGAITTTKKGAIPALPSDSEALSFIKDNQRRKENSFRVGYVSCLHSSL

SEQ ID NO: 27 - protein, 326 aa, Camelina sativa
MAASNGEKSLIVSFGEMLIDFVPTVSGVSLAEAPGFIKAPGGAPANVAIAISRLGGRAAFVGKLGDDEFGHMLAGILKQNGVAAEGINFDTGARTALAFVTLRSDGEREFMFYRNPSADMLLRPDELNLDLIRSAKVFHYGSISLIVEPCRSAHLKAMEVAKEAGALLSYDPNLRLPLWPSKEEAQKQILSIWDKAEVIKVSDEELMFLTGSDKVDDETALSLWHSNLKLLLVTLGDKGCRYYTKSFRGSVDPFHVNAVDTTGAGDAYVGALLCKIVDDRAVLEDEPRLREVLRFANACGAITTTKKGAIPALPTEAEVQSLLKGN

SEQ ID NO: 28 - protein, 326 aa, Brassica napus
MTTSNGEKSLIVSFGEMLIDFVPTVSGVSLAEAPGFIKAPGGAPANVAIAVSRLGGRAAFVGKLGDDEFGHMLAGILKQNGVSAEGINFDTGARTALAFVTLRSDGEREFMFYRNPSADMLLRPDELNLDLIKSAKVFHYGSISLIVEPCRSAHLKAMEVAKESGALLSYDPNLRLPLWPSKEEAKKQILSIWDKAEVIKVSEEELMFLTGSDNVDDETALSLWHSNLKLLLVTLGEKGCNYYTKSFRGSVDPFHVNAVDTTGAGDSYVGALLCNIVDDRSVLEDEARLREVLRFANACGAITTTKKGAIPALPTESEVQSLLNGN

SEQ ID NO: 29 - protein, 326 aa, Brassica napus
MAASNGEKSLIVSFGEMLIDFVPTVSGVSLAEAPGFIKAPGGAPANVAIAVSRLGGRAAFVGKLGDDEFGHMLAGILKQNGVSAEGINFDTGARTALAFVTLRSDGEREFMFYRNPSADMLLRPDELNLDLIKSAKVFHYGSISLIVEPCRSAHLKAMEVAKEAGALLSYDPNLRLPLWPSKEEAKKQILSIWDKAELIKVSDEELMFLTGSDNVDDETALSLWHDNLKLLLVTLGEKGCKYYTKSFRGSVDPFHVNTVDTTGAGDSFVGALLCNIVDDRSVLEDEARLREVLRFANACGAITTTKKGAIPALPTVSEVQTLLNGN

SEQ ID NO: 30 - protein, 331 aa, Glycine max
MALNNGVPATGTGLIVSFGEMLIDFVPTVSGVSLAEAPGFLKAPGGAPANVAIAVSRLGGKAAFVGKLGDDEFGHMLAGILKENGVRADGINFDQGARTALAFVTLRADGEREFMFYRNPSADMLLKPEELNLELIRSAKVFHYGSISLIVEPCRSAHLKAMEVAKESGCLLSYDPNLRLPLWPSAEEARKQILSIWEKADLIKVSDAELEFLTGSDKIDDESALSLWHPNLKLLLVTLGEHGSRYYTKSFKGSVDAFHVNTVDTTGAGDSFVGALLAKIVDDQSILEDEPRLREVLKFANACGAITTTQKGAIPALPKEEAALKLIKGGS

SEQ ID NO: 31 - protein, 348 aa, Noccaea caerulescens
PTSNLSLISIIHYRPINTLQPQMATSNGEKSLIVSFGEMLIDFVPTVSGVSLADAPGFIKAPGGAPANVAIAISRLGGRSAFVGKLGDDEFGHMLAGILKQNGVSAEGINFDTGARTALAFVTLRSDGEREFMFYRNPSADMLLRPDELNLDLIRSAKVFHYGSISLIVEPCRSAHLKAMEVAKEAGALLSYDPNLRLPLWPSKEEAQKQILSIWDKAEVIKVSDEELMFLTGSDKVDDETALSLWHSNLKLLLVTLGDKGCRYYTKSFRGSVDPFHVNAVDTTGAGDSYVGALLCKIVDDRAVLEDEARLREVLRFANACGAITTTKKGAIPALPTESEVQSLLKGN

SEQ ID NO: 32 - protein, 326 aa, Brassica rapa
MATSTGEKSLIVSFGEMLIDFVPTVSGVSLAEAPGFIKAPGGAPANVAIAVSRLGGRAAFVGKLGDDEFGHMLAGILKQNGVSAEGINFDTGARTALAFVTLRSDGEREFMFYRNPSADMLLRPDELNLDLIKSAKVFHYGSISLIVEPCRSAHLKAMEVAKEAGALLSYDPNLRLPLWPSEEEAKKQILSIWDKAEVIKVSDEELMFLTGSDNVDDETALSLWHDNLKLLLVTLGEKGCNYYTKSFRGSVDPFHVNAVDTTGAGDSYVGALLCNIVDDRAVLEDEARLREVLRFANACGAITTTKKGAIPALPTESEVQSLLNGN

SEQ ID NO: 33 - protein, 326 aa, Brassica oleracea
MATSNGEKSLIVSFGEMLIDFVPTVSGVSLAEAPGFIKAPGGAPANVAIAVSRLGGRAAFVGKLGDDEFGHMLAGILKQNGVSAEGINFDTGARTALAFVTLRSDGEREFMFYRNPSADMLLRPDELNLDLIKSAKVFHYGSISLIVEPCRSAHLKAMEVAKEAGALLSYDPNLRLPLWPSKEEAKKQILSIWDKAEVIKVSDEELMFLTGSDNVDDETALSLWHSNLKLLLVTLGEKGCNYYTKSFRGSVDPFHVNAVDTTGAGDSYVGALLCNIVDDRSVLEDEARLREVLRFANACGAITTTKKGAIPALPTESEVQSLLNGN

SEQ ID NO: 34 - protein, 326 aa, Raphanus sativus
MATFNGEKSLIVSFGEMLIDFVPTVSGVSLSEAPGFIKAPGGAPANVAIAVSRLGGRAAFVGKLGDDEFGHMLAGILKQNGVSAEGINFDTGARTALAFVTLRSDGEREFMFYRNPSADMLLRPDELNLDLIRSAKVFHYGSISLIVEPCRSAHLKAMEVAKEAGALLSYDPNLRLPLWPSKEEAKKQILSIWDKAEVIKVSDEELMFLTGSDNVDDETALSLWHDNLKLLLVTLGEKGCNYYTKSFRGSVDPFHVNAVDTTGAGDSYVGALLCNIVDDRSVLEDEARLREVLKFANACGAITTTKKGAIPALPTVSEVQTLLNAN

SEQ ID NO: 35 - protein, 329 aa, Citrus sinensis
MPTDGAVSSSSGLIVSFGEMLIDFVPTVSGVSLAEAPGFLKAPGGAPANVAIAVARLGGKAAFVGKLGDDEFGHMLAGILKENGVSADGINFDQGARTALAFVTLRADGEREFMFYRNPSADMLLRPDELNLELIRSAKVFHYGSISLIVEPCRSAHLEAMKAAKEAGALLSYDPNLRLPLWPSPEEAREQIMSIWDKAEVIKVSDVELEFLTGSDKIDDESALSLWHPNLKLLLVTLGEHGCRYYTKSFRGAVDAFHVNTVDTTGAGDAFVGALLGKLVDDQSVLEDEPRLREILKFANACGAITTTKKGAIPALPTESEALNLLKGA

SEQ ID NO: 36 - protein, 331 aa, Ziziphus jujuba
MSPVNNDVPVTGKGLIVSFGEMLIDFVPTVSGVSLAEAPGFLKAPGGAPANVAIAVSRLGGKAAFVGKLGDDEFGHMLAGILKENGVSAEGINFDQGARTALAFVTLRADGEREFMFYRNPSADMLLRPEELNLELIRSANVFHYGSISLIVEPCRSAHLKAIEVAKDAGALLSYDPNLRLPLWPSPEEAREQILSIWDKAEVIKVSDVELEFLTGSDKIDDASALSLWHDNLKLLLVTLGEKGCRYYTKHFHGEVEAFHVNTVDTTGAGDSFVGALLGKIVDDRSILEDEARLRQVLKFANACGAITTTKKGAIPALPNEPEVLALIKGA

SEQ ID NO: 37 - protein, 330 aa, Vitis vinifera
MAPVIGVNGGAGGLILSFGEMLIDFVPTVSGVSLAEAPGFLKAPGGAPANVAIAVTRLGGRSAFVGKLGDDEFGHMLAGILKENGVRGDGITFDVGARTALAFVTLRADGEREFMFYRNPSADMLLKPEELNLELIRSAKVFHYGSISLIVEPCRSAHLRAMEVAKEAGALLSYDPNLRLPLWPSAEEAREQIMSIWEKAEVIKVSDVELEFLTGSDKVDDETALSLWHPGLKLFLVTLGEKGCRYYTKNFHGAVEGFHVKTVDTTGAGDSFVGALLCKIVDDQSVLEDEARLREVLKFANACGAITTTKKGAIPALPAEADVLSLLHGA

SEQ ID NO: 38 - protein, 328 aa, Populus euphratica
MASNGVNDKSLIVSFGEMLIDFVPTVSGVSLAEAPGFVKAPGGAPANVAIAVARLGGKTAFVGKLGDDEFGNMLAGILKENGVIATGINFDTGARTALAFVTLRADGEREFMFYRNPSADMLLRPEELNLELIRSAKVFHYGSISLIVEPCRSAHLQAMRVAKDAGALLSYDPNLRLPLWPSEEEAREQILSIWDEADVIKVSDNELEFLTGSDKIDDETALSLWRPNFKLLLVTLGEKGCNYYTKNFHGSVEAFHVDTVDTTGAGDAFVGALLCKIVDDQSVLEDEPRLREILRFANACGAITTTKKGAIPALPTPAAVLKLVNEGK

SEQ ID NO: 39 - protein, 323 aa, Camelina sativa
MATPLIVSFGEMLIDFVPDTSGVSLAESTGFLKAPGGAPANVACAITKLGGKSAFIGKFGDDEFGHMLVNILKKNGVNSEGVCFDTNARTALAFVTLKNDGEREFMFYRNPSADMLLKESELNKDLIKKAKIFHYGSISLISEPCRTAHMAAMKTAKDAGVLLSYDPNVRLPLWPSTEAAIEGIKSIWNEADIIKVSDDEVTFLTRGDAEKDDVVLSLMHDKLKLLIVTDGEKGCRYYTKKFKGRVPGYAVKAVDTTGAGDSFVGSFLVSLGKDGSILDDEGKLKEALAFANACGAVCTTQKGAIPALPTPSDAQQLMKSKSK

SEQ ID NO: 40 - protein, 323 aa, Brassica napus
MATPLIVSFGEMLIDFVPDTSGVSLAESTGFLKAPGGAPANVACAISKLGGKSAFIGKFGDDEFGHMLVNILKKNGVNSEGVCFDTHARTALAFVTLKKNGEREFMFYRNPSADMLLKESELNKDLIKKAKIFHYGSISLISEPCRAAHMAAMKTAKDAGVLLSYDPNVRLPLWSSTEAAIEGIKSIWNEADIIKVSDDEVTFLTRGDAEKDDVVLSLMHDKLKLLIVTDGEKGCRYYTKKFKGRVPGYSVKTVDTTGAGDSFVGAFLVSLGKDGSILDDEGKLKEALAFANACGAVCTTQKGAIPALPTPSDAQQLMRSKSK

SEQ ID NO: 41 - protein, 326 aa, Populus euphratica
MTTNPTPLIVSFGEMLIDFVPDVAGVSLAESVGFIKAPGGAPANVACAITKLGGKSAFIGKVGDDEFGHMLVDILKKNGVNSEGVCYDPHARTALAFVTLKKNGEREFMFYRNPSADMLLQESELNMGLLKQAKVFHYGSISLISEPCRSAHLTAMKAAREAGILLSYDPNVRLPLWPSSDAARDGIKSIWNEADFIKVSDDEVAFLTQGDPQKEEVVLSLWHDRLKLLVVTDGEKGCRYFTKSFKGSVPGFSVKVVDTTGAGDAFVGSLLFSVAKDTSIFDNEEKLKEALSFANACGAICTTQKGAIPALPSGSDALALVKSKAN

SEQ ID NO: 42 - protein, 327 aa, Prunus mume
MANSESPLIVAFGEMLIDFVPDTAGVSLAESTGFLKAPGGAPANVACAITKLGGKSAFVGKVGDDEFGHMLINILKKNGVNAEGVCVDTHARTALAFVTLRKDGEREFMFYRNPSADMLLKDSELNMPLIKQAKIFHYGSISLISEPCRSAHMAAMKAAKDAGIMLSYDPNVRLPLWPSADAAREGIKSIWNQADFIKVSDDEVHFLTQGDAEKEDVVLSLWHDNLKLLVITDGEKGCRYYTKKFKGKVTGFSVKAVDTTGAGDAFVGSFLLSMAKDMSIFEDETKLKEALSFSNACGAICTTQKGAIPALPTQCAALELISKSKSK

SEQ ID NO: 43 - protein, 325 aa, Solanum tuberosum
MSKPAEIVCFGEMLIDFVPDSAGVSLAESTGFLKAPGGAPANVACAITKLDGTSAFIGKVGDDEFGRMLVDILKSNGVNSEGVLFDTHARTALAFVTLKRNGEREFMFYRNPSADMLLKESELNLGLIKQAKIFHYGSISLITEPVRSAHMVAMKAAKDAGVLLSYDPNVRLPLWPSPEAAREGIRSIWNEADFIKVSDDEVNFLTQKDADKDETVMSLWHDRLKLLIVTDGEKGCRYFTKSFKGKVSGFPVKTVDTTGAGDAFVGSLLVSIAKDPSIFQDEEKLKKALKFANACGAISTTQKGAIPALPSTADAQGLMAGSKAY

SEQ ID NO: 44 - protein, 327 aa, Glycine max
MTGSADPLVISFGEMLIDFVPDTSGVSLAESCAFIKAPGGAPANVACAISKLGGNAAFVGKMGDDEFGRMLVDILRKNGVNTDGVCFDTEARTALAFVTLRKDGEREFMFYRNPSADMLLKESELNMGLIKQAKVFHYGSISLISEPCRSAHLAAMKVAREGGALLSYDPNVRLPLWPSEEAARSGIKSIWFDADFIKVSDDEVHFLTQGDPEKEDVVMTLWHDKLKMLLVTDGEKGCRYFTKNFRGRVTGFSAKVVDTTGAGDSFVGALLTAVARDPNIFHNEPKLREALTFANACGAMCTTQKGAIPALPTAAEAEKFISNSKAK

SEQ ID NO: 45 - protein, 335 aa, Zea mays
  misc_feature (297)..(297): Xaa can be any naturally occurring amino acid
  misc_feature (305)..(305): Xaa can be any naturally occurring amino acid
MAPLGDGGAAAAAASNNLVVSFGEMLIDFVPDVAGLSLAESGGFVKAPGGAPANVACAIAKLGGSSAFVGKFGDDEFGHMLVNILKQNNVNSEGCLFDKHARTALAFVTLKHDGEREFMFYRNPSADMLLTEAELDLGLVRRARVFHYGSISLISEPCRSAHMAAMRAAKSAGVLCSYDPNVRLPLWPSPDAAREGILSIWKEADFIKVSDDEVAFLTRGDANDEKNVLSLWFDGLKLLVVTDGDKGCRYFTKDFKGSVSGFKVDTVDTTGAGDAFVGSLLVNVAKDDSIFHNEEKXREALKFSXACGAICTTKKGAIPALPTVATAQDLIAKAN

SEQ ID NO: 46 - protein, 323 aa, Brassica rapa
MATPLIVSFGEMLIDFVPDTSGVSLAESTGFLKAPGGAPANVACAISKLGGKSAFIGKFGDDEFGHMLVNILKKNGVNSEGVCFDTHARTALAFVTLKKNGEREFMFYRNPSADMLLKESELNKDLIKKAKIFHYGSISLISEPCRAAHMAAMKTAKDAGVLLSYDPNVRLPLWPSTEAAIEGIKSIWNEADIIKVSDDEVTFLTRGDAEKDDVVLSLMHDKLKLLIVTDGEKGCRYYTKKFKGRVPGYSVKTVDTTGAGDSFVGAFLVSLGKDGSILDDEGKLKEALAFANACGAVCTTQKGAIPALPTPSDAQQLMKSKSK

SEQ ID NO: 47 - protein, 320 aa, Brassica oleracea
MATPLIVSFGEMLIDFVPDTSGVSLAESTGFLKAPGGAPANVACAISKLGGKSAFIGKFGDDEFGHMLVNILKKNGVNSEGVCFDTHARTALAFVTLKKNGEREFMFYRNPSADMLLKESELNKDLIKKAKIFHYGSISLISEPCRAAHMAAMKTAKDAGVLLSYDPNVRLPLWSSTEAAIEGIKSIWNEADIIKVSDDEVTFLTRGDAEKDDVVLSLMHDKLKLLIVTDGEKGCRYYTKKFKGRVPGYSVKTVDTTGAGDSFVGAFLVSLGKDGSILDDEGKLKEALAFANACGAVCTTQKGAIPALPTPSDAQQLMKS

SEQ ID NO: 48 - protein, 323 aa, Raphanus sativus
MATPLIVSFGEMLIDFVPDTSGVSLAESTGFLKAPGGAPANVACAISKLGGKSAFIGKFGDDEFGHMLVNILKKNGVNSEGVCFDSHARTALAFVTLKKNGEREFMFYRNPSADMLLKESELNKDLIKKAKIFHYGSISLISEPCRAAHMAAMKTAKDAGVLLSYDPNVRLPLWPSTEAAIEGIKSIWNEADIIKVSDDEVTLLTGGDAEKDDVVLSLMHDKLKLLIVTDGEKGCRYYTKKFKGRVPGYSVKTVDTTGAGDSFVGAFLFSLGKDGSILDDEGKLKEALAFANACGAVCTTQKGAIPALPTPSDAQQLMKSKSK

SEQ ID NO: 49 - protein, 324 aa, Citrus sinensis
MSSQLIVCFGEMLIDFVPDVSGVSLAESTGFLKAPGGAPANVACAITKLGGSSAFVGKFGDDEFGHMLVNILKQNNVKTEGVCFDNHARTALAFVTLKKNGEREFMFYRNPSADMLLKDSELNMGLIKQAKIFHYGSISLISEPCRSAHMAALKAAKDAGVLLSYDPNVRLPLWPSQDAARDGIKSIWNHADLIKVSDDEVNFLTKGGDAEKDDVVMSLWHDNLKLLLVTYGAKGCGYFTKKFKGRVPGFSVKTIDTTGAGDAFVGSFLVSVAKDANIFNDEGKLKEALIFSNACGAICTTQKGAIPALPPPDKVQELIKSNAK

SEQ ID NO: 50 - protein, 328 aa, Ziziphus jujuba
MTNSGSSPFIVSFGEMLIDFVPDTSGVSLAESSGFLKAPGGAPANVACAITKLGGQSAFIGKFGDDEFGHMLVNILKKNNVNADGVCFDEHARTALAFVTLKKNGEREFMFYRNPSADMLLKDTELNMGLIKKAKIFHYGSISLITEPCRSAHMAAMKAAKEAGVLLSYDPNVRLPLWPSAEAARDGIKSIWNHADFIKVSDDEVQFLTEKDAQKDEVVMSLWHDKLKLLVVTDGEKGCRYYTKNFKGRVSGFSVKTVDTTGAGDAFVGSLLYSMAKDFSIFTDEAKLKEALYFANACGAICTTQKGAIPALPTQSDAQGLISMSKTK

SEQ ID NO: 51 - protein, 328 aa, Vitis vinifera
MTITSKSPLVVAFGEMLIDFVPDSAGVSLAESTGFLKAPGGAPANVACAITKLGGNSAFIGKVGDDEFGHMLVDILKKNGVNSEGVCFDAHARTALAFVTLKKNGEREFMFYRNPSADMLLTESELNMGLIKQAKIFHYGSISLISEPCRSAHMAAMKAAKEAGILLSYDPNVRLPLWPSAQAAIDGIKSIWNHADFIKVSDDEVGFLTQGDAEKEDVVLSLWHDNLKLLVVTDGEKGCRYFTKGFKGRVEGFSVQTVDTTGAGDAFVGALLVSIAQDPSIFQDEGKLKEALKYANACGAICTTQKGAIPALPTNSDALDLVNKSKAK

SEQ ID NO: 52 - protein, 325 aa, Nicotiana sylvestris
MAKSAEIVCFGEMLIDFVPDSAGVSLAESTGFLKAPGGAPANVACAITKLDGTSAFIGKVGDDEFGRMLVDILKSNGVNSEGVCFDTQARTALAFVTLKSNGEREFMFYRNPSADMLLKESELNLGLIKQAKIFHYGSISLITEPCRSAHMAAMKAAKEAGVLLSYDPNVRLPLWPSPEAARDNIRSIWNEADFIKVSDDEVNFLTQKDAEKEETVLSLWHDRLKLLVVTDGEKGCRYFTKSFKGKVSGFSVKTIDTTGAGDAFVGSLLVSIAKDPSIFQDEEKLKKALRFANACGAISTTQKGAIPALPSTSDAQGLIAGSKAY

SEQ ID NO: 53 - protein, 327 aa, Cicer arietinum
MSGSKDPLVISFGEMLIDFVPDTSGVSLAESHAFIKAPGGAPANVACAVSKLGGNAAFIGKVGGDEFGRMLVDILKKNGVNTDGVCFDTEARTALAFVTLRKDGEREFMFYRNPSADMLLTESELNMGLIKQAKIFHYGSISLISEPCRSAHMAAMNAAREGGALLSYDPNVRLPLWSSPDAARSGIKSIWFDADFIKMSDDEVEFLTQKDPKNEDVIMSLWHDKLKMLLITDGEKGCRYVTKNFKGRVSGFSVKAVDTTGAGDSFVGALLTAVARDTSIFDNEPKLRETLTFANACGAMCTTQKGAIPALPTAEEAQKFISSSKAN

SEQ ID NO: 54 - protein, 324 aa, Citrus clementina
MSSQLIVCFGEMLIDFVPHVSGVSLAESTGFLKAPGGAPANVACAITKLGGSSAFVGKFGDDEFGHMLVNILKQNNVKTEGVCFDNHARTALAFVTLKKNGEREFMFYRNPSADMLLKDSELNMGLIKQAKIFHYGSISLISEPCRSAHMAALKAAKDAGVLLSYDPNVRLPLWPSQDAARDGIKSIWNHADLIKVSDDEVNFLTKGGDAEKDDVVMSLWHDNLKLLLVTYGAKGCGYFTKKFKGRVPGFSVKTIDTTGAGDAFVGSFLVSVAKDANIFNDEGKLKEALIFSNACGAICTTQKGAIPALPPPDKVQELIKSNAK

SEQ ID NO: 55 - protein, 344 aa, Juglans regia
MTISATSPSPSIVAFGEMLIDFVPDMAGVSVGTKGLWPRSVSLAESTAFLKAPGGAPANVACAITKLGGNSAFIGKLGDDEFGHMLVDILKKNEVNTDGVCFDPDARTALAFVTLKKNGEREFMFYRNPSADMLLKESELNIGLIKRAKIFHYGSISLISEPCRSAHIAAMKAAKEAGILLSYDPNVRLPLWPSADAARDGIKSIWDQADFIKVSDEEVQFLTDGDPGKEDVVLRLWHDNLKLLVVTDGEKGCRYFTKSFKGKVAGFSVNTVDTTGAGDAFVGALLVSCMANDSISCIFHDEGKLREALTFANACGAICTTKKGAIPALPSTFQAHELINKSKA

SEQ ID NO: 56 - protein, 323 aa, Fragaria vesca
MADSLIVAFGEMLIDFVPDTAGVSLAESTGFLKAPGGAPANVACAITKLGGKSAFVGKVGDDEFGHMLINILKTNGVNTEGVCVDTHARTALAFVTLRKDGEREFMFYRNPSADMLLKDSELNMDLIKKAKIFHYGSISLISEPCKSAHMAAMKAAKDAGILLSYDPNVRLPLWPSADAARDGIKSIWNQADFIKVSDDEVQFLTQGDPNKEEVVLSLWHDNLKLLIVTDGEKGCRYFTKKFKGNVTGFAVKTVDTTGAGDAFVGSFLLSMAKDLSIFEDEAKLTEALSFANACGAICTTQKGAIPALPAESDALELIKSAAK

SEQ ID NO: 57 - protein, 329 aa, Ricinus communis
MTISTANPAPLIVSFGEMLIDFVPDVAGVSLAESGAFIKAPGGAPANVACAITKLGGNSAFIGKVGEDEFGHMLVNILKKNGVNCEGVSFDQEARTALAFVTLKKNGEREFMFYRNPSADMLLKESELNMGLIKQAKIFHYGSISLISEPCRSAHMAAMKAAKAAGILLSYDPNVRLPLWPSHEAARDGIKSIWNEADFIKVSDDEVAFLTQGDPTKEEVVLSLYHDRLKLLIVTDGEKGCRYFTKSFKGKVSGYSVKTVDTTGAGDAFVGALLVSVAKDASIFENEGKLKEALTFANACGAICTTQKGAIPALPSTSDAQALIKSSKA

SEQ ID NO: 58 - protein, 325 aa, Jatropha curcas
MTNSSPLIVCFGEMLIDFVPDVAGVSLAESGGFIKAPGGAPANVACAITKLGGNSAFIGKFGDDEFGHMLVNISKKNGVNSEGVRFDENARTALAFVTLKKNGEREFMFYRNPSADMLLKESELNLDLIKQAKIFHYGSISLITEPCRSAHMAAMKAAKAAGVLLSYDPNLRLPLWPSSDAARKGIKSIWKEADFIKVSDEEVAFLTQGDPSKEEVALQLWHEGLKLLVVTDGEKGCRYFTKSFKGKVAGFSVKTVDTTGAGDSFVGSLLCSVAKDTSIFDNEEKLKEALTFANACGAICTTQKGAIPALPSPSDAQALIKSKSN

SEQ ID NO: 59 - protein, 329 aa, Malus domestica
MANSVSPLIVAFGEMLIDFVPDTAGVSLAESTGFLKAAGGAPANVACAITKLGGKSAFVGKVGDDEFGHMLINILKKNGVNAEGVCVDKHARTALAFVTLKNDGEREFMFYRNPSADMLLKDSELNMGLIKQAKIFHYGSISLIAEPCRSAHMAAMKAAKEAGVLLSYDPNVRLPLWPSADAARQEIKSIWNQADFIKVSDDEVNFLTQGDSDKEDVVLSLWHDNLKLLVVTDGEKGCRYYTKKFKGKVAGFSVKAVDTTGAGDSFVGSFLLSMANDMSIFEDEAKLKEALSFANACGAICTTQKGAIPEFSVFTKLFNYFYKIHLHFY

SEQ ID NO: 60 - protein, 325 aa, Sesamum indicum
MTTKSLIVCFGEMLIDFVPDTAGVSLAESHGFLKAPGGAPANVACAITKLSGSSAFIGKVGEDEFGRMLVDILKKNGVNSEGVRFDQQARTALAFVTLKHNGDREFMFYRNPSADMLLKESELDIKMIKQASIFHYGSISLISEPCRSAHMAAMKDAKEAGALLSYDPNVRLPLWPSAQAARDGIKSIWNKADFIKVSDDEVEFLTQKDPQKEDAVMSLWHDQLKLLVITDGGKGCRYFTKSFKGKVDGFSVKTIDTTGAGDAFVGALLVSIAKNPSIMQDEAKLKEALTFANACGAISTTRKGAIPALPTVSDAQALIAQAKAK

SEQ ID NO: 61 - protein, 358 aa, Theobroma cacao
MANSFASLNPQLIVSFGEMLIDFVPDVAGASLAESSAFLKAPGGAPANVACAIKKLGGNSAFIGKFGDDEFGHMLVDVLKKHGVNTDGICFDPHARTALAFVTLKRNGEREFTFYRNPSADMLLKESDLKISMIKQAKIFHYGSISLISEPCRSAHMAAMKAAGEAGVFLSYDPNVRLPLWPSPQAARDGILSIWNYADFIKVSDDEVAFLTQGDPGREDVVLSLWHQNLKLLIVTDGEKGCRYFTKHFKGKVDGFSVKTVDTTGAGDAFVGSILVSLAMDTKFFHDEGRLREALLFANACGAICVTKKGAIPALPTRSDALGLIKSKAMPLLTKTCMLFSSLAALVSILVFLLYKPK

SEQ ID NO: 62 - protein, 344 aa, Juglans regia; identical to SEQ ID NO: 55

SEQ ID NO: 63 - protein, 335 aa, Ananas comosus
MAIGEAEKTKGSSGEEALVVSFGEMLIDFVPDAAGVSLAESTGFLKAPGGAPANVAVAIAKLGGSAAFIGKFGDDEFGHMLVEILRQNGVRDEGVLFDKDARTALAFVTLRSDGEREFMFYRNPSADMLLAESELNLPLIRQARIFHYGSISLISEPCRSAHLAAMRAAKAAGILCSYDPNVRLPLWPSPDAARSGIMSIWNQADFIKVSDDEVEFLTQRDPQSEENVLSLWYEGLKLLIVTDGEKGCRYFTKSFKGRVPGFSVKTVDTTGAGDAFVGALLVSIAKDGSLFQNEEKLREALKFANACGAICTTQKGAIPALPTTATAMELVSKGK

SEQ ID NO: 64 - protein, 327 aa, Gossypium raimondii
MNNNSAKSPMIVSFGEMLIDFVPDVAGVSLAESYAFIKAPGGAPANVACAIAKLGGNAAFIGKVGDDEFGHMLANLLKKSGVNSDGICFDKDARTALAFVTLKADGQREFMFYRSPSADMLLKESELKLDLIKQAKIFHYGSISLISEPCKSAHMAAMKAAKQAGVLLSYDPNVRLPLWPSPEAARDGIKSIWDQADFVKVSDDEVEFLTKGDPKKDDVVMSLWNDNFKLLIVTDGPEGCRYFTKKFKGKVNGYKVKTIDTTGAGDAFVGAFLQAVAKDPNLFNDENKLKEALVFANACGAISTTQKGAIPSLPDKAQAENLIKEAK

SEQ ID NO: 65 - protein, 389 aa, Solanum lycopersicum
MALHATAFSFTGVSTSSKSSRSALLSVFPLPRRCTVKATSQYPHSFPRCKIQGRALPSDNGLVEKDESSLVVCFGEMLIDFVPTTSGLSLAEAPAFKKAPGGAPANVAVGISRLGGSSAFIGKVGEDEFGYMLAEILKENNVNSDGMRFDPGARTALAFVTLRKDGEREFMFYRNPSADMLLQEDELDLELIRKAKVFHYGSISLITEPCKSAHIAAAKAAKDAGVILSYDPNLRLPLWPSAESAREGILSIWNTADIIKISEEEISFLTQGEDPYDDNVVRKLYHPNLKLLLVTEGPEGCRYYTKDFSGRVKGIKVDAVDTTGAGDAFVAGILSQLASDVSLLQDEGKLRDALSFANACGALTVMERGAIPALPTKEVVLNALLKSVA

SEQ ID NO: 66 - protein, 386 aa, Camelina sativa
MALQAASTFCFSGPTFRSTPYSLTSNRRSISIKATSSSSSSSRISNSRSNLKGRALSSEGSTQESPDVVCFGEMLIDFVPTTSGLSLADAPAFKKAPGGAPANVAVGIARLGGSSAFIGKVGEDEFGYMLANILKDNNVNNEGMRFDPGARTALAFVTLTSEGEREFMFYRNPSADMLLEESELDLDLIKKAKIFHYGSISLITEPCKSAHIAASKAAKEAGVILSYDPNLRLPLWPSADNARDEILSIWDTADIIKISEEEIVFLTKGEDPYDDNVVRKLFHPKLKLLLVTEGPEGCRYYTKDFSGRVHGLKVDVVDTTGAGDAFVAGILSQLANDLSLLQDEERLREALMFANACGALTVKQRGAIPALPTREAVHEALLQSVV

SEQ ID NO: 67 - protein, 391 aa, Brassica napus
MALQATTTTFSFSAPTFRSTPPHALTSKRRLSIKASSSSSPGLSSPPLLSLSRSNLKGRAFASDGSTQESPSVVCFGEMLIDFVPTTSGLSLAQAPAFKKAPGGAPANVAVGIARLGGSSAFIGKVGEDEFGYMLANILKDNNVNNEGMRFDPGARTALAFVTLTNEGEREFMFYRNPSADMLLEESELDLDLIKKAKIFHYGSISLITEPCKSAHIAAAKAAKEAGVILSYDPNLRLPLWPSADNARDEILSVWDTADIIKISEEEIEFLTKGEDPYDDSVVRKLFHPKLKLLLVTEGPEGCRYYTKDFSGRVHGLKVEVVDTTGAGDAFVAGILSQLACDLSLLQDEERLREALMFANACGALTVKERGAIPALPTKEAVLEALLKAVV

SEQ ID NO: 68 - protein, 384 aa, Populus euphratica
MAALHSTAFCLGGVALSSRQEICFRRGNVKASTFSCPPLVSIPRLSFQSKAFGGDGSPETKDSSLVVCFGEMLIDFVPTISGLSLSDAPAFKKAPGGAPANVAVGIARLGGSSAFIGKVGEDEFGYMLAEILKENNVNNEGMRFDPGARTALAFVTLRSDGEREFMFYRNPSADMLLQEAELDLDLIRKAKIFHYGSISLITEPCKSAHIAAAKVAKDAGVVLSYDPNLRLPLWPSAESAREGILSIWDTADIIKISEEEISFLTKGEDPYDDAVVQKLFHPNLKLLLVTEGPEGCRYYTKEFRGRVKGLKVDAVDTTGAGDAFVAGILSQLAKDLSLLQNEDRLREALKFANACGALTVKERGAIPALPTREAVNSAIIQLVG

SEQ ID NO: 69 - protein, 383 aa, Prunus mume
MALYSTAFCFSGVVSLPRNSVCFSQRTVRASAFSCPPLSSISRSNVQGKAFAGDGLLDTKESSLVVCFGEMLIDFVPTTNGLSLAEAPAFKKAAGGAPANVAVGIARLGGSSAFIGKVGEDEFGYMLADILKENNVNSEGMRFDPGARTALAFVTLRSDGEREFMFYRNPSADMLLQEAELDFDLIRKAKILHYGSISLITEPCKSAHIAAAKSAKDAGVVLSYDPNLRLPLWPSAKSAREGILSIWEIADVIKVSEEEISFLTEGEDPYDENVVRKLYHPNLKLLLVTEGPDGCRYYTKEFSGRVKGMKVDAVDTTGAGDAFVAGILSQLAVDLSLLQEEGKLRDALLFANACGALTVTERGAIPALPTREAVLNVMLKSVS

SEQ ID NO: 70 - protein, 386 aa, Malus domestica
  misc_feature (44)..(44): Xaa can be any naturally occurring amino acid
MALHSTAFCFGGVVSLPRNSVSFSQRTVRASAFASPPPLSSSSXARSNVQGKAFAGDVLSDTKESSLVVCFGEMLIDFVPTTNGLSLAEAPAFKKAAGGAPANVAVGIARLGGSSAFIGKVGEDEFGYMLADILKENNVNNEGLRFDPGARTALAFVTLRSDGEREFMFYRNPSADMLLQEAELDFDLIRKAKILHYGSISLITEPCKSAHIAATKAAKDAGVVLSYDPNLRLPLWPSAKSAREGILSIWDSADVIKISEEEISFLTEGEDPYDENVVRKLYHPNLKLLLVTEGPDGCRYYTKEFSGRVKGMKVDVVDTTGAGDAFVAGILSQLAVDLSLLQDEDKLRDALLFANACGALTVMGRGAIPALPTRESVLNVLLKSVA

SEQ ID NO: 71 - protein, 394 aa, Capsicum annuum
MALHATAFSFTGVSTSNQTYRNSLPSVFPAPRRCTVKAAAAPFPSQFPPILSRCKIQGRALPSDDGPLKKDESSLVVCFGEMLIDFVPTTSGLSLAEAPAFKKAPGGAPANVAVGISRLGGSSAFIGKVGEDEFGYMLADILKENNVNSDGMRFDPGARTALAFVTLRKDGEREFMFYRNPSADMLLQEDELDLELIKKAKVFHYGSISLITEPCKSAHIAAARAAKDAGVILSYDPNLRLPLWPSAESAREGILSIWDTADIIKISEEEISFLTQGEDPYDDNVVRKLYHPNLKLLLVTEGPEGCRYYTKDFSGRVKGIKVDAVDTTGAGDAFVAGILSQLASDVSLLQDESKLRDALSFANACGALTVMERGAIPALPTREVVLNTLVKSVA

SEQ ID NO: 72 - protein, 384 aa, Cucumis melo
MALYSGAFCFHGVPTVTLESLRHKEWAVKSYGLSRSRVVSKSQLKVKVKAFSGDGGSVESNDSSLVVCFGEMLIDFVPTISGLSLAEAPAFKKAPGGAPANVAVGIARLGGSSAFIGKVGADEFGYMLADILKENNVCNEGMLFDPGARTALAFVTLRSDGEREFMFYRNPSADMLLQESELNYDLIRKAKIFHYGSISLITEPCKSAHIAAAKTAKDAGAVLSYDPNLRLPLWPSADSAIEGILSIWDTADIIKISEEEISFLTRGEDPFDDAVVRKLFHPNLKLLLVTEGPGGCRYYTKEFSGRVKGLKVDAVDTTGAGDAFVAGILSQLATDLSLLEKEDKLRDALLFANACGALTVKERGAIPALPNKDAVLNAIFKTVA

SEQ ID NO: 73 - protein, 383 aa, Capsella rubella
MMALQAATSMFCFSGPTLRSNSLTSSRRSISIKASSSKLSNSRSNLLKGRALSSSDGSTQESPDVVCFGEMLIDFVPTTSGLSLADAPAFKKAPGGAPANVAVGIARLGGSSAFIGKVGEDEFGYMLASILKDNNVNNEGMRFDPGARTALAFVTLTSEGEREFMFYRNPSADMLLEESELDLDLIKKAKIFHYGSISLITEPCKSAHIAAAKAAKEAGVLLSYDPNLRLPLWPSPDNARDEILSIWDTADIIKISEEEIVFLTKGEDPYDDNVVRKLFHPKLKLLLVTEGPEGCRYYTKDFNGRVHGLKVDVVDTTGAGDAFVAGILSQLANDLSLLQDEERLKEALMFANACGALTVKQRGAIPALPTKEAVHEALLQSVV

SEQ ID NO: 74 - protein, 390 aa, Noccaea caerulescens
MALQATTTFCFSGPTLRSNPPDSLTSKRRFSSIKASSTSSGLSSPPLLSLSRSNLKGRAFSSDGSTQESPYVVCFGEMLIDFVPTTSGLSLADAPAFKKAPGGAPANVAVGIARLGGSSAFIGKVGEDEFGYMLANILKDNNVNNEGMRFDPGARTALAFVTLTSEGEREFMFYRNPSADMLLQESELDLDLIKKAKIFHYGSISLITEPCKSAHIAAAKAAKEAGVILSYDPNLRLPLWPSAENARDEILSIWDTADIIKISEEEIEFLTKGEDPYDDSVVRKLFHPNLKLLLVTEGPEGCRYYTKDFSGRVRGLKVEVVDTTGAGDAFVAGILSQLSLDLSLLQDEERLREALLFANACGALTVKERGAIPALPTKEAVLEALLKPVI

SEQ ID NO: 75 - protein, 388 aa, Brassica rapa
MALQATTSTFSFSAPTFRSTPPHALTSKRRLSITASSSGLSSPPLLSLSRSNLKGRAFASDGSTQESPSVVCFGEMLIDFVPTTSGLSLAQAPAFKKAPGGAPANVAVGIARLGGSSAFIGKVGEDEFGYMLANILKDNNVNNEGMRFDPGARTALAFVTLTNEGEREFMFYRNPSADMLLEESELDLDLIKKAKIFHYGSISLITEPCKSAHIAAAKAAKEAGVILSYDPNLRLPLWPSADNARDEILSVWDTADIIKISEEEIEFLTKGEDPYDDSVVRKLFHPKLKLLLVTEGPEGCRYYTKDFSGRVHGLKVEVVDTTGAGDAFVAGILSQLACDLSLLQDEERLREALMFANACGALTVKERGAIPALPTKEAVLEALLKAVV

SEQ ID NO: 76 - protein, 389 aa, Brassica oleracea
MALQATTTTFSFSAPTFRSTPRALTSKRRLSITASSSSPGLSSPPLLSLSRSNLKGRAFASDGSTQESPSVVCFGEMLIDFVPTTSGLSLAQAPAFKKAPGGAPANVAVGIARLGGSSAFIGKVGEDEFGYMLANILKENNVNNEGMRFDPGARTALAFVTLTNEGEREFMFYRNPSADMLLEESELDLDLIKQAKIFHYGSISLITEPCKSAHIAAAKAAKEAGVILSYDPNLRLPLWPSADNARDEILSVWDTADIIKISEEEIEFLTKGEDPYDDSVVRKLFHPKLKLLLVTEGPEGCRYYTKDFSGRVHGLKVEVVDTTGAGDAFVAGILSQLACDLSLLQDEERLREALMFANACGALTVKERGAIPALPTKEAVLDALLKAVV

SEQ ID NO: 77 - protein, 389 aa, Brassica napus
MALQATTSTFSFSAPTFRSTPPHALTSKCRLSIKASSSSGLSSPPLLSLSRSNLKGRAFASDGSTQESPSVVCFGEMLIDFVPTTSGLSLAQAPAFKKAPGGAPANVAVGIARLGGSSAFIGKVGEDEFGYMLANILKDNNVNNEGMRFDPGARTALAFVTLTNEGEREFMFYRNPSADMLLEESELDLDLIKKAKIFHYGSISLITEPCKSAHIAAAKAAKEAGVILSYDPNLRLPLWPSADNARDEILSVWDTADIIKISEEEIEFLTKGEDPYDDSVVRKLFHPKLKLLLVTEGPEGCRYYTKDFSGRVHGLKVEVVDTTGAGDAFVAGILSQLACDLSLLQDEERLREALMFANACGALTVKERGAIPALPTKEAVLEALLKAVV

SEQ ID NO: 78 - protein, 393 aa, Raphanus sativus
MALQATTTTFSFSAPTFRSTPPPPHAFTSKRRLSIKSSSSGLSSPPPPLLSLSRSNLKGRAFSSDGSTTQESPSVVCFGEMLIDFVPTTSGLSLAQAPAFKKAPGGAPANVAVGIARLGGSSAFIGKVGEDEFGYMLANILKDNNVNNEGMRFDPGARTALAFVTLTSEGEREFMFYRNPSADMLLEESELDLDLIKKAKIFHYGSISLITEPCKSAHIAAAKAAKEAGVILSYDPNLRLPLWPSADSARDEILSVWDTADIIKISEEEIEFLTKGEDPYDDSVVRKLFHPKLKLLLVTEGPEGCRYYTKDFSGRVHGLKVEVVDTTGAGDAFVAGILSQLACDLSLLQDEERLREALMFANACGALTVKERGAIPALPTKQAVLDALLKAVV

SEQ ID NO: 79 - protein, 386 aa, Pyrus x bretschneideri
MALHSTAFCFGGVVSLPRNSVSFSQRTVRASAFASPPPLSSSSIARLNVQGKAFAGDVLLDTKESSLVVCFGEMLIDFVPTASGLSLAEAPAFKKAAGGAPANVAVGIARLGGSSAFIGKVGEDEFGYMLADILKENNVNNEGMRFDPGARTALAFVTLRSDGEREFMFYRNPSADMLLQEAELDFDLIRKAKILHYGSISLITEPCKSAHIAATKAAKDAGVVLSYDPNLRLPLWPSAKSAREGILSIWDTADVIKISEEEISFLTEGEDPYDENVVRKLYHPNLKLLLVTEGPDGCRYYTKEFSGRVKGMKVDAVDTTGAGDAFVAGILSQLAVDLSLLQDEDKLRDALVFANACGALTVTGRGAIPALPTRESVLNVLLKSVA

SEQ ID NO: 80 - protein, 385 aa, Cicer arietinum
MALHCASFGFTTVPSNPPRSVNFSQSSVKTSVFPSPLSLSNIRLKVQGKTKAMTGNGTPETKESPLVVCFGEMLIDFVPTISGVSLADAPAFKKAPGGAPANVAVGISRLGGSSAFIGKVGEDEFGYMLADILKENNVNKQGMRFDPGARTALAFVTLRSDGEREFMFYRNPSADMLLQEDELDLDLIRKAKIFHYGSISLITEPCKSAHIAATKAAKEAGVVLSYDPNLRLPLWPSAESAREGILSIWDTADIIKISEEEISFLTNGEDPYDDTVVRKLFHSNLKLLLVTEGAEGCRYYTKEFSGRVKGMKVDAVDTTGAGDAFVAGILSQLASDLSLLQKEEQLRDSLKFANACGALTVTERGAIPALPTKETVLNAILKPVS

SEQ ID NO: 81 - protein, 391 aa, Sesamum indicum
MALHCAAFSFNSVSLSPQASGPSLRFLSPSSFLWKKSVKASVFSSLSPARFRLQGKALESGNGPLKSDESPLVVCFGEMLIDFVPTTSGLSLAEAPAFKKAPGGAPANVAVGIARLGGSSAFIGKVGEDEFGYMLADILRENNVNNKGMRFDPGARTALAFVTLRKDGEREFMFYRNPSADMLLQESELDLDLIKKAKIFHYGSISLITEPCKSAHIAAAKAAKDAGVILSYDPNLRLPLWPSAESAREGILSIWDTADIIKISEEEICFLTQGEDPYDDNVVRKLYHQNLKLLLVTEGPDGCRYYTKEFSGRVKGLKVEPVDTTGAGDAFVAGILSQLASDTSLLQDEGRLRDALRFANACGALTVTERGAIPALPTRETVHSALLKSVA

SEQ ID NO: 82 - protein, 382 aa, Jatropha curcas
MALLLHSSAFCFSGVVHSVTLKRCNARASVGFSAPPLVSIPRFSLQGKAFSGDRLPETKDSSLVVCFGEMLIDFVPTVNGLSLADAPAFKKAPGGAPANVAVGIARLGGSSAFIGKVGEDEFGYMLADILKENNVNNEGMRFDPGARTALAFVTLRSDGEREFMFYRNPSADMLLQEAEVDFNLIRKAKIFHYGSISLITEPCKSAHIAAAKAAKDAGVLLSYDPNLRLPLWPSADSAREGILSIWELADIIKISEEEISFLTKGEDPYDDAVVRKFFHPNLRLLLVTEGADGCRYYTKEFSGRVKGLKVDAIDATGAGDAFVAGILSQLAEDFSLLQNEERLRDALKFANACGALTVMDRGAIPALPTREAVLNAILKSVA

SEQ ID NO: 83 - protein, 388 aa, Gossypium raimondii
MAALYSTSSCFCGSLGSYPVHKSVKLGHGKVRDSGFYFPHRFQCATPKLHVQAKALSVDGLPEIMDSSLVVCFGEMLIDFVPTISGLSLAEAPAFKKAPGGAPANVAVGIARLGGSSAFIGKVGEDEFGYMLADILKENNVNNEGMRFDPGARTALAFVTLRSDGEREFMFYRNPSADMLLQENELDFDLITKATIFHYGSISLITEPCKSAHIAAAKVAKDAGVVLSYDPNLRLPLWPSAESARKGILSIWDTADIIKVSEEEISFLTQGEDPYDDAVVRKLFHPNLKLLLVTEGADGCRYYTQEFSGKVKGLKVEAVDTTGAGDAFVAGTLSQLASDLSLIQDEDRLRNALKFSNVCGALTVTERGAIPALPTREAVVNALLKTVA

SEQ ID NO: 84 - protein, 383 aa, Eucalyptus grandis
MALHSSAACFNGVGISRRDFVSCRRRTVKVSAFSCPPLSSIARLNVKGKAVRGNDAPETKDNSLVVCFGEMLIDFVPTVSGLSLAESPAFKKAPGGAPANVAVGISRLGGSSAFIGKVGEDEFGYMLADILKENNVNSEGMRFDPGARTALAFVTLRNDGEREFMFYRNPSADMLLEESELDYDLIRKAKILHYGSISLITEPCKSAHIAAAKAAKDAGVILSYDPNLRLPLWPSAESAREGILSIWEIADIIKISEEEISFLTKGEDPYDDAVVRKLYHPNLKLLLVTEGPDGCRYYTKDFSGRVKGLKVDAVDTTGAGDAFVAGILSQLAVDLSLLQEEDRLRDALKFANACGALTVTERGAIPALPTREVVLNTLLKSVA

SEQ ID NO: 85 - protein, 347 aa, Solanum lycopersicum
MAGESISGNLKDLSLNRNGAVSKKSHLVVCFGEMLIDFIPTVAGVSLAEAPAFEKAPGGAPANVAVCISKLGGSSAFIGKVGDDEFGRMLADILKQNNVDNSGMRFDHDARTALAFITLTAEGEREFVFFRNPSADMLLRESELDVDLIKKATIFHYGSISLIDEPCRSTHLAAMDIAKRSGSILSYDPNLRLPLWPSEDAARSGIMSVWNLADIIKISEDEISFLTGADDPNDDEVVLKRLFHPNLKLLLVTEGSAGCRYYTKEFKGRVNSIKVKAVDTTGAGDAFTGGVLKCLASDASLYQDEKRLREAIFFANVCAALTVTGRGGIPSLPTQDAVRQTLAEVTA

SEQ ID NO: 86 - protein, 343 aa, Brassica napus
MGENAISGNLKNLKIDTGRSETLVVCFGEMLIDFVPTVGGVSLAEAPAFKKAPGGAPANVAVGVSRLGGSSAFVGKVGDDEFGRMLADILRLNNVDNSGMRFDHKARTALTFVTLRGDGEREFLFFRHPSADMRLLESELDKNLIQKAKIFHYGSIGLIEEPCRSTQLAAMKIAKASGSLLSYDPNLRLPLWPSEEAARKEIMSIWNLADVIKISEEEITFLTGGDDPYDDEVVLRKLFHPNLKLLVVSEGPNGCRYYTQEFKGRVGGVKVKAVDTTGAGDAFVSGLLNSLASDLTLLTDEKKLREALLFANACGAITATERGAIPAMPTMDAVQDLLSSSRS

SEQ ID NO: 87 - protein, 358 aa, Prunus mume
MANNLTSALDKKLVLGEDSKDPSSDAKGGSKDDKSLVVCFGEMLIDFVPTVGGVSLAEAPAFKKAPGGAPANVAVGVSRLGGSSGFIGKVGDDEFGYMLADILKQNNVNNSGLRFDPSARTALAFVTLRADGEREFLFFRNPSADMLLRESELDINLIQQARIFHYGSISLIDEPCRSTHLAAMRIAKKSGCILSYDPNLRLPLWPSEEAARKGIMSIWDQADIIKISEDEITFLTGGDDPYDDNVVLTKLFHPNLKLLVVTEGSEGCRYYTQKFRGRVAGVKVKPVDTTGAGDAFVSGVLNSIASDLSLFQNEQGLREALLFANACGALTVTERGAIPAMPTREAVLRCLAQVADKK

SEQ ID NO: 88 - protein, 358 aa, Malus domestica
MANNLGPALDKKLVLCEDSKDASSDKKGGSQDDKSLVVCFGEMLIDFVPTVGGVSLAEAPAFNKAPGGAPANVAVGVARLGGSAGFIGKVGDDEFGYMLADILKQNNVDNSGVRFDPSARTALAFVTLRADGEREFLFFRHPSADMLLRESELDINLIKKAKIFHYGSISLIDEPCRSTHLAAMKIAKKSGCILSYDPNLRLPLWPSEDAALKGIMSIWDQADIIKISEDEIRFLTGGDDPCDDNVVLTKLFHPNLRLLVVTEGSEGCRYYTQKFHGRVGGVKVKPVDTTGAGDAFVGGVLNSIASNLSLFQDEKGLREALLFANACGALTVTERGAIPAMPTREAVLQCLKQAAEKK

SEQ ID NO: 89 - protein, 346 aa, Capsella rubella
MGENAISGNLKNLTIDTRDSETLVVCFGEMLIDFVPTVGGVSLAEAPAFKKAPGGAPANVAVGVSRLGGSSAFIGKVGDDEFGRMLADILRLNNVDNSGMRFDHNARTALAFVTLRADGEREFLFFRHPSADMLLSESELDKNLIQKAKIFHYGSISLIEEPCRSTQLAAMKIAKASGSLLSYDPNLRLPLWPSEDAARKEIMSIWNLADVIKISEDEITFLTGGDDPYDDNVVLQKLFHPDLKLLVVSEGPNGCRYYTQEFKGRVGGVKVKPVDTTGAGDAFVSGLLNSLASDLTLLKDEKKLREALLFANACGAITVTERGAIPAMPSMDAVQDLLTSSASHRS

SEQ ID NO: 90 - protein, 343 aa, Noccaea caerulescens
MGENAISGNLKNLTIDTGDSETLVVCFGEMLIDFVPTVGGVSLAEAPAFKKAPGGAPANVAVGVSRLGGSSAFIGKVGDDEFGRMLADILRLNNVDNSGMRFDHNARTALAFVTLRADGEREFLFFRHPSADMLLLESELDKNLIQKAKIFHYGSISLIEEPCRSTQLAAMKIAKASGSLLSYDPNLRLPLWSSEEAARKEIMSIWNLADVIKVSEDEITFLTGGDDPFDDDVVLQKLFHPSLKLLVVSEGPNGCRYYTQEFKGRVGGVKVKAVDTTGAGDAFVSGLLNSLASDLTLLTDEKKLREALLFANACGAITVTERGAIPAMPTMKAVQDLLSSARS

SEQ ID NO: 91 - protein, 343 aa, Brassica rapa
MGENAISGNLKNLKIDTGRSETLVVCFGEMLIDFVPTVGGVSLAEAPAFKKAPGGAPANVAVGVSRLGGSSAFVGKVGDDEFGRMLADILRLNNVDNSGMRFDHKARTALAFVTLRGDGEREFLFFRHPSADMRLLESELDKNLIQKAKIFHYGSIGLIEEPCRSTQLAAMKIAKASGSLLSYDPNLRLPLWPSEEAARKEIMSIWNLADVIKISEEEITFLTGGDDPYDDEVVLRKLFHPNLKLLVVSEGPNGCRYYTQEFKGRVGGVKVKAVDTTGAGDAFVSGLLNSLASDLTLLTDEKKLREALLFANACGAITATERGAIPAMPTMDAVQDLLSSSRS

SEQ ID NO: 92 - protein, 343 aa, Brassica oleracea
MGEKAISGNLKKLTIDTGRSETLVVCFGEMLIDFVPTVGGVSLAEAPAFKKAPGGAPANVAVGVSRLGGSSAFVGKVGDDEFGKMLAEILKLNNVDNSGMRFDHKARTALAFVTLRADGEREFLFFRHPSADMLLTESELDKNLIQKAKIFHYGSISLIKEPCRSTHLAAMKIAKAAGSLLSYDPNLRLPLWPSEEAARKEIMSIWELADVIKISEDEITFLTGGDDPYSDEVVLQKLFHPNLKLLVVSEGPNGCRYYTKEFKGRVGGVKVKAVDTTGAGDAFVSGLLNSLASDLTLLTDEKKLREALLFANACGAITVTERGAIPAMPTMDAVQELLKSSGS

SEQ ID NO: 93 - protein, 343 aa, Raphanus sativus
MCENAMSGHLKKLTIDTGRSETLVVCFGEMLIDFVPTVGGVSLAEAPAFKKAPGGAPANVAVGVSRLGGSSAFIGKVGDDEFGKMLAEILRLNNVDNSGMRFDHKARTALAFVTLRADGEREFLFFRHPSADMLLTESELDKNLIQKAKIFHYGSISLIKEPCRSTHLAAMKIAKAAGSLLSYDPNLRLPLWPSEEAARKEIMSIWHLADVIKISEDEITFLTGGDDPYSDEVVLQKLFHPNLKLLVVSEGPNGCRYYTKAFKGRVGGVKVKAVDTTGAGDAFVSGLLNSLASDLTLLTDEKKLREALLFANACGAITVTERGAIPAMPTMDAVQELVKSSRS

SEQ ID NO: 94 - protein, 356 aa, Brassica napus
MGENAISGSFETISVLLHISGDLKNIAIDTGRSETLVVCFGEMLIDFVPTVGGVSLAEAPAFKKAPGGAPANVAVGVSRLGGSSAFVGKVGDDEFGRMLAEILRLNNVDSSGMRFDHEARTALTFVTLRGDGEREFLFFRHPSADMRLLESELDKNLIQKAKIFHYGSISLIEEPCRSTQLAAMKIAKASGSLLSYDPNLRLALWPSEEAARKEIMSIWNLADVIKISEEEITFLTGGDDPYDDEVVLQKLFHPNLKLLVVSEGPNGCRYFTQEFKGKVGGVKVKAVDTTGAGDAFVSGLLNSLASDLTILNDEKKLREALLFANACGAITATERGAIPAMPTMNAVQDLLSSSRS

SEQ ID NO: 95 - protein, 346 aa, Juglans regia
MATNPTPGGSEDLPSDTQGTRDNNSLVVCFGEMLIDFVPTVGGVSLAEAPAFKKAPGGAPANVAVGISRLGGSSAFIGKVGDDEFGYMLADILKQNNVDNSGVRFDHNARTALSFVTLRADGEREFLFFRHPSADMLLHESELDIDLIKQARIFHYGSISLIDEPCRSTHLAAMSIARKSGCILSYDPNLRLPLWPSAEAARKGIMNIWDQADIIKISEEEITFLTGGDDPYDDNVVLKKLFHPNLKLLVVTEGSEGCRYYTKEFRGRVAGVKVKPVDTTGAGDAFVSGILNSLASDLNLIQDEKRLREALAFANACGALTVIERGAIPALPTKEAVLQFLPKVAA

SEQ ID NO: 96 - protein, 358 aa, Pyrus x bretschneideri
MANNLAPALDIKLVLREDSKDASSDKKGGSQDDKSLVVCFGEMLIDFVPTVGGVSLAEAPAFKKAPGGAPANVAVGIARLGGSAGFIGKVGDDEFGYMLADILKQNNVDNSGVRFDPSARTALAFVTLRADGEREFLFFRHPSADMLLHESELDINLIKRAKIFHYGSISLIDEPCRSTHIAAMKIAKKSGCILSYDPNLRLPLWPSEDAARKGIMSIWDQADIIKISEDEIRFLTGGDDPYDDNVVLTKLFHPNLRLLVVTEGSEGCRYYTQKFHGRVGGVKVKPVDTTGAGDAFVGGVLNSIASNLSLFQDEKGLREALLFANACGALTVTERGAIPAMPTREAVLQCLKQAADKK

SEQ ID NO: 97 - protein, 348 aa, Theobroma cacao
MAGNLTTGRAEDLSANTNGKSADNNSIVVCFGELLIDFVPTVGGVSLAEAPAFKKAPGGAPANVAVGVARLGGSSAFIGKVGDDEFGHMLADILRQNNVDISGMRFDHSARTALAFVTLRADGEREFLFFRHPSADMRLHESELDINLIKQAKIFHYGSISLIEEPCKSAHLAAMNIAKRSGSILSYDPNLRLPLWPSSEAAQTGIMSIWDQADLIKVSEDEIKFLTGGDDPYDDNVVMKKLYHPNLKLLVVTEGSEGCRYYTKAFKGRVPGIKVKPVDTTGAGDAFVSGLLSSLASDLKLFEDEERLREALNFANACGALTVTERGAIPAMPMKKAVIDALTKFAAS

SEQ ID NO: 98 - protein, 362 aa, Gossypium raimondii
MAGKLTTGNITGIEKLHIVGGAEDRTAITNGNAAKNKPLVVCFGELLIDFVPTVGGVSLAEAPAFKKAPGGAPANVAVGVSRLGGSSAFVGKVGDDEFGYMLADILKQNNVDNSGMRFDRTARTALAFVTLKADGEREFMFFRHPSADMRLHESELNTNLIKQANIFHYGSISLIEEPCKSAHLAAMNIAKKSGSMLSYDPNLRLPLWPSPEAARKGIMSIWDQSDIIKVSEDEITFLTGGDDPYDDNVVMKKLFHPNLKLLVVTEGSEGCRYYTKAFKGRVPGIKVKPVDTTGAGDAFVSGLLNSLASDSKLFQDEKRLRDALLFANVCGALTVTERGAIPSLPTKTAVLDALNKHSASKK

SEQ ID NO: 99 - protein, 371 aa, Vitis vinifera
MSKSNRSKDDEARMSGCCFPVNLDRSMKSGLSFKKSSSGSFKGESQEKRPLVVCFGEMLIDFVPTVGGVSLSESPAFKKAPGGAPANVAVGISRLGGSSAFIGKVGEDEFGFMLADILKQNNVDSSGMRFDRNARTALAFVTLRDDGEREFMFFRNPSADMLLRESELDANLIKKASIFHYGSISLIEEPCKSAHLAAMNIARKSGSILSYDPNLRLPLWPSPETARKTIMSIWNQADLIKISEEEITFLTGGDDPNDDNVVLKKLYHSNLKLLVVTEGSNGCRYYTKEFKGRVTGVKVKAVDTTGAGDAFVGGILNSLAADLNLYKNEEKLREALLFANACGAITVTERGAIPALPTKEAVLQILPKANA

SEQ ID NO: 100 - protein, 358 aa, Fragaria vesca
MASTPNSVLDDIKMLDLAKNSETPSADPEIKDNKSMVVCFGEMLIDFVPTVAGVSLAEAPAFKKAPGGAPANVAVGISRLGGSAAFIGKVGDDEFGKMLADILKQNNVNTSGVRFDHNARTALAFVTLRADGEREFLFFRNPSADMLLHESELEISLLQQAKIFHYGSISLIEEPCRSTHQAAMNIAKKSGCILSYDPNLRLALWPSAEFARTEIMSIWDQADIIKISEEEITFLTGGDDPYDDNVVLTKLFHPNLKLLIVTEGSEGCRYYTQNFRGRVAGIKVKAIDTTGAGDAFVGGILNSIASDSNLFQDEEGLREALLFANACGAITVTERGAIPAMPTTEAVLQFLAQNADKK

SEQ ID NO: 101 - protein, 347 aa, Nicotiana tabacum
MADESISGNLKDLSLNTNGAATKQSHLVVCFGEMLIDFVPTVGGVSLAEAAAFEKAPGGAPANVAVCIAKLGGSSAFIGKVGEDDFGHMLADILKQHNVDNSGMRFDHDARTALAFVTLTAEGEREFVFFRNPSADMLLRESELDVDLVKKATIFHYGSISLIEEPCRSTQLAAMDIAKRSGSILSYDPNLRLPLWPSEDAARSGIMSVWNLADVIKISEDEISFLTGADDPNDDEVVLKRLFHPNLKLLLVTEGSAGCRYYTKEFKGRVHGIKVKAVDTTGAGDAFVGGILKCLASDADLYQDEKRLRDALFFANACAALTVTGRGGIPSLPSQEAVQQTLTEVTA

SEQ ID NO: 102 - protein, 347 aa, Capsicum annuum
MADESISGNLKDLCLNRNGVASNNSHLVVCFGEMLIDFIPTVGGVSLAEAPAFEKAPGGAPANVAVCVSKLGGSSAFIGKVGDDEFGRMLADILKQHNVDNSGMRFDHDARTALAFVTLTAEGEREFVFFRNPSADMLLQESELDVNLIKKATIFHYGSISLIDEPCRSTHLAAMDIAKRSGSILSYDPNLRLPLWPSEDAARSGIMSVWNLADIIKISEDEISFLTGADDPNDDEVVLKRLFHPNLKLLLVTEGSAGCRYYTKEFKGRVSSIKVKAVDTTGAGDAFTGGILKCLASDTDLYQDEKRLREAIFFANVCAALTVTGRGGIPSLPTQDAVQRTLTEVAT

SEQ ID NO: 103 - protein, 429 aa, Glycine max
MVWYHTRAYNHSFKQNNTNLHAFINQTPTDFSLHSVHCVPSLLFLSSLYKVLLTPTKTHSCLFLLLPALHLQYSVFLVSIQSHTSIALMAHFTSSGKSDDLTKEDCKGQSALVVCFGEILIDFVPTVGGVSLAEAPAFKKAPGGAPANVAVGISRLGGSSAFVGKVGADEFGYMLADILKQNDVETSGMKFDPNARTALAFVTLRADGEREFLFFRNPSADMLLQESELDKNLIKKAKIFHYGSISLIDEPCKSAHLAAMRFAKESGCILSYDPNLRLALWPSAEAARDGIMSIWDQADVIKISEDEITFLTGGDDPYDDNVVLKKLFHPNLKLLIVTEGSEGCRYYTKEFKGRVAGVKVKPVDTTGAGDAFVSGIIYSLASDQSLFQNEEHLRKALHFANVCGAITVTERGAIPALPTKEAVLQFAAT

SEQ ID NO: 104 - protein, 345 aa, Vigna radiata
MAHHISSDHSNDLRKEDCKETGSLVVCFGEMLIDFVPTVGGVSLAEAPAFKKAPGGAPANVAVGISRLGGSSAFIGKVGADEFGYMLADILKQNNVETSGMRFDSNARTALAFVTLRADGEREFLFFRNPSADMLLQESELDKNLLKQARIFHYGSISLIDEPCKSAHLAAMRIAKNSGCILSYDPNLRLALWPSADAARKSIMDIWDQADVTKISEDEITFLTGGDDPYDDNVVLKKLFHPNLKLLIVTEGSQGCRYYTKAFKGRIAGVKVKPVDTTGAGDAFVSGILYSIASDQTIFQDEKRLRKALYFANICGALTVTGRGAIPALPTKEAILQFLLEEAVI

SEQ ID NO: 105 - protein, 351 aa, Citrus clementina
MADNHNPTSGAGSKDLSASMDGGSGAYDRLVVCFGEMLIDFVPTVGGVSLAEAPAFKKAPGGAPANVAVGISRLGGSSAFVGKLGDDEFGYMLANILKENNVDTSGVRYDSTARTALAFVTLRADGEREFLFFRHPSADMLLCESELDKNLIKQGSIFHYGSISLIAEPCRSTQLAAMNLAKESGSILSYDPNLRLPLWPSEEAAREGIMSIWDQADIIKVSDDEITFLTGGDDHNDDNVVLEKLFHPNLKLLIVTEGSKGCRYYTKEFKGRVPGVKTKAVDTTGAGDSFVSGILNCLAADQNLIKDENRLREALLFANACGALTVTERGAIPALPTKEAALKLLHTVAAL

SEQ ID NO: 106 - protein, 347 aa, Solanum lycopersicum; identical to SEQ ID NO: 85

SEQ ID NO: 107 - DNA, 1887 bp, Solanum lycopersicum
ggcacgagattgatcctcattctgcataagaaaattccggtacatttcatgccgtgtcggtgtcagcaaggtacaccaaaagtagtaaacagggtggcaaaaaagaggaggatttagcaatttgttgatttttccttctttctttctttcttgtcaacttcaaaaccccatcggctctctttcaaggttcccctttctttctctattaacgcctctctctgagttcttgttgatatttccaaagaccccatttttaacactgctcacaatatggctggcgaatccatttcaggcaatttaaaagacctttccttgaatagaaatggtgcggtgtcaaagaagtctcatttagttgtttgctttggggagatgctcattgacttcatcccaactgttgctggagtttcacttgcggaagctcctgcctttgaaaaagctcccggtggtgcacctgctaatgttgctgtgtgcatctcaaagttagggggttcatctgcttttattggaaaggttggtgacgatgagttcggccgtatgttggctgacattttgaagcaaaacaatgttgacaattccggcatgcggtttgatcatgatgcaaggactgcactggccttcattacactcacagctgaaggtgagcgggagttcgtgtttttccgtaatcctagtgctgatatgcttcttcgggagtcagaactcgatgtagatcttattaaaaaggccaccatcttccattatggttcaattagtttgatcgacgaaccttgtaggtcaacacaccttgctgcaatggacattgccaaaagatcaggtagcatactgtcgtatgatccaaacctgagattgcctttatggccttcagaagatgctgctcgaagtggaataatgagtgtatggaacctagcagatattattaagataagtgaggatgaaatttcattcttgactggagccgacgatccaaatgatgacgaggtggtgttgaagaggcttttccatcctaatctgaagcttttgcttgtaactgaaggttcagctggttgcagatattacaccaaggaattcaagggaagagtaaattcgatcaaggtaaaagctgttgatacaactggtgctggtgatgcatttactggtggagttctcaagtgtctagcttctgatgctagtctttatcaggatgaaaagcggttaagggaggctatcttttttgccaatgtttgtgctgccctgacagtgacaggaagaggtggaatcccttcccttcctacacaagatgcagttcgacaaactcttgccgaggtcactgcatgagaaggcagaacaaagttttgttctcttcacactgtatctgcattattctagatttattttcacaatgatcgatttattttgttttcgtctctggcatctgttggtcggttcctctctttggaaagaagttgcagccaacgagacatgcagggaaaaatagggtagcgcgtcttctgtcagtcatgcaaggaaatgctggaaagcctttttcgctaagtcaaaatacaagctgttatgtctccgttacatatctgatccttgttacggatccatcagaagccaagatagtgaaggttgttaacattggttattgagatttactgcgtgtagagagaagaacaaaaggtggacatgcatttaacgactatcagcttttgtttgttttaatatgtttccttttcaagaacctttctgtttttgtttcctttaaagtgtctgtattataaggtgacttcaatgctgtcttgattagaaatcagcagaacaaaaaatattacttatgcagttatgtggtttgatgtactactcagaaatcagaataatatgagtctcatactgttgatctcttccatc

SEQ ID NO: 108 - DNA, 21 bp, Artificial sequence
  Single strand DNA oligonucleotide
tcttaccaaaaggcaaaaccc

SEQ ID NO: 109 - DNA, 21 bp, Artificial sequence
  Single strand DNA oligonucleotide
aaattcaataacgcgattccc

SEQ ID NO: 110 - DNA, 21 bp, Artificial sequence
  Single strand DNA oligonucleotide
catgcgtttgcaaatagaagc

SEQ ID NO: 111 - DNA, 21 bp, Artificial sequence
  Single strand DNA oligonucleotide
accagctttcaagaaagctcc

SEQ ID NO: 112 - DNA, 21 bp, Artificial sequence
  Single strand DNA oligonucleotide
caggaagcttgatgaggagtg

SEQ ID NO: 113 - DNA, 21 bp, Artificial sequence
  Single strand DNA oligonucleotide
caagattcagttcatctggcc

SEQ ID NO: 114 - DNA, 21 bp, Artificial sequence
  Single strand DNA oligonucleotide
tcatacatgtcaaagctagcc

SEQ ID NO: 115 - DNA, 21 bp, Artificial sequence
  Single strand DNA oligonucleotide
gcactagggttccggtagaac

SEQ ID NO: 116 - DNA, 21 bp, Artificial sequence
  Single strand DNA oligonucleotide
aacaacatatcagcactcggg

SEQ ID NO: 117 - DNA, 21 bp, Artificial sequence
  Single strand DNA oligonucleotide
ttgaatgaggtgatcgatgtg

SEQ ID NO: 118 - DNA, 22 bp, Artificial sequence
  Single strand DNA oligonucleotide
gcgtggaccgcttgctgcaact

SEQ ID NO: 119 - DNA, 21 bp, Artificial sequence
  Single strand DNA oligonucleotide
ttcttctccttgtccttcctt

SEQ ID NO: 120 - DNA, 20 bp, Artificial sequence
  Single strand DNA oligonucleotide
cacttccatggccttcaaat

SEQ ID NO: 121 - DNA, 20 bp, Artificial sequence
  Single strand DNA oligonucleotide
cgctctctcagcttcctctc

SEQ ID NO: 122 - DNA, 23 bp, Artificial sequence
  Single strand DNA oligonucleotide
ggagattcttgtgttgaaccatc

SEQ ID NO: 123 - DNA, 19 bp, Artificial sequence
  Single strand DNA oligonucleotide
acttggtggctcctctgct

SEQ ID NO: 124 - DNA, 20 bp, Artificial sequence
  Single strand DNA oligonucleotide
ggcaaggttcctcaatcaaa

SEQ ID NO: 125 - DNA, 20 bp, Artificial sequence
  Single strand DNA oligonucleotide
gaaagaacggtgtcgacgat

SEQ ID NO: 126 - DNA, 21 bp, Artificial sequence
  Single strand DNA oligonucleotide
ttaacaaggagaagccgtaca

SEQ ID NO: 127 - DNA, 26 bp, Artificial sequence
  Single strand DNA oligonucleotide
attatatccaaaacataatctctctc

SEQ ID NO: 128 - DNA, 20 bp, Artificial sequence
  Single strand DNA oligonucleotide
aatctgcttctgagcctcct

SEQ ID NO: 129 - DNA, 20 bp, Artificial sequence
  Single strand DNA oligonucleotide
gcgggaagcaagagcgtgtt

SEQ ID NO: 130 - DNA, 20 bp, Artificial sequence
  Single strand DNA oligonucleotide
ctcctcgggttgctatgatg

SEQ ID NO: 131 - DNA, 21 bp, Artificial sequence
  Single strand DNA oligonucleotide
tagaaatggtgcggtgtcaaa

SEQ ID NO: 132 - DNA, 20 bp, Artificial sequence
  Single strand DNA oligonucleotide
aacatacggccgaactcatc

(19) United States
(12) Patent Application Publication
(10) Pub. No.: US 2018/0148732 A1
(43) Pub. Date: May 31, 2018

(54) METHODS OF MODIFYING OIL CONTENT IN PLANTS AND PLANTS PRODUCED THEREBY

(71) Applicant: The State of Israel, Ministry of Agriculture & Rural Development, Agricultural Research Organizat, Rishon-Lezion (IL)

(72) Inventors: David GRANOT, Jerusalem (IL); Ofer Stein, Rehovot (IL)

(21) Appl. No.: 15/823,658

(22) Filed: Nov. 28, 2017

Related U.S. Application Data
(60) Provisional application No. 62/427,301, filed on Nov. 29, 2016.

(51) Int. Cl. C12N 15/82; C12N 9/12

(52) U.S. Cl. CPC C12N 15/8247; C12N 9/1205; C12N 15/8267; C12N 15/8234; C12Y 207/01004

(57) ABSTRACT

A method of modifying oil content of a plant is provided. The method comprising: (a) modulating activity or expression of a fructokinase (FRK) in seeds of a plurality of plants; and (b) selecting a seeds of the plurality of plants or progeny thereof exhibiting the modified oil content as compared to the oil content of seeds of the same species not subjected to step (a).

### RELATED APPLICATIONS

This application claims the benefit of priority under 35 USC § 119(e) of U.S. Provisional Patent Application No. 62/427,301 filed on Nov. 29, 2016, the contents of which are incorporated herein by reference in their entirety.

### SEQUENCE LISTING STATEMENT

The ASCII file, entitled 71857SequenceListing.txt, created on Nov. 28, 2017, comprising 329,716 bytes, submitted concurrently with the filing of this application is incorporated herein by reference.

### FIELD AND BACKGROUND OF THE INVENTION

The present invention, in some embodiments thereof, relates to methods of modifying oil content in plants and plants produced thereby.

Sucrose is an important end product of photosynthesis and the primary carbon source for metabolism in sink tissues of many plants, including Arabidopsis. Sucrose must be cleaved by either sucrose synthase (SUS) into UDP-glucose and fructose, or by invertase into glucose and fructose, before it can be further metabolized (Dennis & Blakeley, 2000). The free hexoses, fructose and glucose, must then be phosphorylated by fructokinase (FRK) or hexokinase (HXK) before they can enter metabolic pathways. FRK and HXK are distinguished by their substrate specificities and affinities (Renz & Stitt, 1993; Dai et al., 2002; Granot, 2007). FRK phosphorylates only fructose; whereas HXK phosphorylates both glucose and fructose. However, the affinity of FRK for fructose is two orders of magnitude higher than that of HXK for fructose. It is, therefore, likely that fructose is phosphorylated primarily by FRK (Granot, 2007).

Several FRKs have been identified in a number of plant species including tomato (Solanum lycopersicum), potato (Solanum tuberosum), maize (Zea maize), soybean (Glycine max), barley (Hordeum vulgare), spinach (Spinacia oleracea) and pea (Pisum sativum) (Pego & Smeekens, 2000). In tomato, for example, currently the best characterized plant species with regard to FRK, four FRK genes have been identified (SlFRK1-4) encoding enzymes with different intracellular localization and biochemical characteristics. Three of the tomato FRK enzymes are located in the cytosol and a single tomato FRK (SlFRK3) is found in plastids (Kanayama et al., 1997; Kanayama et al., 1998; German et al., 2002; German et al., 2004; Damari-Weissler et al., 2006; Granot, 2007). FRKs are expressed at different levels in almost all plant tissues, yet, in tomato, SlFRK4 is expressed specifically in stamens and pollen (German et al., 2002; German et al., 2003; German et al., 2004; David-Schwartz et al., 2013; Granot et al., 2013).

Increasing evidence suggest that FRKs are important for vascular development. The tomato SlFRK2 is essential for proper xylem development, and the xylem vessels in stems of SlFRK2-antisense plants have thinner xylem secondary cell walls and those cells are narrower and deformed (Damari-Weissler et al., 2009). As a result, water conductance is reduced, causing severe growth inhibition and the wilting of young leaves (Damari-Weissler et al., 2009). The tomato plastidic FRK is also important for xylem development, as indicated by the fact that RNAi suppression of SlFRK3 decreases plant hydraulic conductivity and transpiration. Suppression of both the cytosolic SlFRK2 and the plastidic SlFRK3 yielded deformed xylem vessels and fibers with thin cell walls, implying that both genes play a role in xylem fiber development (Stein et al., 2016). FRK is also important for xylem fiber development in aspenwood (Populus tremula x tremuloides), in which the suppression of the cytosolic FRK2 yielded narrower xylem fibers perhaps due to a decrease in cellulose content (Roach et al., 2012).

Additional background art includes:

US 20150322450

### SUMMARY OF THE INVENTION

According to an aspect of some embodiments of the present invention there is provided a method of modifying oil content of a plant, the method comprising:

(a) modulating activity or expression of a fructokinase (FRK) in seeds of a plurality of plants; and

(b) selecting a seeds of the plurality of plants or progeny thereof exhibiting the modified oil content as compared to the oil content of seeds of the same species not subjected to step (a).

According to an aspect of some embodiments of the present invention there is provided a method of modifying germination rate of a plant, the method comprising:

(a) modulating activity or expression of a fructokinase (FRK) in seeds of a plurality of plants; and

(b) selecting a seeds of the plurality of plants or progeny thereof exhibiting modified germination rate as compared to the oil content of seeds of the same species not subjected to step (a).

According to an aspect of some embodiments of the present invention there is provided a method of modifying oil content of an oil seed plant, the method comprising modulating activity or expression of a fructokinase (FRK) in seeds of the oil seed plant.

According to an aspect of some embodiments of the present invention there is provided a method of modifying oil content of a plant, the method comprising modulating activity or expression of at least two fructokinases (FRKs) in seeds of the plant, thereby modifying the oil content of the plant.

According to some embodiments of the invention, the FRK comprises at least two FRKs.

According to some embodiments of the invention, the modifying and modulating is increasing.

According to some embodiments of the invention, the modifying and modulating is decreasing.

According to some embodiments of the invention, the increasing comprises over-expressing a heterologous nucleic acid sequence encoding FRK.

According to some embodiments of the invention, the nucleic acid sequence encoding the FRK is under a constitutively active promoter.

According to some embodiments of the invention, the nucleic acid sequence encoding the FRK is under a developmentally regulated promoter active specifically in the embryo.

According to some embodiments of the invention, the increasing comprises editing an endogenous nucleic acid sequence encoding the FRK.

According to some embodiments of the invention, the FRK or the at least two FRKs comprise an embryonic expressed FRK or homolog thereof.

According to some embodiments of the invention, the FRK or the at least two FRKs comprises a cytosolic FRK.

According to some embodiments of the invention, the FRK or the at least two FRKs comprise comprises a plastid FRK.

According to some embodiments of the invention, the at least two FRKs comprise AtFRK-6 and AtFRK-7 or a functional homolog thereof.

According to an aspect of some embodiments of the present invention there is provided an oil seed plant having been genetically modified or selected to modulate FRK activity in seeds thereof.

According to an aspect of some embodiments of the present invention there is provided an oil seed plant obtainable according to the method as described herein.

According to an aspect of some embodiments of the present invention there is provided a seed of the plant as described herein.

According to an aspect of some embodiments of the present invention there is provided oil of the plant as described herein.

According to some embodiments of the invention, the oil has increased TCA cycle metabolites and/or larger oil bodies as compared to oil of a plant of the same species not subjected to genetic FRK modulation.

According to an aspect of some embodiments of the present invention there is provided a method of producing oil, the method comprising:

providing seeds as described herein; and

extracting oil from the seeds.

According to an aspect of some embodiments of the present invention there is provided a nucleic acid construct comprising a nucleic acid agent encoding an FRK under a developmentally regulated promoter specifically active in the embryo.

According to an aspect of some embodiments of the present invention there is provided a plant of a plant cell comprising the nucleic acid construct.

According to an aspect of some embodiments of the present invention there is provided a seed of a plant cell comprising the nucleic acid construct.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawings will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 shows the phylogenetic relationships between tomato and Arabidopsis fructokinases. Phylogenetic evolutionary analysis was conducted using MEGA version 4 (Tamura et al., 2007). This phylogenetic tree is based on the following protein sequences: SlFRK1 (NP_001233893), SlFRK2 (NP_001233888), SlFRK3 (NP_001234396), SlFRK4 (NP_001234206), At5g51830 (NP_199996), At1g66430 (NP_564875), At4g10260 (NP_192764), At3g59480 (NP_191507), At1g06030 (NP_172093), At1g06020 (NP_172092) and At2g31390 (NP_180697).

FIGS. 2A and 2B show the predicted subcellular localization of AtFRKs. Full-length AtFRK protein sequences were used to predict their localization. (FIG. 2A) Best subcellular localization prediction by WoLF PSORT and (FIG. 2B) subcellular localization prediction by TargetP 1.1. cTP—chloroplast transit peptide, mTP—mitochondrial transit peptide, SP—secretory pathway. Localization: C—chloroplast,—other.

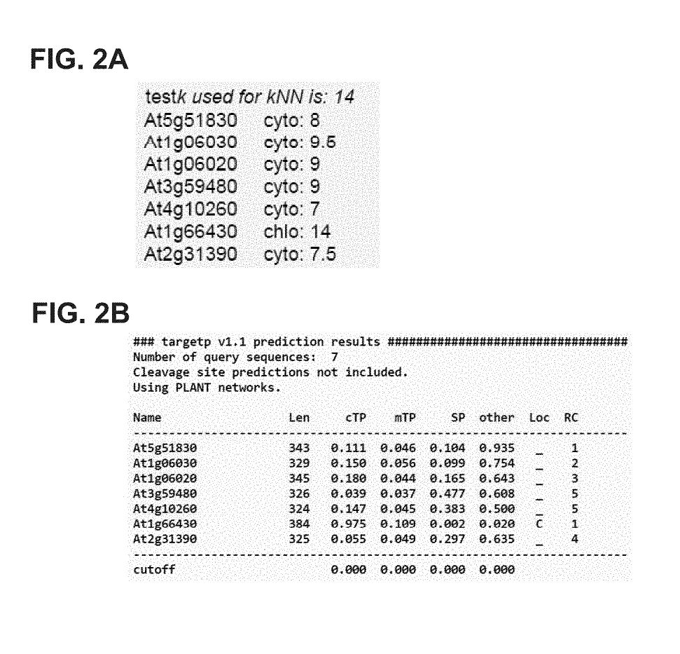

FIGS. 3A, 3B and 3C show the identification of the AtFRK T-DNA lines. (FIG. 3A) Exon-intron structure and positions at which T-DNA was inserted into the AtFRK genes. Exons are depicted as black boxes, introns are the lines between the boxes and the 5- and 3-UTRs are shown in gray. Scale bar=200 bp. (FIG. 3B) PCR analysis of genomic DNA to determine the homozygosity of the T-DNA insertion lines through the absence of the WT allele. Amplification was performed with right and left primers of each of the lines and AtHXK1 primers were used as a positive control. The confirmation of the T-DNA insertion in each of the lines was performed with the RP or LP for each gene and the left border primer LBb1 (not shown). Primers are listed in Table 3. (FIG. 3C) PCR analysis of cDNA to determine the presence or absence of the WT allele mRNA in the T-DNA insertion lines. AtHXK1 was used as a control. RNA from frk1 and the double-mutant frk6, frk7 was extracted from leaves. RNA for frk4 was extracted the top of the inflorescence, including the shoot apical meristem and developing flowers. Primers used for the amplification of the cDNA are listed in Table 3.

FIGS. 4A, 4B, 4C, 4D and 4E show seed phenotypes. Seeds were harvested, placed on black paper and photographed under a binocular microscope at maximal magnification. (FIG. 4A) WT seeds, (FIG. 4B) frk7 seeds, (FIG. 4C) frk6 seeds, (FIG. 4D) frk6 frk7 double-mutant seeds and (FIG. 4E) T3 homozygous seeds of a cross between frk6 frk7 and a line over-expressing SIFRK1. Bars—500 μm.

FIGS. 5A, 5B, 5C and 5D are scanning electron microscope micrographs of the different seeds. (FIG. 5A) WT seed, (FIG. 5B) frk6 frk7 seed, (FIG. 5C) WT seed surface and (FIG. 5D) frk6 frk7 seed surface. Scale bars: (FIGS. 5A-B)—100 μm; FIGS. 5C-D—20 μm.

FIGS. 6A, 6B, 6C, 6D, 6E, 6F and 6G are scanning electron microscope micrographs of Arabidopsis seeds. WT and frk6 frk7 double-mutant seeds were photographed using SEM. (FIG. 6A) WT seed. (FIGS. 6B-G) frk6 frk7 seeds wrinkled at various degrees. Bars—100 μm.

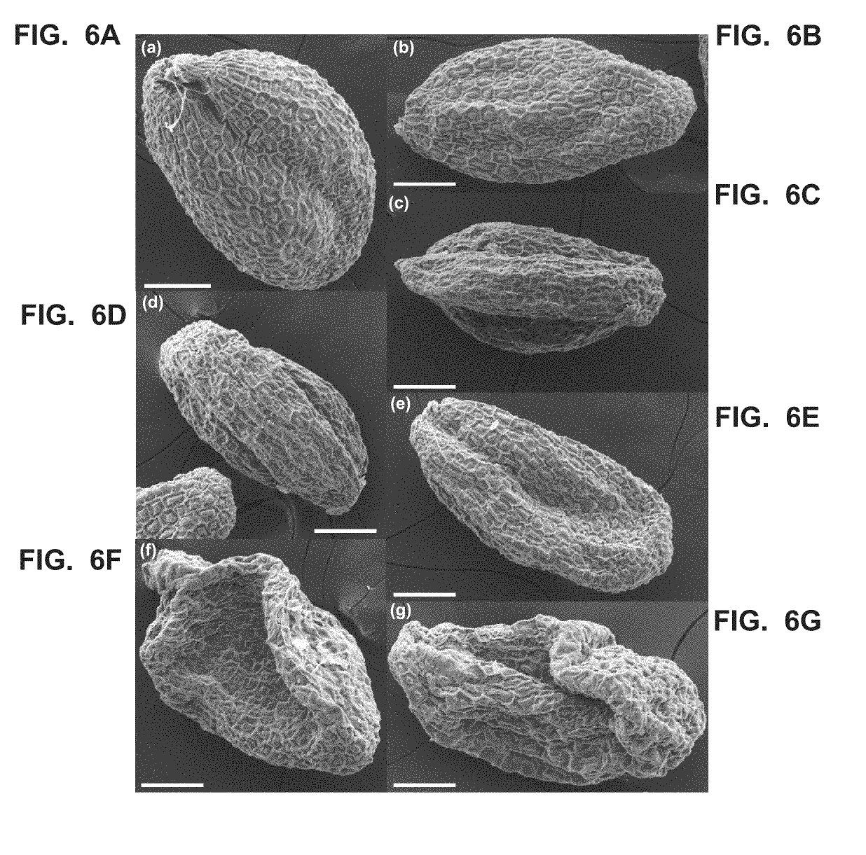

FIGS. 7A, 7B, 7C, 7D, 7E, 7F, 7G, 7H, 7I and 7J show AtFRK double-mutant growth in the presence of different sugars. Seeds were sterilized and sown on ½ strength MS media supplemented with different sugars. (FIGS. 7A, 7C, 7E, 7G) WT; (FIGS. 7B, 7D, 7F, 7H) frk6 frk7 mutant. (FIGS. 7A-B) no sugar added, (FIGS. 7C-D) 1% sucrose, (FIGS. 7E-F) 1% glucose, (FIGS. 7G-H) 1% fructose and (FIGS. 7A-J) 1% mannitol. Plates were photographed under a binocular microscope 10 days after they were transferred to the growth chamber. Scale bars—5 mm.

FIG. 8 shows that the frk6 frk7 double-mutant germinated relatively slowly. Seeds were harvested from WT, frk6, frk7 and the double-mutant plants grown under even-day conditions (12-h photoperiod) at the same time. Sixty to ninety seeds from each plant, from five individual plants per line, were surface-sterilized and sown on half-strength MS medium without any additional sugar. Following three days of stratification at 4° C., plates were transferred to a growth chamber and the germination rate was monitored by visualization of clear radicle emergence using a magnifying glass every 12 h for 4 days and after 1 week. Bars indicate standard deviation (n=5). An asterisk indicates a statistical significant difference (p<0.05).

FIG. 9 shows that only the frk6 frk7 double-mutant had a slower germination rate. The different double-mutant combinations were surface-sterilized and sown on half-strength MS medium without any additional sugar. Following three days of stratification at 4° C., plates were transferred to a growth chamber. Plates were photographed after one week.

FIGS. 10A, 10B and 10C show that SlFRK1 over-expression complements the frk6 frk7 growth arrest. Seeds were sterilized and sown on half-strength MS media with no sugar added. Plates were photographed under a binocular microscope 10 days after they were transferred to the growth chamber (FIG. 10A) WT, (FIG. 10B) frk6 frk7 and (FIG. 10C) homozygous frk6 frk7 over-expressing SIFRK1. Scale bars—5 mm.

FIG. 11 is an analysis of the expression of AtFRKs during seed development as depicted by the BAR eFP browser (Winter et al., 2007) based on the dataset generated by (Schmid et al., 2005) to create a gene-expression map for Arabidopsis development.

FIG. 12 is an analysis of the expression of AtFRKs during seed development as depicted by the BAR eFP browser (Winter et al., 2007) based on the dataset generated by (Le et al., 2010) of gene expression profiling from laser-captures of micro-dissected seeds over the course of seed development.

FIGS. 13A and 13B show that frk6 frk7 double-mutant exhibits altered primary metabolism. (FIG. 13A) PCA of primary metabolite levels. (FIG. 13B) Log2 values of the relative metabolic contents are presented as a heat map. Metabolic content was analyzed using GC-MS (n=6). Significant differences between the mutant and the wild type (as determined using Student's t-test) are denoted by one asterisk (P<0.05) or two asterisks (P<0.01). Detailed results of the assay are presented in Supplemental File 1.

FIGS. 14A and 14B show that frk6 frk7 double-mutant exhibits an altered lipid profile. Twenty dry seeds per sample were used for lipid extraction. Lipid profiles were analyzed using GC-MS (n=6). (FIG. 14A) PCA of the lipid profile. (FIG. 14B) Log2 values of the relative lipids are presented as a heat map. Significant differences between the mutant and the wild type (as determined using Student's t-test) are denoted by one asterisk (P<0.05) or two asterisks (P<0.01).

FIGS. 15A, 15B, 15C and 15D are transmission electron micrographs of mature seed embryonic cells. (FIGS. 15A, 15C) WT. (FIGS. 15B, 15D) frk6 frk7 mutant. OB—oil bodies, PB—protein bodies. Bars: (FIGS. 15A, 15B) 10 μm; (FIGS. 15C, 15D) 5 μm.

FIGS. 16A, 16B, 16C and 16D show that the seeds of the quadruple and penta mutants have more pronounced phenotype. Seeds were placed on black paper and photographed under a binocular microscope at maximal magnification. (FIG. 16A) Col-0 seeds, (FIG. 16B) frk6 frk7 double-mutant seeds, (FIG. 16C) frkl frk4 frk6 frk7 quadruple-mutant seeds and (FIG. 16D) frk1 frk3 frk4 frk6 frk7 penta-mutant seeds. Bars—500 μm.

FIG. 17 shows that AtFRK quadruple- and penta-mutants exhibit leaf wilting after bolting. WT, quadruple-mutant and penta-mutant Arabidopsis plants were grown in a growth chamber for 8 weeks under even-day conditions (12 h of light each day) and photographed when their leaves started to wilt.

FIG. 18 is a simplified scheme of the suggested model for cytosolic and plastidic FRK for glycolysis and fatty acid synthesis in Arabidopsis. This scheme is adapted from (Baud et al., 2008) in which arrow thickness are proportional to net carbon fluxes based on biochemical data and transcriptional profiling of maturing seeds (White et al., 2000; Schwender et al., 2004; Schwender et al., 2006). ADP-Glc, adenosine diphosphoglucose; AcCoA, acetyl-coenzyme A; 1,3-BPG, 1,3-bisphosphoglycerate; DHAP, dihydroxyacetone-3-phosphate; E-4-P, erythrose-4-phosphate; Fm, fructose; Fm-1,6-P, fructose-1,6-bisphosphate; Fm-6-P, fructose-6-phosphate; GAP, glyceraldehyde-3 -pho sphate ; Glc, glucose; Glc-1-P, glucose-1-phosphate; Glc-6-P, glucose-6-phosphate; KG, alpha-ketoglutarate; OAA, oxaloacetate; 6-PG, 6-phosphogluconate; 6-PGL, 6-phosphogluconolactone; PEP, phosphoenolpyruv ate; 2-PGA, 2-pho sphoglycerate; 3-PGA, 3 -phosphoglycerate; R-5-P, ribose-5-phosphate; Ru-1,5-P, ribulose-1,5-bisphosphate; Ru-5-P, ribulose-5-phosphate; S-7-P, sedoheptulose-7-phosphate; UDP-Glc, uridine diphosphoglucose; Xu-5-P, xylulose-5-phosphate.

### DESCRIPTION OF SPECIFIC EMBODIMENTS OF THE INVENTION

The present invention, in some embodiments thereof, relates to methods of modifying oil content in plants and plants produced thereby.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Fructokinases (FRKs) catalyze fructose phosphorylation. The enzyme has previously been associated with a number of traits including secondary cell wall deposit.

Whilst reducing the present invention to practice, the present inventors have uncovered that FRKs are involved in seed storage accumulation which controls various processes within the plant including germination.

Specifically, as is illustrated hereinbelow and in the Examples section which follows, T-DNA knockout mutants for five of the seven FRKs were identified and used to study the role of FRKs in Arabidopsis.

Single knockouts of the FRK mutants did not exhibit any unusual phenotype. Double-mutants of AtFRK6 (plastidic) and AtFRK7 showed normal growth in soil, but yielded dark, distorted seeds. The seed distortion could be complemented by expression of tomato SIFRK1.

Seeds of the double-mutant germinated, but failed to establish on ½ MS plates. Seedling establishment was made possible by the addition of glucose or sucrose, indicating reduced seed storage reserves. Metabolic profiling of the double-mutant seeds revealed decreased TCA cycle metabolites and reduced fatty acid metabolism. Examination of the mutant embryo cells revealed smaller oil bodies, the primary storage reserve in Arabidopsis seeds.

Quadruple and penta FRK mutants showed growth inhibition and leaf wilting. Anatomical analysis revealed smaller trachea elements and smaller xylem area, accompanied by necrosis around the cambium and the phloem.

These results demonstrate overlapping and complementary roles of the plastidic and the cytosolic FRKs in seed storage accumulation and their potential use in modifying seed storage reserves.

Aside from modifying (e.g., increasing seed oil content), the present teachings increase seed longevity and allow seeds to maintain high germination percentage for a long period, Increasing shelf life is also desirable for edible seeds. In addition increasing oil content may improve resistance to biotic and abiotic stresses.

Thus, according to an aspect of the invention there is provided a method of modifying seed storage oil content of a plant, the method comprising:

(a) modulating activity or expression of a fructokinase (FRK) in seeds of a plurality of plants; and

(b) selecting seeds of said plurality of plants or progeny thereof exhibiting said modified oil content as compared to said oil content of seeds of the same species not subjected to step (a).

According to an alternative or an additional aspect there is provided a method of modifying germination rate of a plant, the method comprising:

(a) modulating activity or expression of a fructokinase (FRK) in seeds of a plurality of plants; and

(b) selecting a seeds of said plurality of plants or progeny thereof exhibiting modified germination rate as compared to said oil content of seeds of the same species not subjected to step (a).

According to an alternative or an additional aspect there is provided a method of modifying oil content of an oil seed plant, the method comprising modulating activity or expression of a fructokinase (FRK) in seeds of the oil seed plant.

According to an alternative or an additional aspect there is provided a method of modifying oil content of a plant, the method comprising modulating activity or expression of at least two fructokinases (FRKs) in seeds of the plant, thereby modifying the oil content of the plant.

As used herein “seed storage oil content” refers to the endosperm oil content of oil seed plant.

According to some embodiments, oil content refers to the amount (w, v) of oil per seed (e.g., 1000 seed) or the composition of the oil. In the latter case, the present inventors have found that modulation of FRK activity/expression directly correlates with metabolic changes (see Example 5).

As used herein “germination rate” refers to the rate by which a seed embryo develops into a seedling. As the embryo content (oil) affects germination, the present inventors have realized that this finding can also be harnessed towards controlling germination rate (see e.g., Example 3).

As used herein “modifying” refers to, in the context of oil content, changing the amount or composition of oil (e.g., per seed/1000 seed). In the context of germination rate, increasing or decreasing germination rate.

As used herein “modulating” refers to upregulation or downregulation of expression or activity of FRK.

As used herein “increasing” refers to a statistically significant increase.

According to one embodiment, the increase is by about 5%, about 10%, about 20%, about 30%, about 40%, about 50%, about 60%, about 70%, about 80%, about 90%, about 100% or more as compared to that of a plant of the same species and under the same growth conditions and developmental stage in which the activity or expression of FRK has not been modulated according to the present teachings, this plant (or organ, or tissue or cell thereof) is also referred to as “control”.

As used herein “decreasing” refers to a statistically significant decrease. According to one embodiment, the decrease is by about 5%, about 10%, about 20%, about 30%, about 40%, about 50%, about 60%, about 70%, about 80%, about 90%, about 100% as compared to control.

As used herein “fructokinase” or “FRK” refers to an enzyme of EC 2.7.1.4 that catalyzes the reaction ATP+D-fructose=ADP+D-fructose 6-phosphate in plants.

Plant FRKs may classified according to their cellular localization.

According to a specific embodiment, the FRK is a cytoplasmic FRK.

According to a specific embodiment, the FRK is a plastid FRK.

According to a specific embodiment the FRK is a plant FRK.

In order to increase the effect of modulating the activity or expression of FRK, the present teachings further contemplate modulating at least two FRKs.

Accordingly, for instance, the at least two FRKs comprise two plastid FRKs.

According to another embodiment, the at least two FRKs comprise two cytoplasmic (cytosolic) FRKs.

According to another embodiment, the at least two FRKs comprise one cytoplasmic FRK and one plastid FRK.

Examples of FRKs that can be used in accordance with the present teachings e.g., 1, 2, 3, 4, 5, 6 FRKs are provided hereinbelow.

According to a specific embodiment, the FRK comprises AtFRK-6 or a functional homolog of same and AtFRK-7 or a homolog of same.

According to a specific embodiment, the FRK is native to the plant in which the activity of the FRK is being modulated (also referred to as endogenous).

According to a specific embodiment, the FRK is ectopic (heterologous) to the plant (especially relevant for over expression). These can be homologous wild type sequences (e.g., from other plant species) or homologous synthetic sequences (e.g., designed by codon optimization or conservative substitutions, the latter altering the amino acid composition of the WT FRK but do not hamper the activity of the FRK).

As used herein “activity of FRK” refers to the phosphorylation activity of the enzyme but also to downstream effect thereof in modifying the content/composition of the oil in the endosperm.

Hence, according to the present teachings there is provided wild-type FRKs as well as homologous sequences thereof.

According to some embodiments of the invention, the FRK has an amino acid sequence (or nucleic acid sequence encoding said FRK) at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more say 100% homologous (e.g., identical) to the amino acid sequence of Wild Type (WT) FRK (see e.g., Table 1 below).

According to a specific embodiment, homologous sequences include both orthologous and paralogous sequences. The term “paralogous” relates to gene-duplications within the genome of a species leading to paralogous genes. The term “orthologous” relates to homologous genes in different organisms due to ancestral relationship. Thus, orthologs are evolutionary counterparts derived from a single ancestral gene in the last common ancestor of given two species (Koonin EV and Galperin MY (Sequence-Evolution-Function: Computational Approaches in Comparative Genomics. Boston: Kluwer Academic; 2003. Chapter 2, Evolutionary Concept in Genetics and Genomics. Available from: ncbi (dot) nlm (dot) nih (dot) gov/books/NBK20255) and therefore have great likelihood of having the same function.

One option to identify orthologues in monocot plant species is by performing a reciprocal blast search. This may be done by a first blast involving blasting the sequence-of-interest against any sequence database, such as the publicly available NCBI database which may be found at: ncbi (dot) nlm (dot) nih (dot) gov. If orthologues in rice were sought, the sequence-of-interest would be blasted against, for example, the 28,469 full-length cDNA clones from Oryza sativa Nipponbare available at NCBI. The blast results may be filtered. The full-length sequences of either the filtered results or the non-filtered results are then blasted back (second blast) against the sequences of the organism from which the sequence-of-interest is derived. The results of the first and second blasts are then compared. An orthologue is identified when the sequence resulting in the highest score (best hit) in the first blast identifies in the second blast the query sequence (the original sequence-of-interest) as the best hit. Using the same rational a paralogue (homolog to a gene in the same organism) is found. In case of large sequence families, the ClustalW program may be used [ebi (dot) ac (dot) uk/Tools/clustalw2/index (dot) html], followed by a neighbor-joining tree (wikipedia (dot) org/wiki/Neighbor-joining) which helps visualizing the clustering.

Homology (e.g., percent homology, sequence identity+sequence similarity) can be determined using any homology comparison software computing a pairwise sequence alignment.

As used herein, “sequence identity” or “identity” in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”.

Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. [Amino acid substitution matrices from protein blocks. Proc. Natl. Acad. Sci. U.S.A. 1992, 89(22): 10915-9].

Identity (e.g., percent homology) can be determined using any homology comparison software, including for example, the BlastN software of the National Center of Biotechnology Information (NCBI) such as by using default parameters.

According to some embodiments of the invention, the identity is a global identity, i.e., an identity over the entire amino acid or nucleic acid sequences of the invention and not over portions thereof.

According to some embodiments of the invention, the term “homology” or “homologous” refers to identity of two or more nucleic acid sequences; or identity of two or more amino acid sequences; or the identity of an amino acid sequence to one or more nucleic acid sequence.

According to some embodiments of the invention, the homology is a global homology, i.e., a homology over the entire amino acid or nucleic acid sequences of the invention and not over portions thereof.

The degree of homology or identity between two or more sequences can be determined using various known sequence comparison tools. Following is a non-limiting description of such tools which can be used along with some embodiments of the invention.

Pairwise global alignment was defined by S. B. Needleman and C. D. Wunsch, “A general method applicable to the search of similarities in the amino acid sequence of two proteins” Journal of Molecular Biology, 1970, pages 443-53, volume 48).

For example, when starting from a polypeptide sequence and comparing to other polypeptide sequences, the EMBOSS-6.0.1 Needleman-Wunsch algorithm (available from emboss(dot)sourceforge(dot)net/apps/cvs/emboss/apps/needle(dot)html) can be used to find the optimum alignment (including gaps) of two sequences along their entire length—a “Global alignment”. Default parameters for Needleman-Wunsch algorithm (EMBOSS-6.0.1) include: gapopen=10; gapextend=0.5; datafile=EBLOSUM62; brief=YES.

According to some embodiments of the invention, the parameters used with the EMBOSS-6.0.1 tool (for protein-protein comparison) include: gapopen=8; gapextend=2; datafile=EBLOSUM62; brief=YES.

According to some embodiments of the invention, the threshold used to determine homology using the EMBOSS-6.0.1 Needleman-Wunsch algorithm is 80%, 81%, 82%, 83%, 84%, 85%, 86%, 87%, 88%, 89%, 90%, 91%, 92%, 93%, 94%, 95%, 96%, 97%, 98%, 99%, or 100%.

TABLE 1
Arabidopsis FRKs homolog genes

| | | | Degree of |
|---|---|---|---|
| | | | homology |
| | | | (% to the |
| Arbidopsis thaliana | | Cellular | amino acid |
| FRK/SEQ ID NO: | Homologs/SEQ ID NO: 1-105 | Localization | sequence) |
| AtFRK1 | SIFRK2 (solanum lycopersicum) NP_001233888 | Cytosol | 83% |
| At2g31390 | FRK1 (camelina sativa) XP_010510201 | | 97% |
| AAM14251 | BnaA05g11350D (brassica napus) CDY10801 | | 95% |
| | FRK1 (populous euphratica) XP_011026435 | | 84% |
| | FRK2 (capsicum annum) XP_016578084 | | 84% |
| | FRK1 (Noccaea caerulescens) JAU82139 | | 94% |
| | FRK1 (brassica rapa) XP_009144211 | | 95% |
| | FRK1 (brassica oleracea) XP_013636348 | | 95% |
| | FRK1 (raphanus sativus) XP_018439834 | | 95% |
| | FRK1 (eucaliptus grandis) XP_010045714 | | 85% |
| AtFRK2 | SIFRK2 (solanum lycopersicum) NP_001233888 | Cytosol | 82% |
| At1g06030 | FRK2 (camelina sativa) XP_010475326 | | 95% |
| AAM13911 | FRK2 (brassica napus) XP_013665680 | | 90% |
| | FRK2 (glycine max) XP_003537935 | | 84% |
| | hypothetical protein (capsella rubella) XP_006304792 | | 93% |
| | FRK2 (Noccaea caerulescens) JAU50741 | | 90% |
| | FRK2 (brassica rapa) XP_009119097 | | 90% |
| | FRK2 (brassica oleracea) XP_013586994 | | 90% |
| | FRK2 (raphanus sativus) XP_018486754 | | 90% |
| | Fructokinase (nicotiana tabacum) AIE16180 | | 84% |
| | FRK2 (ricinus communis) XP_002533363 | | 83% |
| | FRK2 (cicer arietnum) XP_004507340 | | 84% |
| | hypothetical protein (citrus clementina) XP_006443006 | | 84% |
| | FRK2-like (vigna radiata) XP_014494566 | | 82% |
| | FRK2 (juglans regia) XP_018839987 | | 84% |
| AtFRK3 | SIFRK2 (solanum lycopersicum) NP_001233888 | Cytosol | 84% |
| At1g06020 | FRK3 (camelina sativa) XP_010501336 | | 96% |
| AAY78603 | BnaC08g01630D (brassica napus) CDX86540 | | 88% |
| AtFRK4 | SIFRK2 (solanum lycopersicum) NP_001233888 | Cytosol | 84% |
| At3g59480 | FRK4 (camelina sativa) XP_010469311 | | 97% |
| CAB75445 | BnaC08g29670D (brassica napus) CDX71907 | | 95% |
| | BnaA07g18380D (brassica napus) CDX67798 | | 95% |
| | FRK4 (glycine max) XP_003543564 | | 83% |
| | FRK4 (Noccaea caerulescens) JAU27780 | | 98% |
| | FRK4 (brassica rapa) XP_009116612 | | 95% |
| | FRK4 (brassica oleracea) XP_013600923 | | 96% |
| | FRK4 (raphanus sativus) XP_018460715 | | 94% |
| | FRK4 (citrus sinensis) XP_015386006 | | 86% |
| | FRK4 (ziziphus jujube) XP_015886997 | | 87% |
| | FRK4 (vitis vinifera) XP_002268097 | | 84% |
| | FRK4 (populous euphratica) XP_011037105 | | 85% |
| AtFRK5 | SIFRK2 (solanum lycopersicum) NP_001233888 | Cytosol | 70% |
| At4g10260 | FRK5 (camelina sativa) XP_010421945 | | 99% |
| CAB78149 | BnaC03g28870D (brassica napus) CDY05089 | | 97% |
| | FRK5 (populous euphratica) XP_010999528 | | 81% |
| | FRK5 (prunus mume) XP_008237799 | | 83% |
| | FRK5 (solanum tuberosum) XP_006362721 | | 81% |
| | FRK5 (glycine max) XP_003535409 | | 77% |
| | FRK2 (zea maize) ACG34312 | | 75% |
| | FRK5 (brassica rapa) XP_009134185 | | 98% |
| | FRK5 (brassica oleracea) XP_013627123 | | 97% |
| | FRK5 (raphanus sativus) XP_018475099 | | 96% |
| | FRK5 (citrus sinensis) XP_006478288 | | 81% |
| | FRK5 (ziziphus jujube) XP_015889502 | | 83% |
| | FRK5 (vitis vinifera) XP_002263733 | | 82% |
| | FRK5 (nicotiana silvestris) XP_009783705 | | 80% |
| | FRK5 (cicer arietnum) XP_004495435 | | 76% |
| | FRK5 (citrus clementina) XP_006441854 | | 81% |
| | FRK5 (juglans regia) XP_018834006 | | 75% |
| | FRK5 (fragaria vesca) XP_004290279 | | 82% |
| | FRK5 (ricinus communis) XP_002521094 | | 83% |
| | FRK5 (jatropha curcas) XP_012068437 | | 81% |
| | FRK5 (malus domestica) XP_017179574 | | 83% |
| | FRK5 (sesamum indicum) XP_011074982 | | 77% |
| | FRK5 (Theobroma cacao) XP_017980783 | | 76% |
| | FRK2 (oryza sativa) XP_018834006 | | 77% |
| | FRK2 (ananas comosus) OAY64795 | | 77% |
| | FRK5 (Gossypium raimondii) XP_012493063 | | 77% |
| AtFRK6 | SIFRK3 (solanum lycopersicum) NP_001234396 | Plastid | 78% |
| At1g66430 | FRK6 (camelina sativa) XP_010511466 | | 92% |
| AAG51160 | FRK6 (brassica napus) XP_013649151 | | 91% |
| | FRK6 (populous euphratica) XP_011002780 | | 78% |
| | FRK6 (prunus mume) XP_008229214 | | 77% |
| | FRK6 (malus domestica) XP_008378129 | | 76% |
| | FRK6 (capsicum annum) XP_016560842 | | 77% |
| | FRK6 (cucumis melo) XP_008457065 | | 76% |
| | hypothetical protein (capsella rubella) XP_006302390 | | 89% |
| | FRK6 (Noccaea caerulescens) JAU06553 | | 89% |
| | FRK6 (brassica rapa) XP_009105206 | | 90% |
| | FRK6 (brassica oleracea) XP_013591850 | | 90% |
| | BnaA07g25850D (brassica napus) CDX96146 | | 90% |
| | FRK6 (raphanus sativus) XP_018446898 | | 89% |
| | FRK6 (Pyrus × bretschneideri) XP_009334417 | | 75% |
| | FRK6 (cicer arietnum) XP_004490806 | | 76% |
| | FRK6 (sesamum indicum) XP_011099474 | | 81% |
| | FRK6 (jatropha curcas) XP_012077784 | | 78% |
| | FRK6 (Gossypium raimondii) XP_012471796 | | 80% |
| | FRK6 (eucaliptus grandis) XP_010062247 | | 77% |
| AtFRK7 | SIFRK1 (solanum lycopersicum) NP_001233893 | Cytosol | 74% |
| At5g51830 | FRK7 (brassica napus) XP_013640561 | | 94% |
| AAL34211 | FRK7 (prunus mume) XP_008234319 | | 82% |
| | FRK7 (malus domestica) XP_008376501 | | 82% |
| | FRK7 (capsella rubella) XP_006280740 | | 97% |
| | FRK7 (Noccaea caerulescens) JAU10903 | | 96% |
| | FRK7 (brassica rapa) XP_009132586 | | 95% |
| | FRK7 (brassica oleracea) XP_013612950 | | 93% |
| | FRK7 (raphanus sativus) XP_018457598 | | 92% |
| | BnaC03g15850D (brassica napus) CDY32617 | | 93% |
| | FRK7 (juglans regia) XP_018830828 | | 81% |
| | FRK7 (Pyrus × bretschneideri) XP_009371257 | | 82% |
| | FRK7 (Theobroma cacao) XP_007038535 | | 78% |
| | FRK7 (Gossypium raimondii) XP_012486482 | | 81% |
| | FRK7 (vitis vinifera) XP_002272526 | | 81% |
| | FRK7 (fragaria vesca) XP_004308129 | | 79% |
| | FRK7 (nicotiana tabacum) XP_016458421 | | 75% |
| | FRK7 (capsicum annum) XP_016564732 | | 75% |
| | FRK7 (glycine max) XP_003555364 | | 78% |
| | FRK7 (vigna radiata) XP_014506464 | | 78% |
| | hypothetical protein (citrus clementina) XP_006421810 | | 80% |

Thus, according to an aspect modifying oil content, germination rate and modulating activity or expression refers to increasing.

According to an embodiment of the invention, increasing expression of (or expressing) an FRK is performed by methods which are well known in the art.

According to an embodiment of the invention a nucleic acid sequence (polynucleotide) encoding the FRK is introduced into the plant.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

The term “isolated” refers to at least partially separated from the natural environment e.g., from a plant cell.

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

Nucleic acid sequences encoding the polypeptides of the present invention may be optimized for expression. Examples of such sequence modifications include, but are not limited to, an altered G/C content to more closely approach that typically found in the plant species of interest, and the removal of codons atypically found in the plant species commonly referred to as codon optimization.

The phrase “codon optimization” refers to the selection of appropriate DNA nucleotides for use within a structural gene or fragment thereof that approaches codon usage within the plant of interest. Therefore, an optimized gene or nucleic acid sequence refers to a gene in which the nucleotide sequence of a native or naturally occurring gene has been modified in order to utilize statistically-preferred or statistically-favored codons within the plant. The nucleotide sequence typically is examined at the DNA level and the coding region optimized for expression in the plant species determined using any suitable procedure, for example as described in Sardana et al. (1996, Plant Cell Reports 15:677-681). In this method, the standard deviation of codon usage, a measure of codon usage bias, may be calculated by first finding the squared proportional deviation of usage of each codon of the native gene relative to that of highly expressed plant genes, followed by a calculation of the average squared deviation. The formula used is: 1 SDCU=n=1 N[(Xn−Yn)/Yn] 2/N, where Xn refers to the frequency of usage of codon n in highly expressed plant genes, where Yn to the frequency of usage of codon n in the gene of interest and N refers to the total number of codons in the gene of interest. A Table of codon usage from highly expressed genes of dicotyledonous plants is compiled using the data of Murray et al. (1989, Nuc Acids Res. 17:477-498).

One method of optimizing the nucleic acid sequence in accordance with the preferred codon usage for a particular plant cell type is based on the direct use, without performing any extra statistical calculations, of codon optimization Tables such as those provided on-line at the Codon Usage Database through the NIAS (National Institute of Agrobiological Sciences) DNA bank in Japan (kazusa (dot) or (dot) jp/codon/). The Codon Usage Database contains codon usage tables for a number of different species, with each codon usage Table having been statistically determined based on the data present in Genbank.

By using the above methods to determine the most preferred or most favored codons for each amino acid in a particular species (for example, rice), a naturally-occurring nucleotide sequence encoding a protein of interest can be codon optimized for that particular plant species. This is effected by replacing codons that may have a low statistical incidence in the particular species genome with corresponding codons, in regard to an amino acid, that are statistically more favored. However, one or more less-favored codons may be selected to delete existing restriction sites, to create new ones at potentially useful junctions (5′ and 3′ ends to add signal peptide or termination cassettes, internal sites that might be used to cut and splice segments together to produce a correct full-length sequence), or to eliminate nucleotide sequences that may negatively affect mRNA stability or expression.

The naturally-occurring encoding nucleotide sequence may already, in advance of any modification, contain a number of codons that correspond to a statistically-favored codon in a particular plant species. Therefore, codon optimization of the native nucleotide sequence may comprise determining which codons, within the native nucleotide sequence, are not statistically-favored with regards to a particular plant, and modifying these codons in accordance with a codon usage table of the particular plant to produce a codon optimized derivative. A modified nucleotide sequence may be fully or partially optimized for plant codon usage provided that the protein encoded by the modified nucleotide sequence is produced at a level higher than the protein encoded by the corresponding naturally occurring or native gene. Construction of synthetic genes by altering the codon usage is described in for example PCT Patent Application 93/07278.

The invention also encompasses fragments of the above described FRKs and polypeptides having mutations, such as deletions, insertions or substitutions of one or more amino acids, either naturally occurring or man induced, either randomly or in a targeted fashion.

According to some embodiments of the invention, there is provided a plant cell exogenously expressing the polynucleotide (or in which the FRK expression has been modulated) of some embodiments of the invention, the nucleic acid construct of some embodiments of the invention and/or the polypeptide of some embodiments of the invention.

According to some embodiments of the invention, expressing the exogenous polynucleotide(s) e.g., at least one FRK e.., at least two FRKs, of the invention (or any other protein or nucleic acid sequence according to the present teachings) within the plant is effected by transforming one or more cells of the plant with the exogenous polynucleotide, followed by generating a mature plant from the transformed cells and cultivating the mature plant under conditions suitable for expressing the exogenous polynucleotide within the mature plant.

According to some embodiments of the invention, the transformation is effected by introducing to the plant cell a nucleic acid construct which includes the exogenous polynucleotide of some embodiments of the invention and at least one promoter for directing transcription of the exogenous polynucleotide in a host cell (a plant cell). Further details of suitable transformation approaches are provided herein below.

As mentioned, the nucleic acid construct according to some embodiments of the invention comprises a promoter sequence (e.g., developmentally regulated promoter active specifically in the embryo) and the isolated polynucleotide of some embodiments of the invention (e.g., FRK or at least two FRKs).

Thus, according to some embodiments of the invention, the polynucleotide encoding FRK (or any other polypeptide which is necessary of the present teachings e.g., Cas9) is operably linked to the promoter sequence.

A coding nucleic acid sequence is “operably linked” to a regulatory sequence (e.g., promoter) if the regulatory sequence is capable of exerting a regulatory effect on the coding sequence linked thereto.

As used herein, the term “promoter” refers to a region of DNA which lies upstream of the transcriptional initiation site of a gene to which RNA polymerase binds to initiate transcription of RNA. The promoter controls where (e.g., which portion of a plant) and/or when (e.g., at which stage or condition in the lifetime of an organism) the gene is expressed.

According to some embodiments of the invention, the promoter is heterologous to the isolated polynucleotide and/or to the host cell.

As used herein the phrase “heterologous promoter” refers to a promoter from a different species or from the same species but from a different gene locus as of the isolated polynucleotide sequence.

According to some embodiments of the invention, the isolated polynucleotide is heterologous to the plant cell (e.g., the polynucleotide is derived from a different plant species when compared to the plant cell, thus the isolated polynucleotide and the plant cell are not from the same plant species).

Any suitable promoter sequence can be used by the nucleic acid construct of the present invention. Preferably the promoter is a constitutive promoter, a tissue-specific, or an abiotic stress-inducible promoter.

According to some embodiments of the invention, the promoter is a plant promoter, which is suitable for expression of the exogenous polynucleotide in a plant cell.

Suitable constitutive promoters include, for example, CaMV 35S promoter [(CaMV 35S (pQXNc) Promoter); (PJJ 35S from Brachypodium); (CaMV 35S (OLD) Promoter) (Odell et al., Nature 313:810-812, 1985)]; maize Ub1 Promoter [cultivar Nongda 105; GenBank: DQ141598.1; Taylor et al., Plant Cell Rep 1993 12: 491-495, which is fully incorporated herein by reference; and cultivar B73; Christensen, A H, et al. Plant Mol. Biol. 18 (4), 675-689 (1992), which is fully incorporated herein by reference]; rice actin 1 (McElroy et al., Plant Cell 2:163-171, 1990); pEMU (Last et al., Theor. Appl. Genet. 81:581-588, 1991); CaMV 19S (Nilsson et al., Physiol. Plant 100:456-462, 1997); rice GOS2 [(rice GOS2 longer Promoter) and (rice GOS2 Promoter), de Pater et al, Plant J Nov;2(6):837-44, 1992]; RBCS promoter; Rice cyclophilin (Bucholz et al, Plant Mol Biol. 25(5):837-43, 1994); Maize H3 histone (Lepetit et al, Mol. Gen. Genet. 231: 276-285, 1992); Actin 2 (An et al, Plant J. 10(1); 107-121, 1996) and Synthetic Super MAS (Ni et al., The Plant Journal 7: 661-76, 1995). Other constitutive promoters include those in U.S. Pat. Nos. 5,659,026, 5,608,149; 5.608,144; 5,604,121; 5.569,597: 5.466,785; 5,399,680; 5,268,463; and 5,608,142.

Suitable tissue-specific promoters include, but not limited to, leaf-specific promoters [e.g., AT5G06690 (Thioredoxin) (high expression), AT5G61520 (AtSTP3) (low expression) described in Buttner et al 2000 Plant, Cell and Environment 23, 175-184, or the promoters described in Yamamoto et al., Plant J. 12:255-265, 1997; Kwon et al., Plant Physiol. 105:357-67, 1994; Yamamoto et al., Plant Cell Physiol. 35:773-778, 1994; Gotor et al., Plant J. 3:509-18, 1993; Orozco et al., Plant Mol. Biol. 23:1129-1138, 1993; and Matsuoka et al., Proc. Natl. Acad. Sci. USA 90:9586-9590, 1993; as well as Arabidopsis STP3 (AT5G61520) promoter (Buttner et al., Plant, Cell and Environment 23:175-184, 2000)], seed-preferred promoters [e.g., Napin (originated from Brassica napus which is characterized by a seed specific promoter activity; Stuitje A. R. et. al. Plant Biotechnology Journal 1 (4): 301-309; (Brassica napus NAPIN Promoter) from seed specific genes (Simon, et al., Plant Mol. Biol. 5. 191, 1985; Scofield, et al., J. Biol. Chem. 262: 12202, 1987; Baszczynski, et al., Plant Mol. Biol. 14: 633, 1990), rice PG5a (US 7,700,835), early seed development Arabidopsis BAN (AT1G61720) (US 2009/0031450 A1), late seed development Arabidopsis ABI3 (AT3G24650) (Arabidopsis ABI3 (AT3G24650) longer Promoter) or 10661 (Arabidopsis ABI3 (AT3G24650) Promoter)) (Ng et al., Plant Molecular Biology 54: 25-38, 2004), Brazil Nut albumin (Pearson' et al., Plant Mol. Biol. 18: 235- 245, 1992), legumin (Ellis, et al. Plant Mol. Biol. 10: 203-214, 1988), Glutelin (rice) (Takaiwa, et al., Mol. Gen. Genet. 208: 15-22, 1986; Takaiwa, et al., FEBS Letts. 221: 43-47, 1987), Zein (Matzke et al Plant Mol Biol, 143:323-32 1990), napA (Stalberg, et al, Planta 199: 515-519, 1996), Wheat SPA; Albanietal, Plant Cell, 9: 171-184, 1997), sunflower oleosin (Cummins, et al., Plant Mol. Biol. 19: 873- 876, 1992)], endosperm specific promoters [e.g., wheat LMW (Wheat LMW Longer Promoter), and (Wheat LMW Promoter) and HMW glutenin-1 [(Wheat HMW glutenin-1 longer Promoter)); and (Wheat HMW glutenin-1 Promoter), Thomas and Flavell, The Plant Cell 2:1171-1180, 1990; Mol Gen Genet 216:81-90, 1989; NAR 17:461-2), wheat alpha, beta and gamma gliadins ((wheat alpha gliadin (B genome) promoter); (wheat gamma gliadin promoter); EMBO 3:1409-15, 1984), Barley ltrl promoter, barley B1, C, D hordein (Theor Appl Gen 98:1253-62, 1999; Plant J 4:343-55, 1993; Mol Gen Genet 250:750-60, 1996), Barley DOF (Mena et al, The Plant Journal, 116(1): 53-62, 1998), Biz2 (EP99106056.7), Barley SS2 (Barley SS2 Promoter); Guerin and Carbonero Plant Physiology 114: 1 55-62, 1997), wheat Tarp60 (Kovalchuk et al., Plant Mol Biol 71:81-98, 2009), barley D-hordein (D-Hor) and B-hordein (B-Hor) (Agnelo Furtado, Robert J. Henry and Alessandro Pellegrineschi (2009)], Synthetic promoter (Vicente-Carbajosa et al., Plant J. 13: 629-640, 1998), rice prolamin NRP33, rice -globulin Glb-1 (Wu et al, Plant Cell Physiology 39(8) 885-889, 1998), rice alpha-globulin REB/OHP-1 (Nakase et al. Plant Mol. Biol. 33: 513-S22, 1997), rice ADP-glucose PP (Trans Res 6:157-68, 1997), maize ESR gene family (Plant J 12:235-46, 1997), sorgum gamma-kafirin (PMB 32:1029-35, 1996)] and flower-specific promoters [e.g., AtPRP4, chalene synthase (chsA) (Van der Meer, et al., Plant Mol. Biol. 15, 95-109, 1990), LAT52 (Twell et al Mol. Gen Genet. 217:240-245; 1989), Arabidopsis apetala-3 (Tilly et al., Development. 125:1647-57, 1998), Arabidopsis APETALA 1 (AT1G69120, AP1) (Arabidopsis (AT1G69120) APETALA 1)) (Hempel et al., Development 124:3845-3853, 1997)], and root promoters [e.g., the ROOTP promoter; rice ExpB5 (rice ExpB5 Promoter); or (rice ExpB5 longer Promoter)) and barley ExpB1 promoter (Won et al. Mol. Cells 30: 369-376, 2010); arabidopsis ATTPS-CIN (AT3G25820) promoter; Chen et al., Plant Phys 135:1956-66, 2004); arabidopsis Phol promoter (Hamburger et al., Plant Cell. 14: 889-902, 2002), which is also slightly induced by stress].

According to a specific embodiment, the promoter is a developmentally regulated promoter, active specifically in the embryo, that is also referred to as an “embryo specific promoter” [e.g., rice OSH1 (Sato et al, Proc. Natl. Acad. Sci. USA, 93: 8117-8122), KNOX (Postma-Haarsma et al, Plant Mol. Biol. 39:257-71, 1999), rice oleosin (Wu et at, J. Biochem., 123:386, 1998)]. Other genes with high specific expression in embryos include, but are not limited to, Cruciferin 3 (At4g28520), AT3G63040, AT4G36700, AT1G62290, AT1G47540, AT1G65090, AT1G54860 and At3g6340.

It will be appreciated that various construct schemes can be utilized to express both FRKs from a single nucleic acid construct.

For example, the two FRKs can be co-transcribed as a polycistronic message from a single promoter sequence of the nucleic acid construct. To enable co-translation of both FRKs from a single polycistronic message, the first and second polynucleotide segments can be transcriptionally fused via a linker sequence including an internal ribosome entry site (IRES) sequence which enables the translation of the polynucleotide segment downstream of the IRES sequence. In this case, a transcribed polycistronic RNA molecule including the coding sequences of both the first and the second FRKs will be translated from both the capped 5′ end and the internal IRES sequence of the polycistronic RNA molecule to thereby produce both the first and the second FRKs.

According to some embodiments of the invention, over-expression of the FRK of the invention is achieved by means of genome editing.

Over expression of a polypeptide by genome editing can be achieved by: (i) replacing an endogenous sequence encoding the polypeptide of interest or a regulatory sequence under which it is placed, and/or (ii) inserting a new gene encoding the polypeptide of interest in a targeted region of the genome, and/or (iii) introducing point mutations which result in up-regulation of the gene encoding the polypeptide of interest (e.g., by altering the regulatory sequences such as promoter, enhancers, 5′-UTR and/or 3′-UTR, or mutations in the coding sequence).

Methods for genome editing are further exemplified hereinbelow. Decreasing oil content/seed germination rate is achieved by decreasing (down-regulating) FRK activity of expression. It will be appreciated that decreasing oil content may be desired for nutritional reasons, accumulate higher relative protein levels, higher relative sugar (e.g. sucrose, fructose, erythritol, maltose) levels, higher levels of different nutrients (e.g. free amino acids such as methionine, threonine, valine and isoleucine) and ease of processing.

As used herein the phrase “dowregulates expression” refers to dowregulating the expression of a protein (e.g. FRK or at least two FRKs) at the genomic (e.g. homologous recombination and site specific endonucleases) and/or the transcript level using a variety of molecules which interfere with transcription and/or translation (e.g., RNA silencing agents) or on the protein level (e.g., aptamers, small molecules and inhibitory peptides, antagonists, enzymes that cleave the polypeptide, antibodies and the like).

For the same culture conditions the expression is generally expressed in comparison to the expression in a cell of the same species but not subjected to modulation of FRK activity or expression, also referred to as control.

Down regulation of expression (or over-expression, as described above) may be either transient or permanent.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically, a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is FokI. Additionally Fokl has the advantage of requiring dimerization to have nuclease activity and this means the specificity increases dramatically as each nuclease partner recognizes a unique DNA sequence. To enhance this effect, FokI nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fokl domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2- His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May;30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, Calif.).

Another agent capable of downregulating FRK and/or any other gene of the mentioned loci is a RNA-guided endonuclease technology e.g. CRISPR system.

As used herein, the term “CRISPR system” also known as Clustered Regularly Interspaced Short Palindromic Repeats refers collectively to transcripts and other elements involved in the expression of or directing the activity of CRISPR-associated genes, including sequences encoding a Cas9 gene (e.g. CRISPR-associated endonuclease 9), a tracr (trans-activating CRISPR) sequence (e.g. tracrRNA or an active partial tracrRNA), a tracr-mate sequence (encompassing a “direct repeat” and a tracrRNA-processed partial direct repeat) or a guide sequence (also referred to as a “spacer”) including but not limited to a crRNA sequence (i.e. an endogenous bacterial RNA that confers target specificity yet requires tracrRNA to bind to Cas) or a sgRNA sequence (i.e. single guide RNA).

In some embodiments, one or more elements of a CRISPR system is derived from a type I, type II, or type III CRISPR system. In some embodiments, one or more elements of a CRISPR system (e.g. Cas) is derived from a particular organism comprising an endogenous CRISPR system, such as Streptococcus pyogenes, Neisseria meningitides, Streptococcus thermophilus or Treponema denticola.

In general, a CRISPR system is characterized by elements that promote the formation of a CRISPR complex at the site of a target sequence (also referred to as a protospacer in the context of an endogenous CRISPR system).

In the context of formation of a CRISPR complex, “target sequence” refers to a sequence to which a guide sequence (i.e. guide RNA e.g. sgRNA or crRNA) is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. Thus, according to some embodiments, global homology to the target sequence may be of 50%, 60%, 70%, 75%, 80%, 85%, 90%, 95% or 99%. A target sequence may comprise any polynucleotide, such as DNA or RNA polynucleotides. In some embodiments, a target sequence is located in the nucleus or cytoplasm of a cell.

Thus, the CRISPR system comprises two distinct components, a guide RNA (gRNA) that hybridizes with the target sequence, and a nuclease (e.g. Type-II Cas9 protein), wherein the gRNA targets the target sequence and the nuclease (e.g. Cas9 protein) cleaves the target sequence. The guide RNA may comprise a combination of an endogenous bacterial crRNA and tracrRNA, i.e. the gRNA combines the targeting specificity of the crRNA with the scaffolding properties of the tracrRNA (required for Cas9 binding). Alternatively, the guide RNA may be a single guide RNA capable of directly binding Cas.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence.

In some embodiments, the tracr sequence has sufficient complementarity to a tracr mate sequence to hybridize and participate in formation of a CRISPR complex. As with the target sequence, a complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned.

Introducing CRISPR/Cas into a cell may be effected using one or more vectors driving expression of one or more elements of a CRISPR system such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. A single promoter may drive expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron).

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, transformed into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After transformation and positive selection, homologously targeted clones are identified. Next, a second targeting vector that contains a region of homology with the desired mutation is transformed into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Silencing at the transcript (RNA) level can be effected using the below exemplary platforms.

As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene (e.g., AtFRK6 and AtFRK7). In certain embodiments, the RNA silencing agent is capable of preventing complete processing (e.g, the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA (e.g., AtFRK6 and AtFRK7) and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Following is a detailed description on RNA silencing agents that can be used according to specific embodiments of the present invention.

DsRNA, siRNA and shRNA—The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment dsRNA longer than 30 bp are used. Various studies demonstrate that long dsRNAs can be used to silence gene expression without inducing the stress response or causing significant off-target effects - see for example [Strat et al., Nucleic Acids Research, 2006, Vol. 34, No. 13 3803-3810; Bhargava A et al. Brain Res. Protoc. 2004;13:115-125; Diallo M., et al., Oligonucleotides. 2003;13:381-392; Paddison P.J., et al., Proc. Natl Acad. Sci. USA. 2002;99:1443-1448; Tran N., et al., FEBS Lett. 2004;573:127-134].

According to some embodiments of the invention, dsRNA is provided in cells where the interferon pathway is not activated, see for example Billy et al., PNAS 2001, Vol 98, pages 14428-14433. and Diallo et al, Oligonucleotides, Oct. 1, 2003, 13(5): 381-392. doi:10.1089/154545703322617069.

According to an embodiment of the invention, the long dsRNA are specifically designed not to induce the interferon and PKR pathways for down-regulating rgene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 base pairs) that induce the RNA interference (RNAi) pathway. Typically, siRNAs are chemically synthesized as 2lmers with a central 19 bp duplex region and symmetric 2-base 3′-overhangs on the termini, although it has been recently described that chemically synthesized RNA duplexes of 25-30 base length can have as much as a 100-fold increase in potency compared with 2lmers at the same location. The observed increased potency obtained using longer RNAs in triggering RNAi is suggested to result from providing Dicer with a substrate (27mer) instead of a product (21mer) and that this improves the rate or efficiency of entry of the siRNA duplex into RISC.

It has been found that position of the 3′-overhang influences potency of a siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript. The strands of a double-stranded interfering RNA (e.g., a siRNA) may be connected to form a hairpin or stem-loop structure (e.g., a shRNA). Thus, as mentioned, the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. W02013126963 and W02014107763). It will be recognized by one of skill in the art that the resulting single chain oligonucleotide forms a stem-loop or hairpin structure comprising a double-stranded region capable of interacting with the RNAi machinery.

Synthesis of RNA silencing agents suitable for use with some embodiments of the invention can be effected as follows. First, the FRK (e.g., AtFRK6 and AtFRK7) mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www(dot)ambion(dot)com/techlib/tn/91/912(dot)html).

Second, potential target sites are compared to an appropriate genomic database (e.g., human, mouse, rat etc.) using any sequence alignment software, such as the BLAST software available from the NCBI server (www(dot)ncbi(dot)nlm(dot)nih(dot)gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

Constructs useful in the methods according to the present invention may be constructed using recombinant DNA technology well known to persons skilled in the art. The coding sequence (e.g., encoding a silencing agent to the target sequences may be inserted into vectors, which may be commercially available, suitable for transforming into plants and suitable for expression of the gene of interest in the transformed cells. The genetic construct can be an expression vector wherein the nucleic acid sequence is operably linked to one or more regulatory sequences allowing expression in the plant cells.

The term “plant” as used herein encompasses a whole plant, a grafted plant, ancestor(s) and progeny of the plants and plant parts, including seeds, shoots, stems, roots (including tubers), rootstock, scion, and plant cells, tissues and organs. The plant may be in any form including suspension cultures, embryos, meristematic regions, callus tissue, leaves, gametophytes, sporophytes, pollen, and microspores. Plants that are particularly useful in the methods of the invention include all plants which belong to the superfamily Viridiplantae, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalypfus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medicago saliva, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, maize, wheat, barley, rye, oat, peanut, pea, lentil and alfalfa, cotton, rapeseed, canola, pepper, sunflower, tobacco, eggplant, eucalyptus, a tree, an ornamental plant, a perennial grass and a forage crop. Alternatively algae and other non-Viridiplantae can be used for the methods of the present invention.

According to some embodiments of the invention, the plant is a crop plant such as rice, maize, wheat, barley, peanut, potato, sesame, olive tree, palm oil, banana, soybean, sunflower, canola, sugarcane, alfalfa, millet, leguminosae (bean, pea), flax, lupinus, rapeseed, tobacco, poplar and cotton.

According to some embodiments of the invention the plant is a dicotyledonous plant.

According to some embodiments of the invention the plant is a monocotyledonous plant.

According to some embodiments, the plant is an oil seed plant i.e., in which the endosperm is substantially based on oil and not on starch for instance.

Examples of oil seed plants include, but are not limited to castor, rapeseed, soybean, cotton, poppy, sunflower, sesame, peanut, coconut, almond, jojoba, mustard, avocado and olive.

According to a specific embodiment, the plant is not a tree.

Plant cells may be transformed stably or transiently with the nucleic acid constructs of the present invention. In stable transformation, the nucleic acid molecule of the present invention is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principle methods of causing stable integration of exogenous DNA into plant genomic DNA include two main approaches:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112;

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217.

Glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledonous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

However other methods of production are also contemplated including sexual reproduction (and selection for the determinate phenotype whether morphologically or using molecular markers as described herein), tissue culture and more.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet gradually increased so that it can be grown in the natural environment.

Viruses that have been shown to be useful for the transformation of plant hosts include CaMV, TMV, TRV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

It will be appreciated that expression (or the modulation of FRK expression) can be in a homozygous or a heterozygous form.

As mentioned hereinabove, seeds having modified oil content or modified germination rate can be selected.

Marker assisted breeding can be employed whereby FRK or expression thereof can be determined in the DNA, RNA or polypeptide level using methods which are well known in the art.

Assays for measuring seed oil content/composition are well known in the art and include, measuring seed mass (typically determined on thousand seed), seed anatomy, fatty acid composition-that may be determine using various spectroscopic as well as chromatographic methods, e.g., gas-chromatography.

The selected seeds may be germinated and may be the subject for further modifications or crossings (e.g., with elite lines).

Thus, the present teachings provide for plants e.g., an oil seed plant having been genetically modified or selected to modulate FRK activity in seeds thereof.

Also provided is an oil seed plant obtainable according to the methods as described herein (e.g., characterized by increased FRK activity as compared to a control plant described hereinabove).

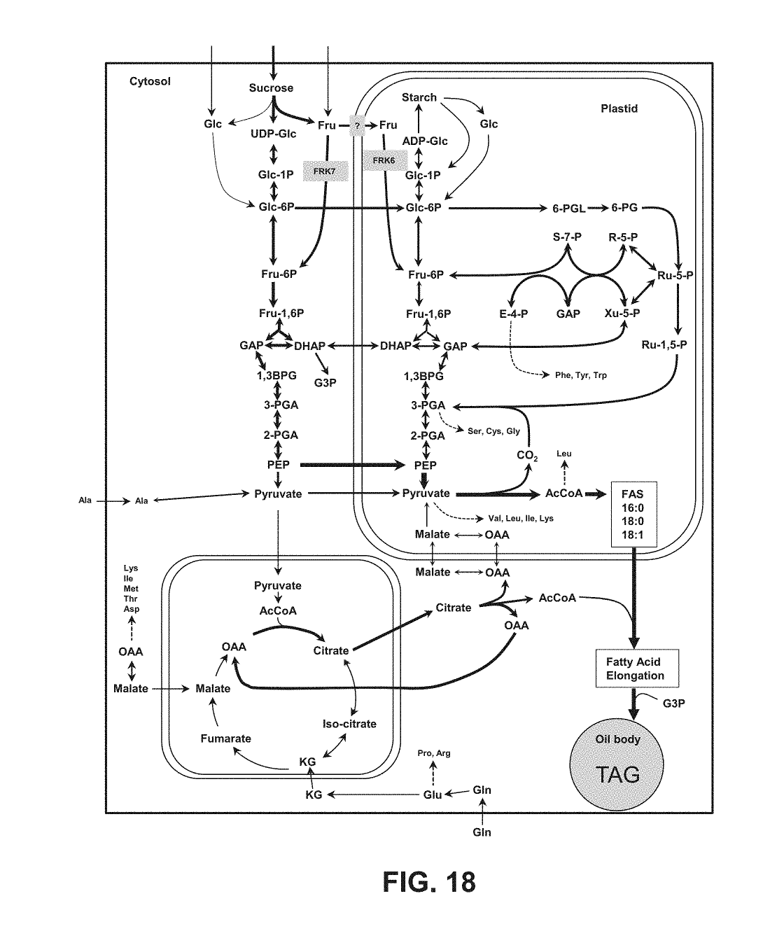

Also provided is a plant part of the plant as described herein e.g., seed and further oil of the plant. Such oil may have a different oil composition such as an increased TCA cycle metabolites and/or larger oil bodies as compared to oil of a plant of the same species not subjected to the FRK modulation as described herein (see FIG. 18).

Also provided is a method of producing oil, the method comprising:

providing seeds of a plant having a modulated FRK activity (e.g., increased); and

extracting oil from said seeds.

It will be appreciated that the amount and/or quality of the oil of some embodiments render the methods of producing oil specifically attractive i.e., less seed is required to obtain a given amount of oil as compared to seed of the control plant.

The present teachings also refer to a cake of such plants, that may have a distinct chemical composition.

It is expected that during the life of a patent maturing from this application many relevant FRKs will be discovered and the scope of the term FRK is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, a given SEQ ID NO: is expressed in a DNA sequence format (e.g., reciting T for thymine), but it can refer to either a DNA sequence that corresponds to a given nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### MATERIALS AND METHODS

Plant Material, Growth Conditions and Sugar Treatments

Arabidopsis (Arabidopsis thaliana) wild-type plants, T-DNA-tagged mutant plants and transgenic plants were of the Col-0 ecotype. Mutant seeds with a T-DNA insertion in their AtFRK genes were obtained from the Arabidopsis Biological Resource Center and are listed in Table 2 below.

TABLE 2

| Gene name | Gene ID | Uniprot ID | Uniprot annotation | T-DNA line | mutant name |
|---|---|---|---|---|---|
| AtFRK1 | At2g31390 | Q9SID0 | probable FRK1 | SALK_114786 | frk1 |
| AtFRK2 | At1g06030 | Q9LNE3 | probable FRK2 | none | |
| AtFRK3 | At1g06020 | Q9LNE4 | probable FRK3 | SALK_122966 | frk3 |
| AtFRK4 | At3g59480 | Q9M1B9 | probable FRK4 | SALK_058121 | frk4 |
| AtFRK5 | At4g10260 | O82616 | probable FRK5 | none | |
| AtFRK6 | At1g66430 | Q9C524 | probable FRK6 | SALK_142725 | frk6 |
| AtFRK7 | At5g51830 | Q9FLH8 | probable FRK7 | SALK_046463 | frk7 |

Seeds were sown in soil or sterilized and sown on half-strength Murashige and Skoog (MS) medium (Murashige & Skoog, 1962) with or without 1% sucrose, glucose, fructose or mannitol. Seeds were kept at 4° C. for 3 d in the dark for stratification and then transferred to normal growth conditions. Plants were grown in a walk-in growth chamber and kept at 22° C. with a 16-h light/8-h dark photoperiod unless stated otherwise.

Vector Construction and Plant Transformation

The FRK1 cDNA (SlFRK1) from tomato (Solanum lycopersicum L.; GenBank accession number U64817, SEQ ID NO: 106 and 107) was inserted in the sense orientation between the cauliflower mosaic virus 35S promoter and the nopaline-synthase termination site in the binary vector pBI121 (Odanaka et al., 2002). The beta-glucuronidase gene in pBI121 was removed by digestion with BamHI and SacI, and was replaced by FRK1 cDNA including ˜270 bp of the 5′ untranslated region and ˜50 bp of the 3′ untranslated region. This FRK1 vector was introduced into Agrobacterium tumefaciens for the transformation. Agrobacterium-mediated transformation of Arabidopsis thaliana was performed using the floral-dip method as described previously (Clough & Bent, 1998).

Seed Weight

Seeds of the WT, frk6, frk7 and the double-mutant were harvested from plants grown under even-day conditions (12-hour photoperiod) at the same time. One thousand two hundred to one thousand eight hundred seeds from three individual plants per line were photographed on a white paper and counted manually using ImageJ software (http://rsb(dot)info9(dot)nih(dot)gov/ij/). Seeds were then weighed using an analytical scale and that figure was divided by the number of seeds per plant to determine the average seed weight per plant.

DNA Extraction, RNA Extraction, cDNA Preparation and PCR Analysis

Genomic DNA was extracted as described previously (Edwards et al., 1991). Genotyping was done with right plus left primers flanking the T-DNA of each of the SALK lines and with right primer plus the LBb 1 primer for confirming existence of the T-DNA insertion. Primers used for PCR amplification are listed in Table 3.

TABLE 3

| | Sequence (5′-3′)/ |
|---|---|
| Primer name | SEQ ID NO: 108-132 |
| SALK_114786 L | TCTTACCAAAAGGCAAAACCC |
| SALK_114786 R | AAATTCAATAACGCGATTCCC |
| SALK_046463 L | CATGCGTTTGCAAATAGAAGC |
| SALK_046463 R | ACCAGCTTTCAAGAAAGCTCC |
| SALK_122966 L | CAGGAAGCTTGATGAGGAGTG |
| SALK_122966 R | CAAGATTCAGTTCATCTGGCC |
| SALK_058121 L | TCATACATGTCAAAGCTAGCC |
| SALK_058121 R | GCACTAGGGTTCCGGTAGAAC |
| SALK_142725 L | AACAACATATCAGCACTCGGG |
| SALK_142725 R | TTGAATGAGGTGATCGATGTG |
| LBb1 | GCGTGGACCGCTTGCTGCAACT |
| At2g31390 11 F | TTCTTCTCCTTGTCCTTCCTT |
| At2g31390 570 R | CACTTCCATGGCCTTCAAAT |
| At1g66430 25 F | CGCTCTCTCAGCTTCCTCTC |
| At1g66430 320 R | GGAGATTCTTGTGTTGAACCATC |
| At5g51830 335 F | ACTTGGTGGCTCCTCTGCT |
| At5g51830 627 R | GGCAAGGTTCCTCAATCAAA |
| At1g06020 233 F | GAAAGAACGGTGTCGACGAT |
| At1g06020 1040 R | TTAACAAGGAGAAGCCGTACA |
| At3g59480 1 F | ATTATATCCAAAACATAATCTCTCTC |
| At3g59480 690 R | AATCTGCTTCTGAGCCTCCT |
| AtHXK1 F | GCGGGAAGCAAGAGCGTGTT |
| AtHXK1 R | CTCCTCGGGTTGCTATGATG |
| S1FRK1 317 F | TAGAAATGGTGCGGTGTCAAA |
| S1FRK1 538 R | AACATACGGCCGAACTCATC |

Total RNA was extracted using the LogSpin method (Yaffe et al., 2012). Samples were ground using a Geno/grinder (SPEX SamplePrep, Metuchen, N.J., USA) and RNA was extracted in 8 M guanidine hydrochloride buffer (Duchefa Biochemie, Haarlem, The Netherlands) and transferred to tubes containing 96% EtOH (Bio Lab, Jerusalem, Israel). T hen, samples were transferred through a plasmid DNA extraction column (RBC Bioscience, New Taipei City, Taiwan), followed by two washes in 3 M Na-acetate (BDH Chemicals, Mumbai, India) and two washes in 75% EtOH and eluted with DEPC (diethylpyrocarbonate) water (Biological Industries Co., Beit Haemek, Israel) that had been preheated to 65° C. The RNA was treated with RQ1-DNase (ProMega, Madison, Wis., USA) according to the manufacturer's instructions, to degrade any residual DNA. For the preparation of cDNA, total RNA (1 μtg) was taken for reverse transcription-PCR using MMLV RT (ProMega) in a 25-μl reaction, with 2 μl of random primers (ProMega) and 1 μl of oligo-dT primers (ProMega). cDNA samples were diluted 1:4 in water. Amplification of the AtFRKs was done by PCR. Following an initial preheating step at 94° C. for 2 min, there were 37 cycles of amplification consisting of 20 s at 94° C., 20 s at 60° C. and 60 s at 72° C. The Arabidopsis AtHXKl (accession no. At4g29130) was used as a reference gene. Primers used for PCR amplification are listed in Table 3.

Scanning Electron Microscopy (SEM)

Dry seeds were attached to a metal stub with double-sided carbon tape and coated with gold palladium (Quorum SC7620 mini sputter coater). Images were taken with a JEOL JCM-6000 benchtop SEM. Analysis was performed using SEM software.

Extraction, Derivatization, and Analysis of Arabidopsis Seeds Primary Metabolites Using GC-MS

For each line, 40 mg of mature Arabidopsis seeds from six individual plants were carefully cleaned of debris and collected in 2-ml Eppendorf tubes. The samples were frozen in liquid nitrogen and ground using a Geno/grinder (SPEX SamplePrep, Metuchen, N.J., USA). The samples were extracted in 1 mL of methanol/chloroform/DDW solution (2.5/1/1) and 15 _{I}ll internal standard was added (0.2 mg ml⁻¹ ribitol in water). Following 1 h of shaking at 4° C., the samples were centrifuged for 10 min at 14,000 rpm and 900 μl of the supernatant were transferred to a new 1.5-ml tube. Five hundred μl DDW were added for phase separation and the upper polar phase was transferred to a new 1.5-ml tube and dried using a speed-vac before storage. Derivatization, standard addition and sample injection were exactly as described previously (Lisec et al., 2006).

The GC-MS system was comprised of a CTC CombiPAL autosampler, an Agilent 6890N gas chromatograph and a LECO Pegasus III TOF-MS running in EI+ mode. Metabolites were identified in comparison to database entries of authentic standards (Kopka et al., 2005). Chromatograms and mass spectra were evaluated using Chroma TOF 1.0 (LECO) and TagFinder 4.0 software (Luedemann et al., 2008). Results are provided in Table 4 below.

[symbol]

upplemental Table— [symbol] etabolite Reporting Guidelines (Checklist table)

| Level | Aspect | Information | Fill in |
|---|---|---|---|
| general | Type of metabolome analysis | targeted metabolite analysis | TRUE |
| aspect | | non-targeted metabolite class scale profiling | FALSE |
| | | non-targeted metabolome scale profiling | FALSE |
| | | non-targeted finger printing of mass features | FALSE |
| | Type of quantification | absolute or quantification | Relative quantcation |
| | Type of reference samples | chemically defined | standard reference compounds aquired in chemical companies |
| | | biologically defined | — |
| | Type of replication | analytical (same analytical sample preparation) | 1 |
| | | technological (same biological preparation) | 1 |
| | | biological (same experimental condition) | 6 |
| | | full experiment | 1 |
| | Type of tecnnology | reference pubilcation | Lisec et al (2006) |
| | Sample preparation | | chemical derivatized |
| | | method of chromatography/separation | Lisec et al (2006) |
| | | method of ionization | −70 MeVolt hard ionization |
| | | method of detection | electron impact ionization |
| metabolite/ | Metabolite | metabolite name | see table 2 |
| mass | | metabolite sum formula | see table 2 |
| feature | | metabolite structure and public source of metabolite identifier | Metabolites were identified in comparison to database entries of authentic standards |
| | | | (Kopka et al., 2005; Schauer et al., 2005). |
| | Identification | identification process | manually supervized with TagFinder software |
| | | by authentic mass isotopomer added to one or at biological sample(s) | FALSE |
| | | by authentic reference compound within a co-processed reference mixture | FALSE |
| | | by authentic reference compound previously mapped to the analytical | TRUE |
| | | system reference library | Metabolites were identified in comparison to database entries of authentic standards |
| | | | (Kopka et al., 2005; Schauer et al., 2005) |
| | | type of mass spectrum | |
| | | by match of molecular mass (single mass fragment) | A minimum of 3 unique mass fragments was required for the relative quantification |
| | | | of metabolite pool sizes |
| | | by match of fragments | YES |
| | | by match of fragmentation pattern | YES |
| | | by match of mass spectrum to reference library | Mass spectral matching thresholds were: Match > 650 (best match = 1000) and |
| | | | RI-deviation < 1.0% |
| | | type of retention index | |
| | | by match of retention time (index) to reference library | fatty acid methylesters (FAMEs) |
| | Quantification | type of quantification | relative quantification by internal standard and sample fresh weight |
| | Validity testing | Recovery testing (chemical analog) | not performed |
| | | Recovery testing (internally added mass isotopomer) | not performed |
| | | Recovery testing (mixture of most divergent samples from the experiment) | not performed |
| | | Test for linear range | not performed |
| | | Limit of quantification (LOQ) | not performed |
| | | Limit of detection (LOD) | not performed |

[symbol]

ol. Formula—molecular [symbol] ormula of the metabolite or its FA adduct;
Mass to charge ratio (m/z)
(S)-identification confined by a standard compound
I, II, III—different isomers
Identification level (A; B; C; D)-(A) standard or NMR; (B) MS/MS; (C) MS^{E}; (D) MS only

| Peak/ | | Tag | Time | | | | | Mass to | | | Identi- |
|---|---|---|---|---|---|---|---|---|---|---|---|
| Compound | Time | Time | Devi- | | Corresponding | Metabolite | | charge | Species | Refer- | fication |
| n [symbol] | Expe [symbol] | Ind [symbol] | ation | Putative metabolite name (Derivative) | Metaboli [symbol] | Class | Mol formula | ratio | detected before | ences | le [symbol] |
| 1 | 206867 | 206522 | −0.13 | M000517_A106002- | Glycolate | Organic acids | C₂H₄O₃ | 133 | Tomato, Arabidopsis | | A |
| | | | | 101_CONT-MST_206867_TOF_Glycolic acid (2TMS) | | | | | | | |
| 2 | 222650 | 222603 | -0.04 | M000071_A104002- | Pyruvate | Organic acids | C₃H₄O₃ | 174 | Tomato, Arabidopsis | | A |
| | | | | 101_METB_222650_TOF_Pyruvic acid (1MEOX) | | | | | | | |
| | | | | (1TMS) | | | | | | | |
| 3 | 271580 | 271672 | 0.04 | M000030_A122001- | Valine | Amino acids | C₅H₁₁NO₂ | 144 | Tomato, Arabidopsis, | | A |
| | | | | 101_METB_271580_TOF_Valine, DL-(2TMS) | | | | | potato, Lotus japonicus | | |
| 4 | 319193 | 319006 | −0.06 | M000017_A132002- | Isoleucine | Amino acids | C₆H₁₃NO₂ | 158 | Tomato, Arabidopsis, | | A |
| | | | | 101_METB_319193_TOF_Isoleucine, L-(2TMS) | | | | | potato | | |
| 5 | 325180 | 325093 | −0.03 | M000031_A133001- | Glycine | Amino acid | C₂H₅NO₂ | 174 | Tomato, Arabidopsis | | A |
| | | | | 101_METB_325180_TOF_Glycine (3TMS) | | | | | | | |
| 6 | 345050 | 344510 | −0.18 | M000073_A135003- | Glycerate | Organic acids | C₃H₅O₄ | 189 | Tomato, Arabidopsis | | A |
| | | | | 101_METB_345050_TOF_Glyceric acid, DL-(3TMS) | | | | | | | |
| 7 | 348220 | 348174 | −0.01 | M000347_A128003- | Benzoic acid | Organic acids | C₇H₆O₂ | 105 | Tomato, Arabidopsis | | A |
| | | | | 101_CONT-METB_348220_TOF_Benzoic acid (1TMS) | | | | | | | |
| 8 | 354060 | 353828 | −0.06 | M000026_A138002- | Alanine | Amino acids | C₃H₇NO₂ | 188 | Tomato, Arabidopsis, | | A |
| | | | | 101_METB_354060_TOF_Alanine, DL-(3TMS) | | | | | potato, Lotus japonicus | | |
| 9 | 357523 | 357212 | −0.1 | M000015_A138001- | Serine | Amino acids | C₃H₇NO₃ | 204 | Tomato, Arabidopsis, | | A |
| | | | | 101_METB_357523_TOF_Serine, DL-(3TMS) | | | | | potato | | |
| 10 | 368365 | 367285 | −0.31 | M000016_A140001- | Theonine | Amino acids | C₄H₈NO₃ | 218 | Tomato, Arabidopsis, | | A |
| | | | | 101_METB_368365_TOF_Threonine, DL-(3TMS) | | | | | potato | | |
| 11 | 371255 | 370927 | −0.05 | M000067_A137001- | Fumarate | Organic acids | C₄H₄O₄ | 245 | Tomato, Arabidopsis, | | A |
| | | | | 101_METB_371255_TOF_Fumaric acid (2TMS) | | | | | potato, Lotus japonicus | | |
| 12 | 386210 | 386315 | 0.03 | M000457_A133004- | Nicotinic acid | Organic acids | C₆H₅NO₂ | 136 | Tomato, Arabidopsis | | A |
| | | | | 101_METB_386210_TOF_Nicotinic acid (1TMS) | | | | | | | |
| 13 | 394250 | 393917 | −0.09 | M000027_A144001- | beta-alanine | Amino acids | C₃H₇NO₂ | 174 | Tomato, Arabidopsis, | | A |
| | | | | 101_METB_394250_TOF_Alanine, beta-(3TMS) | | | | | potato, Lotus japonicus | | |
| 14 | 408455 | 408238 | −0.06 | M000054_A150002- | Erythritol | Polyol | C₄H₁₀O₄ | 217 | Tomato, Arabidopsis, | | A |
| | | | | 101_METB_408455_TOF_Erythritol (4TMS) | | | | | Lotus japonicus | | |
| 15 | 440995 | 440874 | −0.03 | M000065_A149001- | Malate | Organic acids | C₄H₆O₅ | 233 | Tomato, Arabidopsis, | | A |
| | | | | 101_METB_440995_TOF_Malic acid, DL-(3TMS) | | | | | potato, Lotus japonicus | | |
| 16 | 452200 | 452236 | 0.04 | M000114_A153003- | GABA | Amino acids | C₄H₉NO₂ | 174 | Tomato, Arabidopsis, | | A |
| | | | | 101_METB_452200_TOF_Butyric acid, 4-amino-(3TMS) | | | | | potato, Lotus japonicus | | |
| 17 | 457283 | 457208 | 0.1 | M000033_A152002- | Aspartate | Amino acids | C₄H₇NO₄ | 232 | Tomato, Arabidopsis, | | A |
| | | | | 101_METB_457283_TOF_Aspartic acid, L-(3TMS) | | | | | potato | | |
| 18 | 474325 | 474425 | 0 | M000018_A152001- | Methionine | Amino acids | C₅H₁₁NO₂S | 176 | Tomato, Arabidopsis, | | A |
| | | | | 101_METB_474325_TOF_Methionine, DL-(2TMS) | | | | | potato, Lotus japonicus | | |
| 19 | 492650 | 493001 | 0.07 | M000032_A174008- | Glutamine | Amino acids | C₅H₁₀N₂O₃ | 227 | Tomato, Arabidopsis | | A |
| | | | | 101_METB_492650_TOF_Glutamine, DL-(4TMS) | | | | | | | |
| 20 | 498150 | 497738 | −0.09 | M000579_A166001- | Xylose | Sugar (Pentose, | C₅H₁₀O₅ | 103 | Nicotiana tabacum, | | A |
| | | | | 101_METB_498150_TOF_Xylose, D-(1MEOX) (4TMS) | | aldos [symbol] | | | Arabidopsis, Lotus | | |
| | | | | | | | | | japonicus | | |
| 21 | 497920 | 497738 | −0.04 | M000576_A165006- | Lyxose | Sugar (Pentose, | C₅H₁₀O₅ | 103 | Nicotiana tabacum, | | A |
| | | | | 101_METB_497920_TOF_Lyxose, D-(1MEOX) (4TMS) | | aldos [symbol] | | | Arabidopsis, Lotus | | |
| | | | | | | | | | japonicus | | |
| 22 | 531145 | 531384 | 0.04 | M000011_A164001- | Phenylalanine | Amino acids | C₉H₁₁NO₂ | 218 | Tomato, Arabidopsis, | | A |
| | | | | 101_METB_531145_TOF_Phenylalanine, DL-(2TMS) | | | | | potato, Lotus japonicus | | |
| 23 | 550100 | 550158 | 0 | M000013_A168001- | Asparagine | Amino acids | C₄H₈N₂O₃ | 116 | Tomato, Arabidopsis, | | A |
| | | | | 101_METB_550100_TOF_Asperagine, DL-(3TMS) | | | | | potato | | |
| 24 | 553540 | 553211 | −0.06 | M000592_A172006- | 2-amino-Adipic | Amino acids | C₆H₁₁NO₄ | 217 | Tomato, Arabidopsis | | A |
| | | | | 101_METB_553540_TOF_Adipic acid, 2-amino, DL- | acid | | | | | | |
| | | | | (3TMS) | | | | | | | |
| 25 | 557320 | 556810 | −0.06 | M000248_A172001- | 1,6-anhydro, | Sugar | C₆H₁₀O₅ | 204 | Tomato, Arabidopsis | | A |
| | | | | 101_METB_557320_TOF_Glucose, 1,6-anhydro, beta-D- | beta-D-Glucose | (anhydride) | | | | | |
| | | | | (3TMS) | | | | | | | |
| 26 | 570427 | 570652 | 0.55 | M000028_A182002- | Ornithine | Polyamine | C₅H₁₂N₂O₂ | 142 | Tomato, Arabidopsis | | A |
| | | | | 101_METB-METB_570427_TOF_Ornithine, DL-(4TMS) | | | | | | | |
| 27 | 574230 | 574093 | 0.51 | M000328_A177002- | Glycerol-3-P | Triol phosphate | C₃H₉O₆P | 299 | Tomato, Arabidopsis, | | A |
| | | | | 101_METB_574230_TOF_Glycerol-3-phosphate, DL- | | | | | potato | | |
| | | | | (4TMS) | | | | | | | |
| 28 | 579380 | 579208 | 0.94 | M000606_A187002- | Fructose | Sugar (Hexose) | C₆H₁₂O₆ | 217 | Tomato, Arabidopsis, | | A |
| | | | | 101_METB_579380_TOF_Fructose, D-(1MEOX) (5TMS) | | | | | potato, Lotus japonicus | | |
| 29 | 587270 | 587242 | 0 | M000043_A191002- | Galactose | Sugar (Hexose, | C₆H₁₂O₆ | 319 | Tomato, potato, Lotus | | A |
| | | | | 101_METB-METB_587270_TOF_Galactose, D-(1MEOX) | | aldos [symbol] | | | japonicus | | |
| | | | | (5TMS) | | | | | | | |
| 30 | 590400 | 590588 | 0.02 | M000040_A169002- | Glucose | Sugar (Hexose, | C₆H₁₂O₆ | 160 | Tomato, Arabidopsis, | | A |
| | | | | 101_METB_590400_TOF_Glucose, D-(1MEOX) (5TMS) | | aldos [symbol] | | | potato | | |
| 31 | 592883 | 592830 | −0.01 | M000069_A182004- | Citrate | Organic acid | C₆H₈O₇ | 273 | Tomato, Arabidopsis, | | A |
| | | | | 101_METB-METB_592883_TOF_Citric acid (4TMS) | | | | | potato, Lotus japonicus | | |
| 32 | 625030 | 625442 | 0.16 | M000638_A189008- | 1,5-lactone- | Acid | C₆H₁₀O₆ | 129 | Tomato, Arabidopsis | | A |
| | | | | 101_METB_625030_TOF_Gluconic acid-1,5-lactone, D- | Gluconic acid | (polyhydroxy) | | | | | |
| | | | | (4TMS) | | | | | | | |
| 33 | 653910 | 654328 | 0.08 | M000060_A209002- | myo-inositol | Polyol | C₆H₁₄O₆ | 191 | Tomato, Arabidopsis, | | A |
| | | | | 101_METB_653910_TOF_Inositol, myo-(6TMS) | | | | | potato | | |
| 34 | 658337 | 658465 | −0.01 | M000035_A194002- | Tyrosince | Amino acids | C₉H₁₁NO₃ | 218 | Tomato, Arabidopsis, | | A |
| | | | | 101_METB_658337_TOF_Tyrosine, DL-(3TMS) | | | | | potato, Lotus japonicus | | |
| 35 | 790770 | 790644 | −0.03 | M000012_A223002- | Tryptophane | Amino acids | C₁₁H₁₂N₂O₂ | 202 | Tomato, Arabidopsis, | | A |
| | | | | 101_METB_790770_TOF_Trptophan, L-(2TMS) | | | | | potato | | |
| 36 | 876240 | 876596 | 0.04 | M000671_A274002- | Trehalose | Sugar | C₁₂H₂₂O₁₁ | 169 | Tomato, Arabidopsis, | | A |
| | | | | 101_METB_876240_TOF_Trehalose, alpha,alpha′-, D | | (disaccharide) | | | potato | | |
| | | | | (8TMS) | | | | | | | |
| 37 | 887905 | 888922 | 0.07 | M000048_A277002- | Maltose | Sugar | C₁₂H₂₂O₁₁ | 361 | Tomato, Arabidopsis, | | A |
| | | | | 101_METB_887505_TOF_Maltose, D-(1MEOX) (8TMS) | | (disaccharide) | | | potato, Lotus japonicus | | |
| 38 | 940280 | 940132 | −0.02 | M000673_A299002- | Galactinol | Conjugate | C₁₂H₂₂O₁₁ | 204 | Tomato, Arabidopsis, | | A |
| | | | | 101_METB_940280_EIROE_Galactinol (9TMS) | | (Hexosyl, | | | potato, Lotus japonicus | | |
| | | | | | | In [symbol] | | | | | |
| 39 | 1033367 | 1033424 | 0 | M000049_A337002- | Raffinose | Sugar | C₁₈H₃₂O₁₆ | 217 | Tomato, Arabidopsis, | | A |
| | | | | 101_METB_1033367_TOF_Raffinose (11TMS) | | (trisaccharide) | | | potato, Lotus japonicus | | |
| 40 | 507780 | 507895 | 0.01 | M000036_A163001- | Glutamate | Amino acids | C₅H₈NO₅ | 246 | Tomato, Arabidopsis, | | A |
| | | | | 101_METB_507760_TOF_Glutamic acid, DL-(3TMS) | | | | | potato, Lotus japonicus | | |
| 41 | 626910 | 627066 | 0.03 | M000508_A200001- | Gluconic acid | Acid | C₆H₁₂O₇ | 292 | Tomato, Arabidopsis | | A |
| | | | | 101_METB_626910_TOF_Gluconic acid (6TMS) | | (polyhydroxy) | | | | | |
| 42 | 840783 | 841382 | 0.06 | M000044_A264001- | Sucrose | Sugar | C₁₂H₂₂O₁₁ | 361 | Tomato, Arabidopsis, | | A |
| | | | | 101_METB_840783_TOF_Sucrose, D-(8TMS) | | (disaccharide) | | | potato, Lotus japonicus | | |

Supplemental file 1. Overview of the metabolite reporting list.
GC-TOF-MS metabolites

| | |
|---|---|
| Experiment title: | Metabolite profiles of Arabidopsis dry seeds of WT (Col-0), frk6 (725), frk7 (463) and the double mutant. |
| Organism/Plant species: | Arabidopsis thaliana |
| Organ/tissue: | Dry seed |
| Analytical tool: | GC-TOF-MS |

Peak/compound no.—number referenced back to the main text
Ret. Time—Time expected, Tag Time Index and Time deviation
Putative Name—putative identification of the metabolite/derivative
Corresponding metabolite name in literature
Mol. Formula—molecular formula of the metabolite or its FA adduct;
Mass to charge ratio (m/z)
(S)-identification confirmed by a standard compound
I, II, III—different isomers
Identification level (A; B; C; D)-(A) standard or NMR; (B) MS/MS; (C) MS^{E}; (D) MS only

| | | | | | | | | Mass to | | | |
|---|---|---|---|---|---|---|---|---|---|---|---|
| Peak/ | | Tag | Time | | Corresponding | | | charge | | | fication |
| Compound | Time | Time | Devi- | | Metabolite | Metabolite | | ratio | Species | Refer- | level |
| no. | Expected | Index | ation | Putative metabolite name (Derivative) | in Literature | Class | Mol formula | (m/z) | detected before | ences | (A-D) |
| 1 | 208565 | 209107 | 0.29 | M000026_A110001- | Alanine | Amino acids | C₃H₇NO | 116 | Tomato, Arabidopsis, | | A |
| | | | | 101_METB_208565_TOF_Alanine, DL-(2TMS) | | | | | potato, Lotus japonicus | | |
| 2 | 271580 | 271627 | −0.03 | M000030_A122001- | Valine | Amino acids | C₅H₁₁NO [symbol] | 144 | Tomato, Arabidopsis, | | A |
| | | | | 101_METB_271580_TOF_Valine, DL-(2TMS) | | | | | potato, Lotus japonicus | | |
| 3 | 291783 | 291982 | 0.08 | M000053_A129003- | lycerol | Polyol (Triol) | C3H8O3 | 117 | Tomato, Arabidopsis, | | A |
| | | | | 101_METB_291783_TOF_Glycerol (3TMS) | | | | | potato, Lotus japonicus | | |
| 4 | 305020 | 305084 | 0.04 | M000025_A129002- | Leucine | Acid (Amino) | C6H13NO [symbol] | 158 | Tomato, Arabidopsis, | | A |
| | | | | 101_METB_301020_TOF_Leucine, DL-(2TMS) | | | | | potato, Lotus japonicus | | |
| 5 | 319193 | 319027 | 0.03 | M000017_A132002- | [symbol] soleucine | Amino acids | C₆H₁₃NO [symbol] | 158 | Tomato, Arabidopsis, | | A |
| | | | | 101_METB_319193_TOF_isoleucine, L-(2TMS) | | | | | potato | | |
| 6 | 333520 | 332772 | −0.21 | M000075_A129001- | Phosphase | Inorganic acid | H3O4P | 299 | Tomato, Arabidopsis, | | A |
| | | | | 101_METB_333520_TOF_Phosphoric acid (3TMS) | | | | | potato, Lotus japonicus | | |
| 7 | 338693 | 338504 | −0.05 | M000029_A132003- | Proline | Amino acids | C₅H₉NO₂ | 142 | Tomato, Arabidopsis, | | A |
| | | | | 101_METB_338693_TOF_Proline, L-(2TMS) | | | | | potato | | |
| 8 | 357523 | 357478 | 0.02 | M000015_A138001- | Serine | Amino acids | C₃H₇NO₃ | 204 | Tomato, Arabidopsis, | | A |
| | | | | 101_METB_357523_TOF_Serine, DL-(2TMS) | | | | | potato | | |
| 9 | 365427 | 365280 | −0.03 | M000074_A134001- | Succinate | Organic acids | C₄H₆O₄ | 129 | Tomato, Arabidopsis, | | A |
| | | | | 101_METB_365427_TOF_Succinic acid (2TMS) | | | | | potato, Lotus japonicus | | |
| 10 | 368365 | 367456 | −0.23 | M000016_A140001- | Threonine | Amino acids | C₄H₈NO₃ | 117 | Tomato, Arabidopsis, | | A |
| | | | | 101_METB_368365_TOF_Threonine, DL-(3TMS) | | | | | potato | | |
| 11 | 371255 | 371138 | −0.03 | M000067_A137001- | Fumarate | Organic acid | C₄H₄O₄ | 245 | Tomato, Arabidopsis, | | A |
| | | | | 101_METB_371255_TOF_Fumaric acid (2TMS) | | | | | potato, Lotus japonicus | | |
| 12 | 392490 | 391823 | −0.17 | M000456_A136001- | Uracil | Pyrimidine | C4H4N2O [symbol] | 99 | Tomato, potato, Lotus | | A |
| | | | | 101_METB_392490_TOF_Uracil (2TMS) | | | | | japonicus | | |
| 13 | 394250 | 394080 | −0.04 | M000027_A144001- | beta-alanine | Amino acids | C₃H₇NO | 174 | Tomato, Arabidopsis, | | A |
| | | | | 101_METB_394250_TOF_Alanine, beta-(3TMS) | | | | | potato, Lotus japonicus | | |
| 14 | 440995 | 441209 | 0.06 | M000065_A149001- | Malate | Organic acids | C₄H₆O₅ | 233 | Tomato, Arabidopsis, | | A |
| | | | | 101_METB_440995_TOF_Malic acid, DL-(3TMS) | | | | | potato, Lotus japonicus | | |
| 15 | 452200 | 452226 | 0.03 | M000114_A153003- | GABA | Amino acids | C₄H₉NO₂ | 174 | Tomato, Arabidopsis, | | A |
| | | | | 101_METB_452200_TOF_Butyric acid, 4-amino-(3TMS) | | | | | potato, Lotus japonicus | | |
| 16 | 457283 | 457362 | 0.06 | M000033_A152002- | Aspartate | Amino acids | C₄H₇NO₄ | 232 | Tomato, Arabidopsis, | | A |
| | | | | 101_METB_457283_TOF_Aspartic acid, L-(3TMS) | | | | | potato | | |
| 17 | 474325 | 474243 | −0.01 | M000018_A152001- | Methionine | Acid (Amino) | C5H11NO [symbol] | 176 | Tomato, Arabidopsis, | | A |
| | | | | 101_METB_474325_TOF_Methionine, DL-(2TMS) | | | | | potato, Lotus japonicus | | |
| 18 | 506483 | 506504 | 0.01 | M000037_A153002- | Pyroglutamate | Lactam | C5H7NO3 | 156 | Tomato, Arabidopsis, | | A |
| | | | | 101_METB-METB_506483_TOF_Pyroglutamic acid, DL- | | | | | potato | | |
| | | | | (2TMS [symbol] | | | | | | | |
| 19 | 507780 | 507982 | 0.07 | M000036_A163001- | Glutamate | Amino acids | C₅H₉NO₅ | 246 | Tomato, Arabidopsis, | | A |
| | | | | 101_METB_507780_TOF_Glutamic acid, DL-(3TMS) | | | | | potato, Lotus japonicus | | |
| 20 | 517180 | 517180 | 0 | M000196_A175002- | Putrescine | Polyamine | C₄H₁₂O₂ | 174 | Tomato, Arabidopsis, | | A |
| | | | | 101_METB-METB_517180_TOF_Putrescine (4TMS) | | | | | potato, Lotus japonicus | | |
| 21 | 531145 | 530926 | −0.08 | M000011_A164001- | Phenylalanine | Amino acids | C₉H₁₁NO [symbol] | 218 | Tomato, Arabidopsis, | | A |
| | | | | 101_METB_531145_TOF_Phenylalaline, DL-(2TMS) | | | | | potato, Lotus japonicus | | |
| 22 | 550100 | 550159 | 0.02 | M000013_A168001- | Asparagine | Amino acids | C₄H₈N₂O [symbol] | 116 | Tomato, Arabidopsis, | | A |
| | | | | 101_METB_550100_TOF_Asparagine, DL-(3TMS) | | | | | potato | | |
| 23 | 574230 | 574188 | −0.01 | M000328_A177002- | Glycerol-3-P | Triol phosphate | C3H9O6P [symbol] | 299 | Tomato, Arabidopsis, | | A |
| | | | | 101_METB_574230_TOF_Glycerol-3-phosphate, DL- | | | | | potato | | |
| | | | | (4TMS) | | | | | | | |
| 24 | 577930 | 577852 | −0.01 | M000007_A185001- | Quinate | Acid (Hydroxy) | C7H12O [symbol] | 345 | Tomato, potato, Lotus | | A |
| | | | | 101_METB_577930_TOF_Quinic acid, D(-)-(5TMS) | | | | | japonicus | | |
| 25 | 579380 | 579313 | 0.03 | M000606_A188004- | Fructose | Sugar (Hexose) | C6H12O [symbol] | 217 | Tomato, Arabidopsis, | | A |
| | | | | 101_xxx_NA_642058.88_TRUE_VAR5_FAME_Fructose | | | | | potato, Lotus japonicus | | |
| | | | | (1MEOX [symbol] | | | | | | | |
| 26 | 587270 | 587245 | 0.03 | M000043_A188001- | Galactose | Sugar (Hexose, | C6H12O [symbol] | 319 | Tomato, potato, Lotus | | A |
| | | | | 101_METB_586000_TOF_Galactose, D-(1MEOX) | | [symbol] | | | japonicus | | |
| | | | | (5TMS) | | | | | | | |
| 27 | 590400 | 590376 | 0.07 | M000040_A180002- | Glucose | Sugar (Hexose, | C6H12O [symbol] | 160 | Tomato, Arabidopsis, | | A |
| | | | | 101_METB_590400_TOF_Glucose, D-(1MEOX) (5TMS) | | [symbol] | | | potato | | |
| 28 | 592883 | 592675 | −0.02 | M000069_A182004- | Citrate | Organic acid | C₆H₈O₇ | 273 | Tomato, Arabidopsis, | | A |
| | | | | 101_METB-METB_592883_TOF_Citric acid (4TMS) | | | | | potato, Lotus japonicus | | |
| 29 | 615467 | 615181 | −0.02 | M000014_A192003- | Lysine | Amino acids | C₆H₁₄N₂O [symbol] | 156 | Tomato, Arabidopsis, | | A |
| | | | | 101_METB_615467_TOF_Lysine, L-(4TMS) | | | | | potato | | |
| 30 | 624990 | 624858 | 0.05 | M000082_A185002- | Dehydroascorbate | Hydroxy acid | C₆H₆O₆ | 173 | Tomato, Arabidopsis, | | A |
| | | | | 101_METB-METB_624990_TOF_Dehydroascorbic acid | | | | | potato | | |
| | | | | dimer ( [symbol] | | | | | | | |
| 31 | 638360 | 638235 | −0.02 | M000081_A191004- | Tyramine | Amine | C8H11NO [symbol] | 174 | Tomato, Arabidopsis, | | A |
| | | | | 101_METB_638360_TOF_Tyramine (3TMS) | | (Aromatic) | | | potato | | |
| 32 | 653910 | 654040 | 0.04 | M000060_A209002- | myo-inositol | Polyol | C₆H₁₄O₆ | 191 | Tomato, Arabidopsis, | | A |
| | | | | 101_METB_653910_TOF_Inositol, myo-(6TMS) | | | | | potato | | |
| 33 | 658337 | 658129 | −0.02 | M000035_A194002- | Tyrosine | Amino acids | C₉H₁₁NO [symbol] | 218 | Tomato, Arabidopsis, | | A |
| | | | | 101_METB_658337_TOF_Tyrosine, DL-(3TMS) | | | | | potato, Lotus japonicus | | |
| 34 | 679450 | 678360 | −0.17 | M000039_A192006- | Histidine | Amino acids | C₈H₉N₃O [symbol] | 154 | Tomato, Arabidopsis, | | A |
| | | | | 101_METB_679450_TOF_Histidine, L-(3TMS) | | | | | potato | | |
| 35 | 790560 | 790178 | −0.02 | M000012_A223001- | Tryptophane | Amino acids | C₁₁H₁₂N₂ | 202 | Tomato, Arabidopsis, | | A |
| | | | | 101_METB_790560_TOF_Tryptophan, L-(3TMS) | | | | | potato | | |
| 36 | 840783 | 841657 | 0.13 | M000044_A264001- | Sucrose | Sugar | C₁₂H₂₂O₁ [symbol] | 451 | Tomato, Arabidopsis, | | A |
| | | | | 101_METB_840783_TOF_Sucrose, D-(8TMS) | | (disaccharide) | | | potato, Lotus japonicus | | |
| 37 | 870355 | 869731 | −0.07 | M000048_A274001- | Maltose | Sugar | C₁₂H₂₂O₁ [symbol] | 204 | Tomato, Arabidopsis, | | A |
| | | | | 101_METB_870355_TOF_Maltose, D-(1MEOX) (8TMS) | | (disaccharide) | | | potato, Lotus japonicus | | |
| 38 | 876240 | 875660 | −0.06 | M000671_A274002- | Trehalose | Sugar | C₁₂H₂₂O₁ [symbol] | 191 | Tomato, Arabidopsis, | | A |
| | | | | 101_METB_876240_TOF_Trehalose, alpha,alpha′-, D- | | (disaccharide) | | | potato | | |
| | | | | (8TMS) | | | | | | | |
| 39 | 917000 | 916604 | −0.04 | M000107_A287001- | Isomaltose | Sugar | C12H22O1 [symbol] | 160 | Tomato, potato | | A |
| | | | | 101_METB_909948_TOF_Isomaltose (1MEOX) (8TMS) | | (Disaccharide) | | | | | |
| 40 | 940280 | 939261 | −0.11 | M000673_A299002- | [symbol] alactinol | Conjugate | C12H22O [symbol] | 204 | Tomato, Arabidopsis, | | A |
| | | | | 101_METB_940280_EIROE_Galactinol (9TMS) | | (Hexosyl, | | | potato, Lotus japonicus | | |
| | | | | | | [symbol] nositol) | | | | | |
| 41 | 1033367 | 10327396 | −0.04 | M000049_A337002- | Raffinose | Sugar | C₁₈H₃₂O₁ [symbol] | 217 | Tomato, Arabidopsis, | | A |
| | | | | 101_METB_1033367_TOF_Raffinose (11TMS) | | (trisaccharide) | | | potato, Lotus japonicus | | |

[symbol]

indicates data missing or illegible when filed

Fatty Acid (FAMEs) Extraction and Measurement Using Gas Chromatography Coupled to a Flame Ionization Detector (GC-FID)

For each line, 20 mature Arabidopsis seeds from six individual plants were counted and carefully weighed using analytical scales. Seed fatty acids were extracted exactly as described previously (Focks & Benning, 1998). Briefly, seeds were homogenized and incubated with 1 ml 1 N HCl in methanol. To each sample, 100 μl of internal standard (FA15:0, pentadecanoic acid) were added. Samples were then incubated at 80° C. in a water bath for 30 min. After the vials had cooled down to the room temperature, 1 ml of 0.9% NaCl and 1 ml 100% hexane were added to each vial. Vials were shaken for 5 s and centrifuged for 4 min at 1000 rpm. The upper FAMEs-containing hexane phase was transferred to a new glass vial, where it was concentrated in an N₂ stream. Finally, FAMEs were dissolved in hexane and poured into GC glass vials. The GC-FID method data: injector temperature: 250° C.; carrier gas: helium; head pressure: 25 cm sec⁻¹, 11.8 psi; GC-column J&W DB23 (Agilent), 30 m×0.25 mm×0.25 μ.m; detector: 250° C.; detector gas: H₂ 40 ml min⁻¹, air 450 ml min⁻¹, He make-up gas 30 ml min⁻¹.

Anatomical Techniques

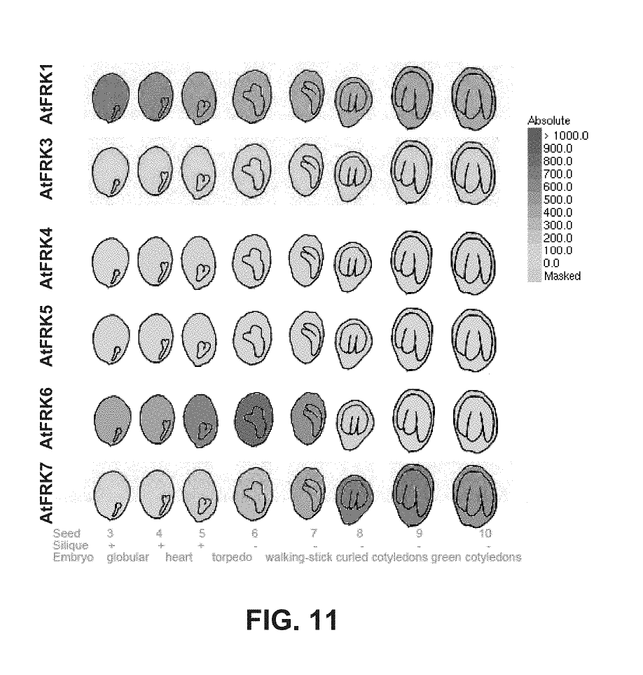

Free-hand cross-sections were taken from 8-week-old WT, quadruple-mutant and penta-mutant plants as shown in FIG. 11. The cross-sections were stained for a few seconds in 2% lacmoid in 96% ethanol and then rinsed in tap water for a few minutes, mounted in 50% sodium lactate (Aloni, 1980) and observed under transmitted white light.

Transmission Electron Microscopy (TEM)

Mature, dry Arabidopsis Col-0 and frk6 frk7 seeds were placed on adhesive tape and a small portion of the seed was cut out with a sharp knife, in order to allow processing material easy access to the embryo. The seeds were fixed in 5% glutaraldehyde in 0.1 M cacodylate buffer (pH 7.4) overnight at room temperature. The tissues were then rinsed four times, 10 min each rinse, in cacodylate buffer and post-fixed and stained with 2% osmium tetroxide, 1.5% potassium ferricyanide in 0.1 M cacodylate buffer for 1 hour. Tissues were then washed four times in cacodylate buffer and dehydrated in increasing concentrations of ethanol (i.e., 30%, 50%, 70%, 80%, 90%, 95%),10 min each step, followed by three 20-min dehydrations in 100% anhydrous ethanol and two 10-min dehydrations in propylene oxide. Following dehydration, the tissues were infiltrated with increasing concentrations of Agar 100 resin (i.e., 25, 50, 75, and 100%) in propylene oxide, with each step lasing 16 h. The tissues were then embedded in fresh resin and left to polymerize in an oven at 60° C. for 48 h.

Embedded tissues in blocks were sectioned first with a glass knife (2 μm) and stained with methylene blue, and then with a diamond knife on an LKB 3 microtome and ultrathin sections (80 nm) were collected onto 200-mesh, carbon/formvar-coated copper grids. The sections on grids were sequentially stained with uranyl acetate and lead citrate for 10 min each and viewed with Tecnai 12 TEM 100 kV (Phillips, Eindhoven, the Netherlands) equipped with MegaView II CCD camera and Analysis software, version 3.0 (Softlmaging System GmbH, Munstar, Germany).

### Example 1

### Identification of Arabidopsis Frk Genes

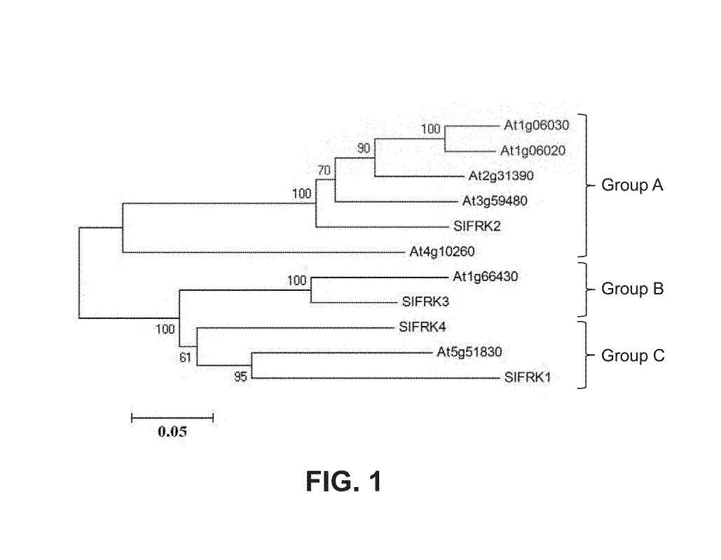

Protein blast analysis (http://blast(dot)ncbi(dot)nlm(dot)nih(dot)gov/Blast(dot)cgi) with the four characterized tomato FRK proteins against Arabidopsis proteins identified seven genes from the pfkb protein family denoted as probable FRK1-7 by the uniprotKB/swissprot plant proteome annotation program (Schneider et al., 2009). A phylogenetic tree created from Arabidopsis and tomato FRKs proteins (FIG. 1) showed that one protein (At1g66430, assigned FRK6) is a homolog of the plastidic SlFRK3 and that another, Arabidopsis FRK (At5g51830, assigned FRK7), is closely related to SIFRK1, while five other proteins (At2g31390, At1g06030, At1g06020, At3g59480, At4g10260 assigned FRK1-5, respectively) are closely related to SlFRK2, the main cytosolic FRK in tomato. It appears that AtFRK5 might be functionally related to SlFRK4 as both are expressed exclusively in pollen (David-Schwartz et al., 2013). Prediction of the subcellular localization of the Arabidopsis fructokinases suggests that AtFRK6 is the only plastidic protein; the others are most likely cytosolic (Wolf Psort—http://www(dot)genscript(dot)com/wolf-psort(dot)html, TargetP—http://www(dot)cbs(dot)dtu(dot)dk/services/TargetP/) (FIGs . 2A-B). This prediction is in line with mass spectrometry data showing that AtFRK1 and AtFRK7 are present in the cytosol (Ito et al., 2011), while AtFRK6 is present in the chloroplasts and, more specifically, in the stroma (Peltier et al., 2006; Rutschow et al., 2008; Zybailov et al., 2008; Ferro et al., 2010; Olinares et al., 2010; Helm et al., 2014).

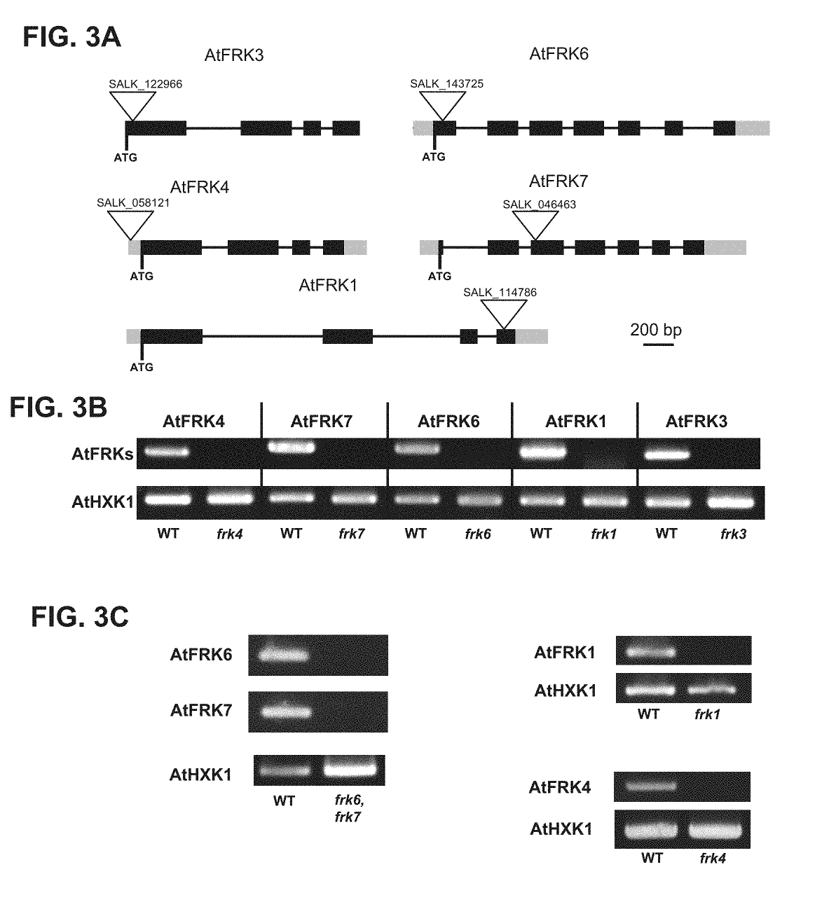

In order to characterize the function of AtFRKs in planta, T-DNA mutants of five of the AtFRK genes (AtFRK1,3,4,6,7) were identified and obtained from ABRC stocks (https://abrc(dot)osu(dot)edu). The T-DNA Salk mutants are referred as frkl, frk3, frk4, frk6 and frk7 (Table 2). The T-DNA Salk lines were characterized and confirmed as containing the T-DNA insertion in the corresponding genes based on PCR with primers from both sides of the T-DNA insertion (Table 2) and the absence of the related FRK mRNA (FIGS. 3A-C).

### Example 2

### The Arabidopsis Double-Mutant (FRK6, FRK7) Exhibits A Specific Seed Phenotype

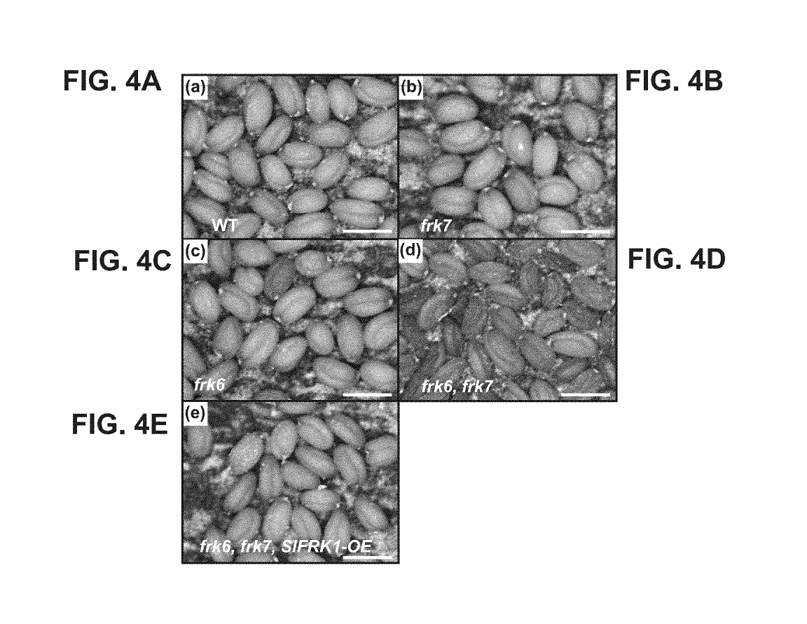

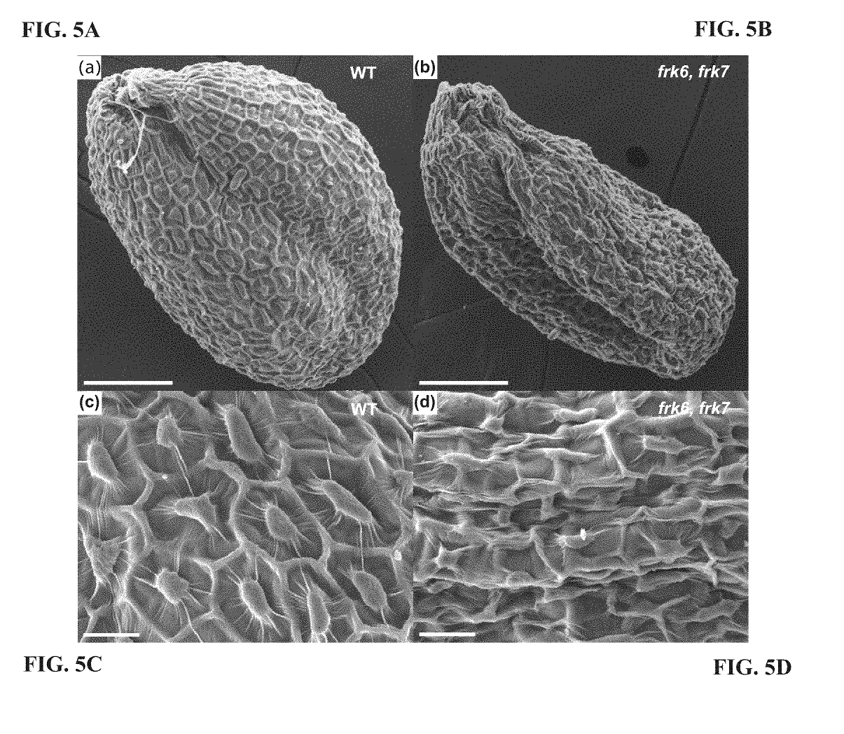

Neither one of the single T-DNA mutants of the Arabidopsis FRKs showed any visibly unusual phenotype when grown under normal growth conditions in soil or plates. Therefore, crosses were made between the individual lines to obtain homozygous double and multiple mutants, assuming that the AtFRKs' functions might be redundant. We obtained most of the double-mutant combinations and also created triple-, quadruple- and penta-mutants for different T-DNA lines. All of the double-mutants exhibited normal growth and produced normal seeds, except for a single double-mutant composed of the plastidic AtFRK6 (frk6) and the cytosolic AtFRK7 (frk7) whose seeds exhibited a unique phenotype. Those seeds were thin and dark and weighed 13% less than WT seeds (FIGS. 4A, D Table 2). Scanning electron microscope images showed that seeds of frk6 frk7 double-mutants were wrinkled at various degrees and had an abnormal surface (FIGS. 5A-D, 6A-G).

### Example 3

### frk6 frk7 Double-Mutant Seedlings Exhibit Arrested Growth That Can be Overcome by the Addition of External Sugar or the Expression of Tomato SlFRK1

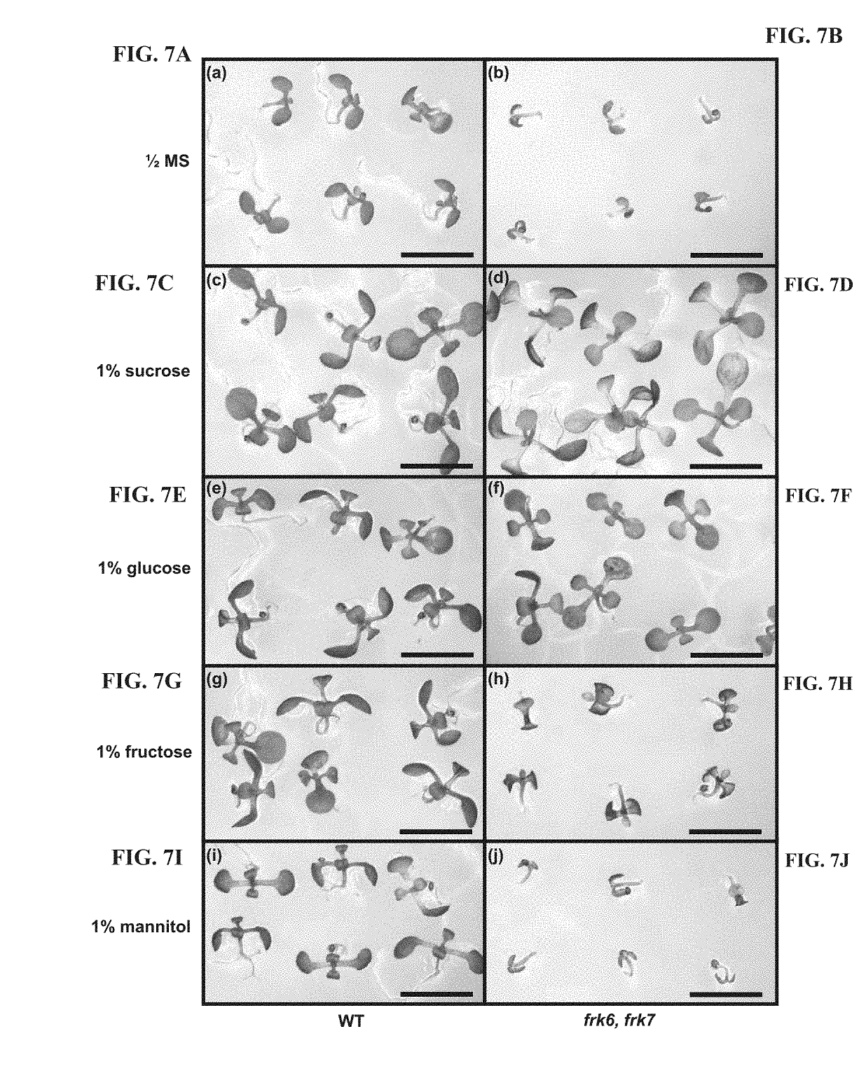

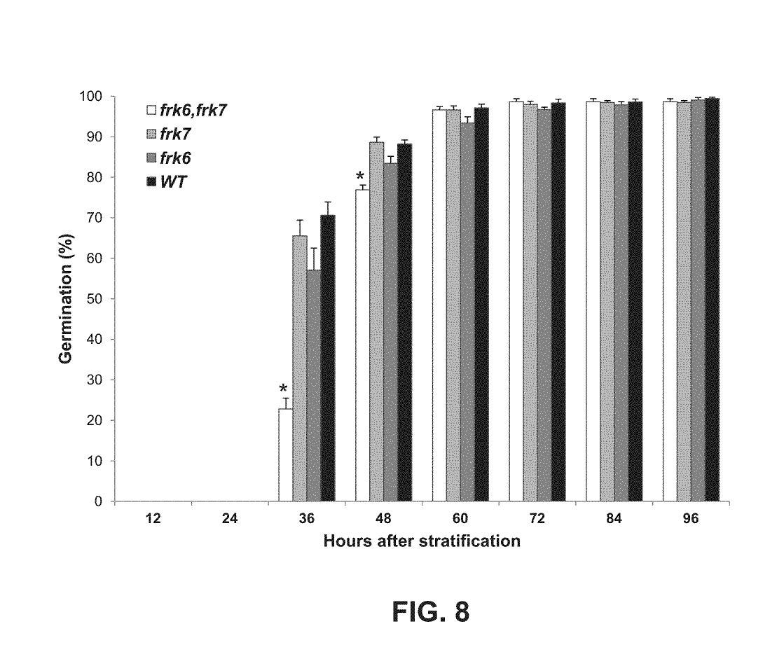

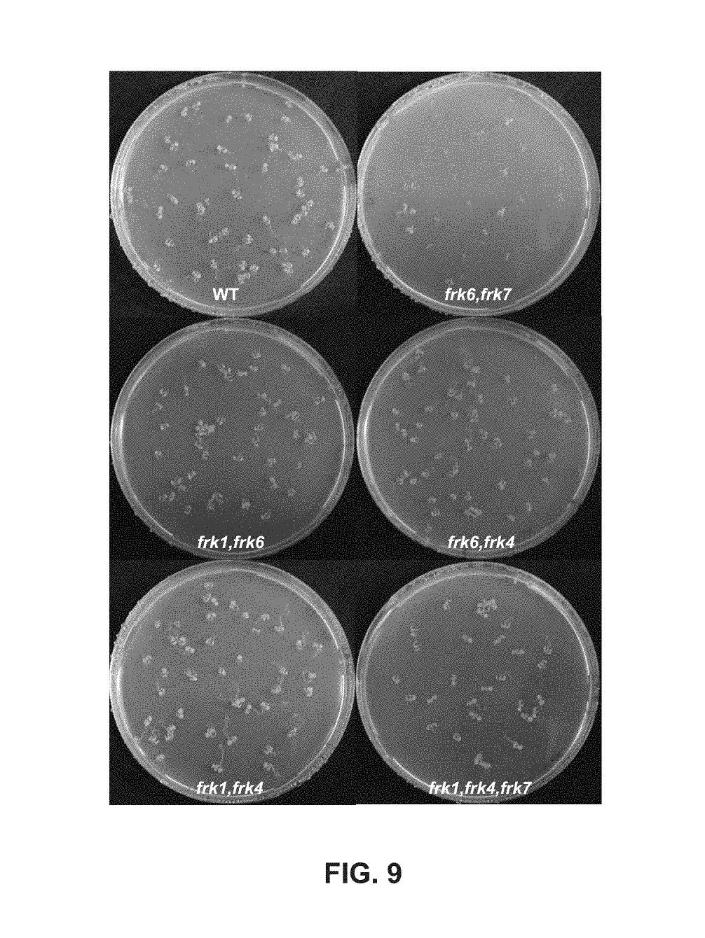

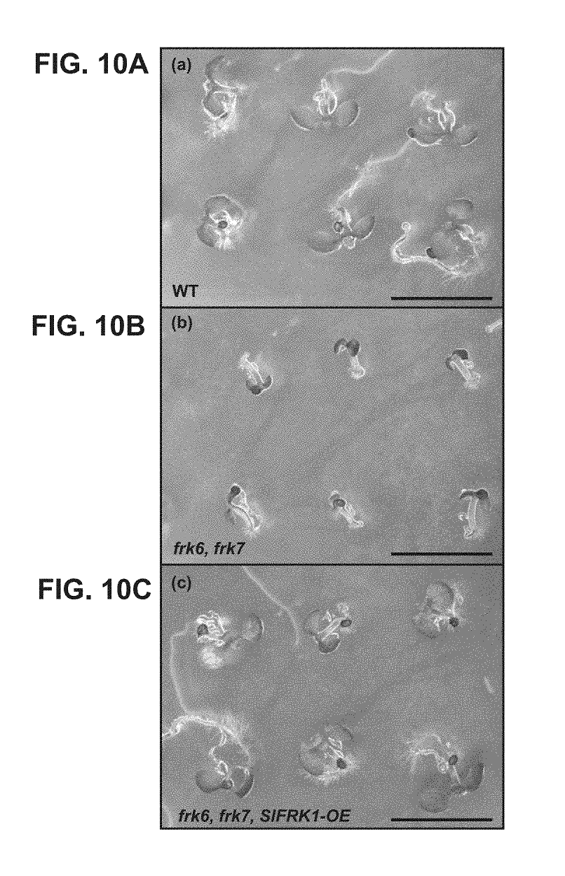

The abnormal shape and low weight of the double-mutant seeds raised the possibility that the germination and seedling development of these seeds might be affected. However, no growth defects were observed when the seeds of the double-mutant were sown in soil. When the seeds were grown on ½ MS plates with no added sugar, the double-mutant seeds germinated, but stopped growing soon after germination (radicle and cotyledon emergence) and were unable to develop true leaves (FIGS. 7A-B). The germination percentage was not different from that of the WT seeds, but the germination rate was slightly slower (FIG. 8). Neither of the other double-mutant combinations exhibited such seed germination and growth defects (FIG. 9). The addition of 1% glucose or sucrose to the ½ MS plates overcame the growth arrest of the frk6 frk7 double-mutant. This indicates that the growth arrest was a result of carbon starvation, perhaps due to insufficient accumulation of storage reserves (FIGS. 7C-F). To test whether reduced fructokinase activity is indeed the primary cause of the seeds' wrinkled appearance and the arrested seedling growth, the double-mutant was crossed with an Arabidopsis line overexpressing tomato fructokinase 1 (SlFRK1). Over-expression of SlFRK1 against the background of the double-mutant restored both seed shape (FIG. 4E) and seedling growth on ½ MS plates in the absence of sugar (FIGS. 10A-C), indicating that the double-mutant phenotype was indeed a result of a reduced ability to phosphorylate fructose.

### Example 4

### ATFRK6 and ATFRK7 Are the Main FRKS Expressed in Embryos During Seed Development

To better understand how both the plastidic AtFRK6 and the cytosolic AtFRK7 might affect seed development, AtFRK expression patterns were examined during seed development. Expression data from the mid-globular embryo stage to the green cotyledon stage of seed development were obtained from the microarray experiments that were used to create the gene-expression map of Arabidopsis development (Schmid et al., 2005). It appears that the main AtFRKs expressed in developing seeds are AtFRK1, AtFRK6 and AtFRK7 (FIG. 11). AtFRK1 is expressed throughout seed development with the highest expression levels reached at the early stages of seed development (FIGS. 7A-J). AtFRK6 is mainly expressed at earlier stages of seed development with the highest expression level observed around the torpedo stage; whereas AtFRK7 is mainly expressed in the later stages of development, with its highest expression level seen around the curled-cotyledon stage (FIG. 11).

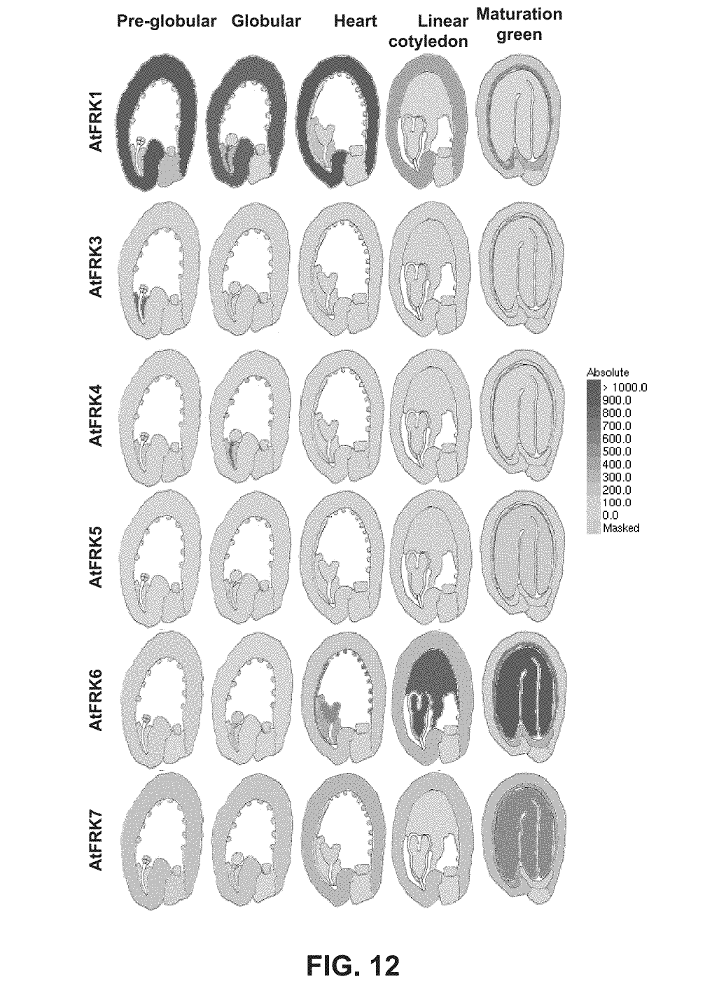

To explore in which seed compartment the different AtFRKs are expressed, the expression data obtained from work done to identify seed-specific transcription factors was analyzed. That work involved laser-capture microdissection of the different seed compartments during different stages of embryo development (Le et al., 2010). AtFRK6 is expressed primarily in the embryo and the endosperm beyond the embryo heart stage with highest expression level seen at the mature green embryo stage (FIG. 12). AtFRK7 is expressed in the seed coat throughout seed development, but is also highly expressed in the embryo during the mature green stages (FIG. 12). Except for these two FRK genes, only AtFRK5 is expressed in the embryo at a low level at the mature green stage. The other FRK genes are expressed in seed tissues other than the embryo (FIG. 12). AtFRK1 is mainly expressed in the seed coat and its expression declines during seed development and increases in the peripheral endosperm during the mature green cotyledon stage (FIG. 12). AtFRK3 is expressed primarily in the micropylar endosperm at the pre-globular stage; whereas AtFRK4 is mainly expressed in the suspensor in the globular stage (FIG. 12). It appears that AtFRK6 and AtFRK7 are the main FRKs expressed in the embryo during progressive stages of seed development, which suggests that the phenotype of the frk6 frk7 double-mutant seeds may stem from decreased fructose phosphorylation in the embryo around the mature green stage. The mature green stage is characterized by cell expansion and the accumulation of storage reserves and it is, therefore, likely that AtFRK6 and AtFRK7 supply fructose 6-phosphate (F6P) for the synthesis of storage reserves.

### Example 5

### The Double-Mutant Seeds Show Altered Fatty Acid Metabolism

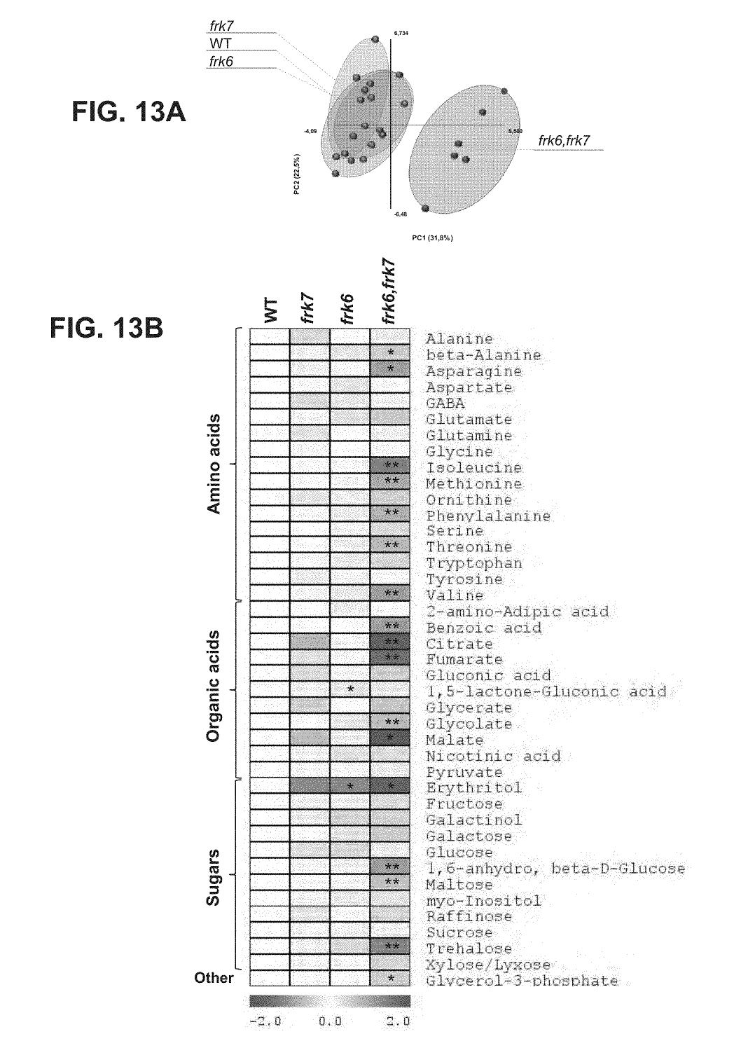

To examine the importance of AtFRK6 and AtFRK7 for the accumulation of seed storage reserves, GC-MS was used to analyze the metabolic profiles of dry seeds of the frk6 frk7 double-mutant, as well as of the single-mutants frk6 and frk7. While there were only slight metabolite changes in each of the single-mutants, there was a major shift in metabolites in the double-mutant seeds (FIG. 13A). The double-mutant seeds contained decreased levels of organic acids, such as citrate, fumarate, malate and benzoic acid, as well as increased levels of glycolate and glycerol 3-phosphate (FIG. 13B). The decreased levels of TCA cycle organic acids (citrate, fumarate and malate) suggest that the F6P that originates from fructose phosphorylated by AtFRK6 and AtFRK7 might be important for the glycolysis that feeds the TCA cycle and that glycolysis in the developing embryos of the double-mutant might be reduced.

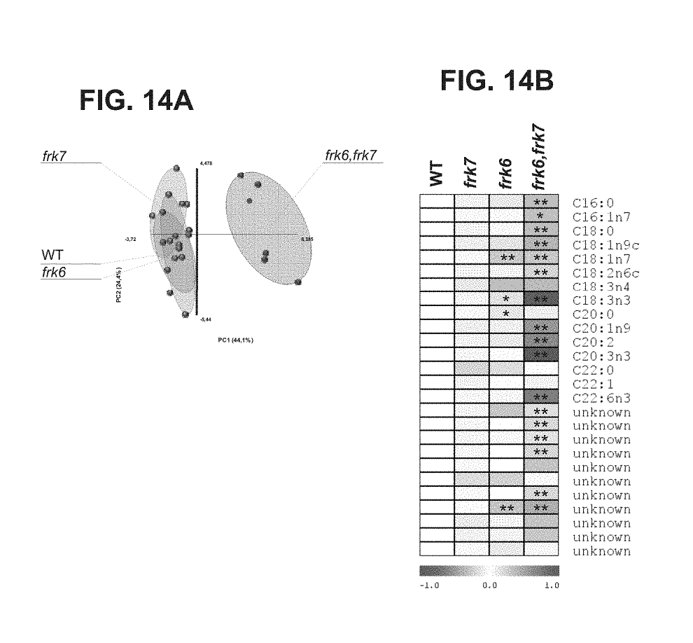

Glycolysis in maturing Arabidopsis seeds is highly important for plastidic acetyl-CoA formation for fatty acid metabolism and oil accumulation (Andre et al., 2007; Baud et al., 2007b). The wrinkled phenotype of the double-mutant seeds is similar to that of Arabidopsis WRINKLED1 mutants whose seeds accumulate less oil (Focks & Benning, 1998; Baud et al., 2007a). This similarity raises the possibility that the frk6 frk7 double-mutant might have reduced fatty acid content. To examine that possibility, seed fatty acid profiling was performed. The fatty acid profiles of frk6 and frk7 single-mutants were similar to that of WT (FIG. 14A) with only small changes in frk6 fatty acids. However, there was a major shift in the fatty acid content of the frk6 frk7 double-mutant (FIG. 14A). The double-mutant showed significantly lower levels of many fatty acids, including palmitic acid (C16:0), stearic acid (C18:0), oleic acid (C18:1n9), linoleic acid (C18:2n6), alpha linolenic acid (C18:3n3), eicosenoic acid (C20:1n9), eicosadienoic acid (C20:2) and eicosatrienoic acid (C20:3n3), which were accompanied by increased levels of palmitoleic acid (C16:1n7), vaccenic acid (C18:1n7), docosahexaenoic acid (C22:6n3) and most of the unknown fatty acids (FIG. 14B). The altered levels of fatty acids and, in particular, the decreases in C16:0, C18:0 and C18:1 suggest that fatty acid synthesis in plastids might be the main metabolic pathway affected by the combined loss of the cytosolic AtFRK6 and the plastidic AtFRK7.

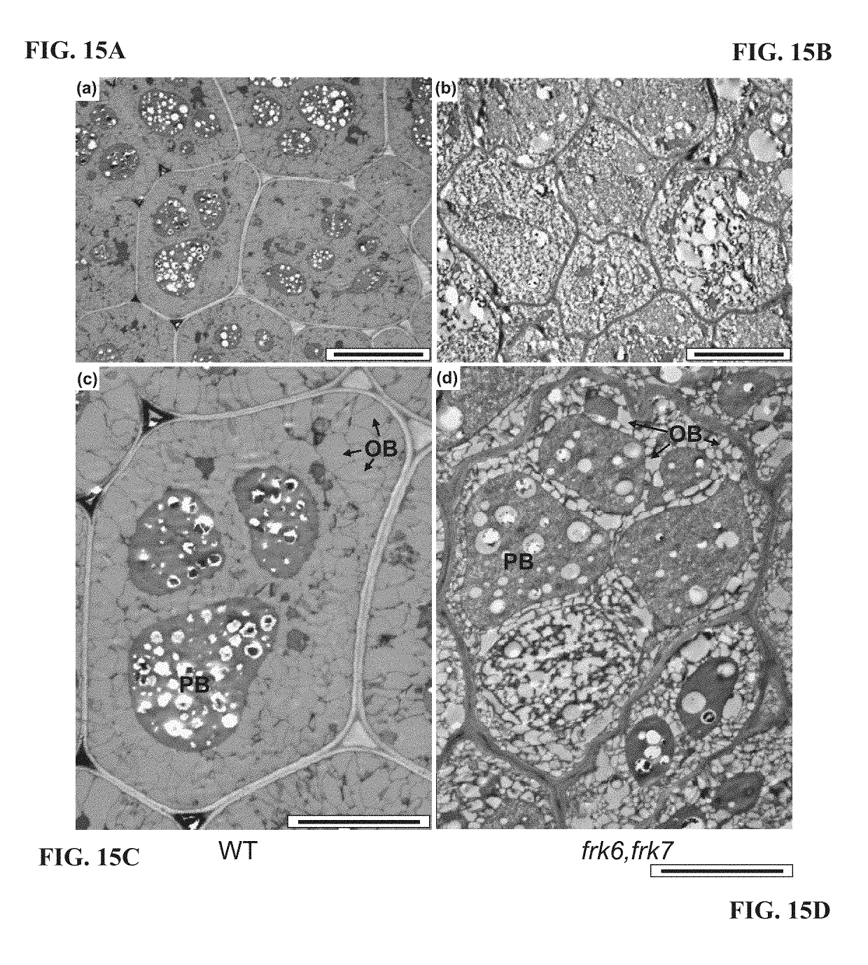

In order to check whether reduced fatty acid synthesis in the double-mutant led to reduced oil accumulation, the embryonic cells of dry seeds were examined using transmission electron microscopy. A decrease in the area and volume of oil bodies (OB) was observed in the double-mutant (FIG. 15D) relative to the WT (FIG. 15B), implying decreased oil content.

### Example 6

### The Arabidopsis Quadruple-Mutant (FRK1, FRK4, FRK6, FRK7) and Penta-Mutant (FRK1, FRK3, FRK4, FRK6, FRK7) Show Altered Vascular Tissue Development

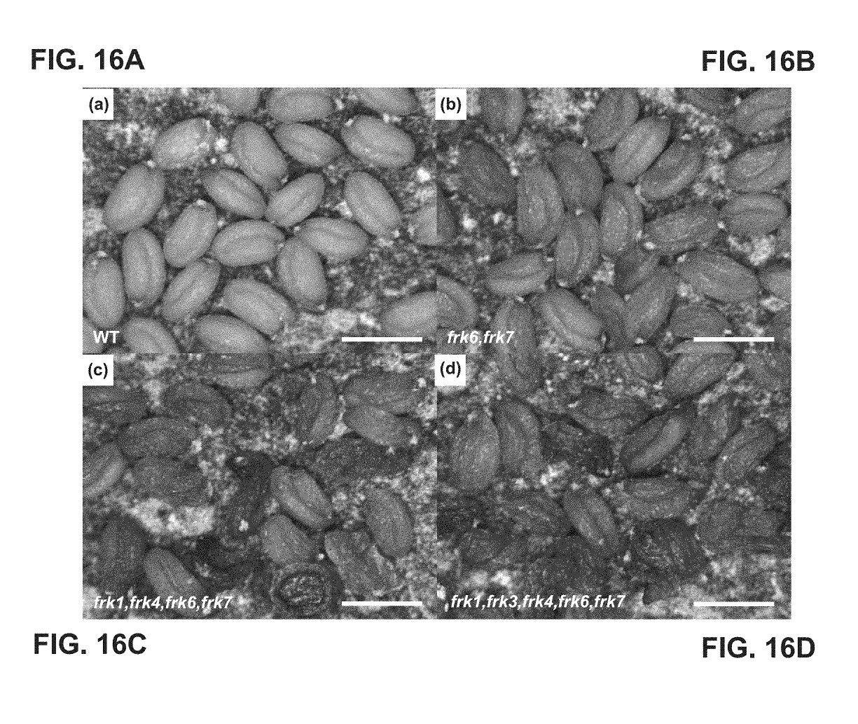

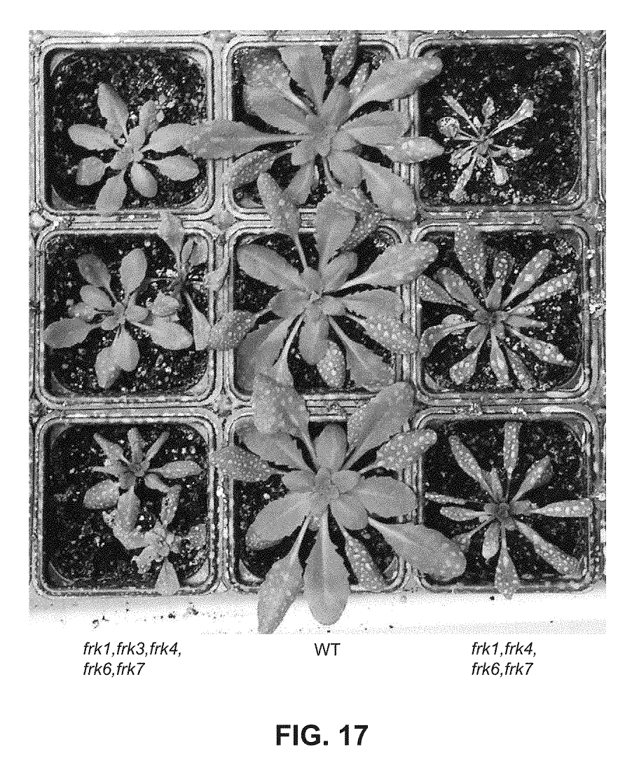

Seeds of the quadruple- and penta-mutant plants that possessed both frk6 and frk7 mutations (frk 1 frk3 frk6 frk7 and frkl frk3 frk4 frk6 frk7, respectively) were wrinkled even more than the frk6 frk7 double-mutant (FIGS. 16A-D) and stopped growing after germinating on ½ MS plates like frk6 frk7 double-mutant. These lines grew normally in soil under long-day conditions (16/8 h, data not shown). However, when grown in soil under even-day (12/12 h) or short-day (8/16 h) conditions, the quadruple- and penta-mutant plants were smaller, with rosette leaves that were half the size of those of the WT plants (FIG. 17). The hypocotyls of the mutants were shorter (2-3 mm) than those of the WT (4-5 mm) plants and, just before bolting, the mutants started wilting, dried out and died (FIG. 17). The wilting phenotype points to lower water conductance, perhaps due to distorted vascular development as was previously shown in tomato FRK mutants (Damari-Weissler et al., 2009; Stein et al., 2016).

### Example 7

### FRK6 FRK7 Seed Phenotype Does Not Stems from Maternal Effect

The phenotype of the frk6 frk7 double-mutant seeds might be a result of altered carbohydrate production or transport from the mother plant to the seeds and/or seed-specific altered carbon metabolism. To determine whether the seed phenotype is due to maternal effect, the homozygote double-mutant frk6 frk7 was crossed with pollen taken from WT, frk6 homozygote or frk7 homozygote plants. The seeds of all of these crosses were normal with regular phenotypes, indicating that the seed phenotype is not caused by reduced sugar production or reduced transport of sugar from the frk6 frk7 homozygote mother plants to the seeds. The seed coats had the maternal genotype, which was frk6 frk7 in all these crosses. However, despite that fact, the seeds were normal, indicating that the wrinkled phenotype is not a result of seed-coat effects.

Then the segregation of the phenotype among the F2 seeds of the cross between the double-mutant and the plastidic frk6 mutant (frk6 frk7 x frk6) was examined. Twenty-five percent of these seeds exhibited the wrinkled double mutant phenotype (Table 5), indicating that this phenotype is caused by a seed-specific effect emanating from the endosperm or from the embryo itself.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of modifying oil content of a plant, the method comprising:
(a) modulating activity or expression of a fructokinase (FRK) in seeds of a plurality of plants; and
(b) selecting a seeds of said plurality of plants or progeny thereof exhibiting said modified oil content as compared to said oil content of seeds of the same species not subjected to step (a).

2. A method of modifying germination rate of a plant, the method comprising:
(a) modulating activity or expression of a fructokinase (FRK) in seeds of a plurality of plants; and
(b) selecting a seeds of said plurality of plants or progeny thereof exhibiting modified germination rate as compared to said oil content of seeds of the same species not subjected to step (a).

3. A method of modifying oil content of an oil seed plant, the method comprising modulating activity or expression of a fructokinase (FRK) in seeds of the oil seed plant.

4. A method of modifying oil content of a plant, the method comprising modulating activity or expression of at least two fructokinases (FRKs) in seeds of the plant, thereby modifying the oil content of the plant.

5. The method of claim 1, wherein said FRK comprises at least two FRKs.

6. The method of claim 1, wherein said modifying and modulating is increasing.

7. The method of claim 1, wherein said modifying and modulating is decreasing.

8. The method of claim 6, wherein said increasing comprises over-expressing a heterologous nucleic acid sequence encoding FRK.

9. The method of claim 8, wherein said nucleic acid sequence encoding said FRK is under a constitutively active promoter.

10. The method of claim 8, wherein said nucleic acid sequence encoding said FRK is under a developmentally regulated promoter active specifically in the embryo.

11. The method of claim 6, wherein said increasing comprises editing an endogenous nucleic acid sequence encoding said FRK.

12. The method of claim 1, wherein said FRK or said at least two FRKs comprise an embryonic expressed FRK or homolog thereof.

13. The method of claim 1, wherein said FRK or said at least two FRKs comprises a cytosolic FRK.

14. The method of claim 1, wherein said FRK or said at least two FRKs comprise comprises a plastid FRK.

15. The method of claim 4, wherein said at least two FRKs comprise AtFRK-6 and AtFRK-7 or a functional homolog thereof.

16. An oil seed plant having been genetically modified or selected to modulate FRK activity in seeds thereof.

17. An oil seed plant obtainable according to the method of claim 1.

18. A seed of the plant of claim 16.

19. Oil of the plant of claim 16.

20. The oil of claim 19, having increased TCA cycle metabolites and/or larger oil bodies as compared to oil of a plant of the same species not subjected to genetic FRK modulation.

21. A method of producing oil, the method comprising:
providing seeds of claim 18; and
extracting oil from said seeds.

22. A nucleic acid construct comprising a nucleic acid agent encoding an FRK under a developmentally regulated promoter specifically active in the embryo.

23. A plant of a plant cell comprising the nucleic acid construct of claim

22.

24. A seed of a plant cell comprising the nucleic acid construct of claim 22.
